(19) United States
(12) Patent Application Publication
(10) Pub. No.: US 2026/0183534 A1
(43) Pub. Date: Jul. 2, 2026

(54) SENSING AND APPLYING STIMULATION IN TIMED RELATIONSHIP

(71) Applicant: INSPIRE MEDICAL SYSTEMS, INC., Golden Valley, MN (US)

(72) Inventors: Heather Orser, Farmington, MN (US); David Dieken, Minneapolis, MN (US); Wim Gewillig, Golden Valley, MN (US); Wondimeneh Tesfayesus, Golden Valley, MN (US); Kevin Verzal, Lino Lakes, MN (US); Kent Lee, Minneapolis, MN (US); John Rondoni, Plymouth, MN (US)

(73) Assignee: INSPIRE MEDICAL SYSTEMS, INC., Golden Valley, MN (US)

(21) Appl. No.: 18/867,857

(22) PCT Filed: May 24, 2023
(86) PCT No.: PCT/US2023/023361

§ 371 Date: Nov. 21, 2024

Related U.S. Application Data
(60) Provisional application No. 63/345,102, filed on May 24, 2022.

(51) Int. Cl. A61N 1/02; A61N 1/36

(52) U.S. Cl. CPC A61N 1/025; A61N 1/3601; A61N 1/3611; A61N 1/36139; A61N 1/36178

(57) ABSTRACT

One example of a device includes a clock, a sensing circuit, and a stimulation circuit. The clock is configured to generate a clock signal. The sensing circuit is configured to periodically sense a signal based on the clock signal. The stimulation circuit is configured to output a stimulation pulse train relative to the periodic sensing of the signal based on the clock signal.

### BACKGROUND

Medical devices, such as implantable medical devices, may include a stimulation engine to provide therapeutic electrical pulses to tissue within a patient. The medical devices may also include sensors to sense a wide variety of phenomenon. For example, implantable medical devices may include sensors to sense physiologic signals, such as signals from the heart, lungs, nerves, etc.

### BRIEF DESCRIPTION OF THE DRAWINGS

FIGS. 1A-1C are block diagrams schematically representing example devices for at least one of sensing and applying stimulation.

FIGS. 2A-2B are block diagrams schematically representing other example devices for sensing and applying stimulation in timed relationship relative to each other.

FIGS. 3A-3C are timing diagrams illustrating example timing relationships between sensing and stimulation.

FIG. 4 is a timing diagram illustrating one example of a timing relationship between sensing and stimulation relative to a clock signal.

FIGS. 5A-5C are flow diagrams illustrating one example of a method for sensing and applying stimulation in a timed relationship relative to each other.

FIGS. 6, 7A, 7B, and 8A each schematically represent different example electrode arrangements for sensing and/or applying stimulation.

FIG. 8B schematically represents an example arrangement for sensing and/or applying stimulation, which includes an implantable medical device, dedicated sensors, non-dedicated sensors, and/or electrodes.

FIG. 9 is a block diagram schematically representing an example care engine.

FIGS. 10A and 10B each are a block diagram schematically representing an example control portion and various example control portion arrangements, respectively.

FIG. 10C is a block diagram schematically representing an example user interface.

FIGS. 11 and 12 are diagrams schematically representing patient anatomy and an example device and/or example method for sensing and/or stimulating an internal superior laryngeal (iSL) nerve, and/or other target tissue.

FIGS. 13, 14, 15, and 16 are diagrams schematically representing patient anatomy and an example device and/or example method for sensing and/or stimulating an infrahyoid muscle (IHM)-innervating nerve, hypoglossal nerve, and/or other target tissue.

FIGS. 17A-17J are diagrams illustrating example sensing protocols and/or stimulation protocols.

FIGS. 18, 19, 20, and 21 are diagrams schematically representing example devices for sensing and/or applying stimulation in context with example patient anatomy.

FIGS. 22A-22E are flow diagrams illustrating example methods for sensing and/or applying stimulation.

FIGS. 23A-23E are diagrams including front and side views schematically representing patient anatomy and example methods relating to collapse patterns associated with upper airway patency.

FIGS. 23F-23I are block diagrams schematically representing example devices and/or example methods relating to collapse patterns associated with upper airway patency.

FIGS. 24 and 25 each are a block diagram schematically representing an example care engine and control portion, respectively.

FIG. 26 is a block diagram schematically representing an example user interface.

### DETAILED DESCRIPTION

In the following detailed description, reference is made to the accompanying drawings which form a part hereof, and in which is shown by way of illustration specific examples in which the disclosure may be practiced. It is to be understood that other examples may be utilized and structural or logical changes may be made without departing from the scope of the present disclosure. The following detailed description, therefore, is not to be taken in a limiting sense. It is to be understood that features of the various examples described herein may be combined, in part or whole, with each other, unless specifically noted otherwise.

At least some examples of the present disclosure are directed to sensing and/or stimulation. In some examples, the sensing and stimulation are coordinated relative to each other, even when timing of delivery of the stimulation is not based on information received from the sensing. In some examples, the sensing and stimulation may be performed relative to a common target tissue such as the same nerve, same muscle, combination thereof, and/or other types of body tissues in proximity to such nerves, muscles, etc. In some examples, the sensing and stimulation may be performed on different target tissues, e.g., not the same target tissue.

At least some examples of the present disclosure are directed to devices (e.g., implantable medical devices) including a clock to generate a clock signal, a sensing circuit, and a stimulation circuit. The sensing circuit is configured to periodically sense a signal (e.g., such as a signal from the heart, lungs, nerves, etc. of a patient) based on the clock signal. The stimulation circuit is configured to output a stimulation pulse train (e.g., a plurality of stimulation pulses) based on the clock signal such that the stimulation pulse train is output in a timed relationship relative to the sensing. By sensing and applying stimulation in a timed relationship, the sensing and stimulation remain synchronous over time.

In some examples, the occurrence of a sensing signal is coordinated relative to the occurrence of a stimulation signal to minimize any potential stimulation artifacts present in the sensed signal and may increase consistency of the magnitude and impact of the stimulation artifacts on the sensing signal. In some examples, a (master) clock may be used to ensure stimulation timing remains consistent relative to the sampling time of the sensing circuit.

In some examples, the occurrence of a sensing signal is independent of the occurrence of a stimulation signal. For example, sensing may be timed independent of the stimulation. In some such examples, the sensing may be performed using techniques in which the stimulation artifacts are not or minimally are present in the sensed signal, such that the stimulation artifacts do not impact the sensing signal.

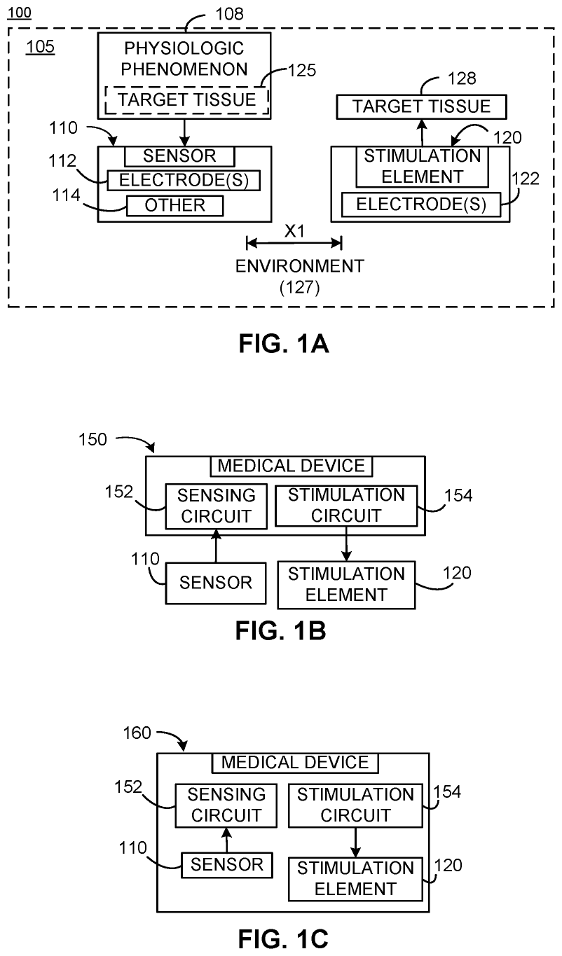

These examples, and additional examples, are described below in association with FIGS. 1A-26.

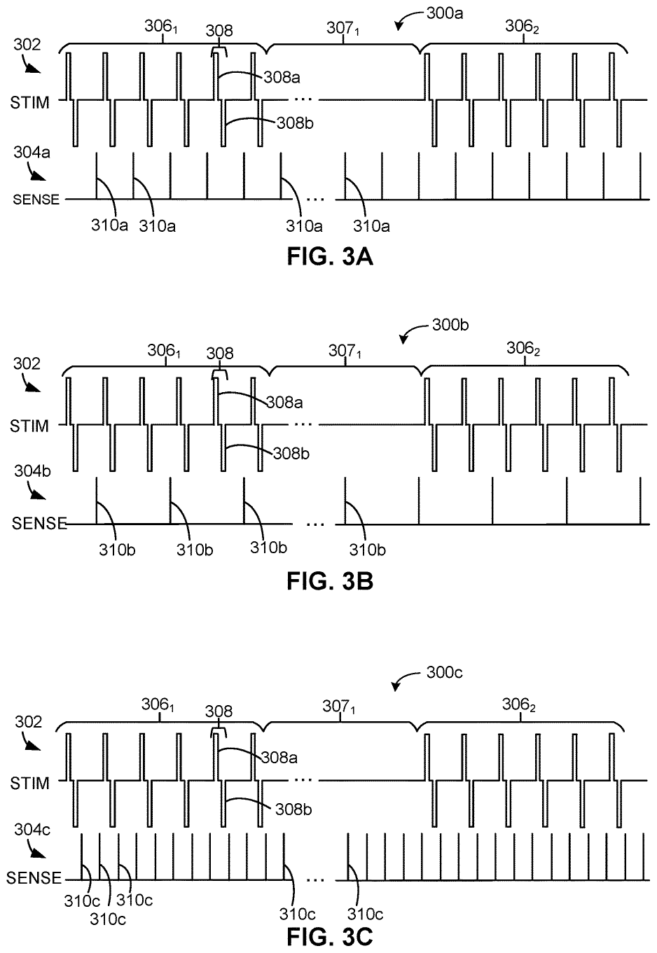

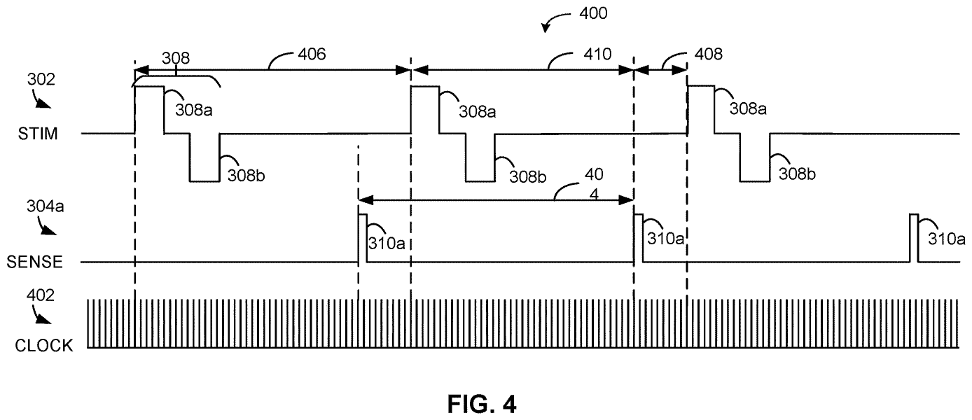

As used herein a “stimulation pulse train” includes a plurality (e.g., two or more) of stimulation pulses, where each stimulation pulse may include a cathodic portion and an anodic portion as described below at least with reference to FIGS. 3A-4.

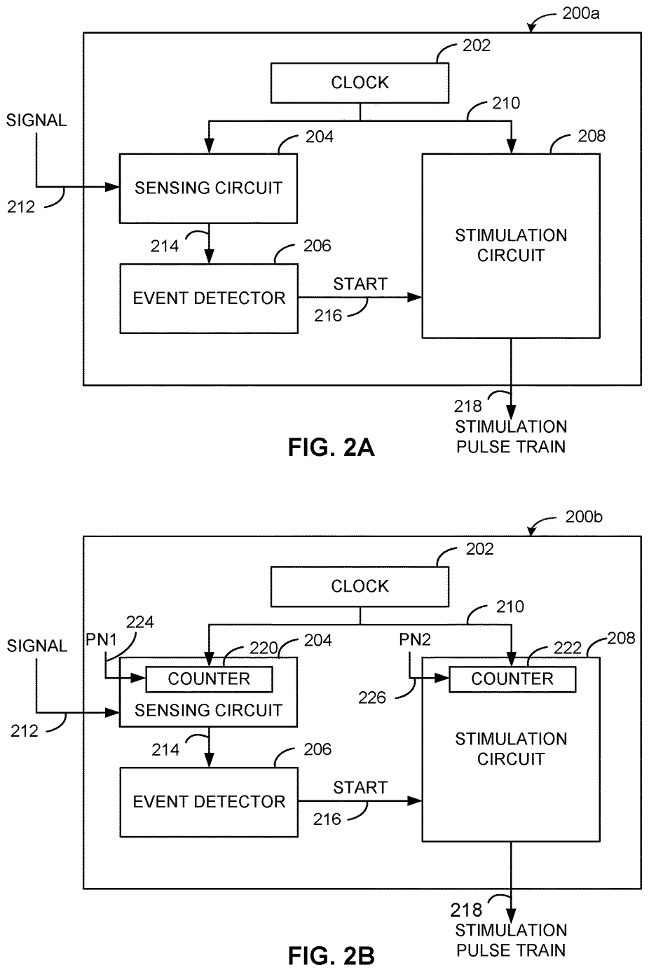

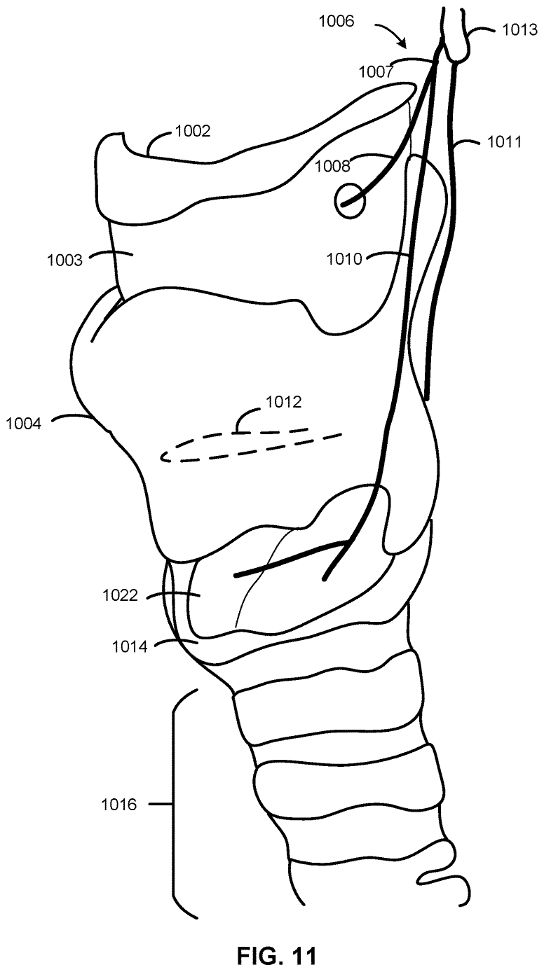

FIG. 1A is a diagram 100 schematically representing an example device (and/or example method) 105 for sensing and applying stimulation, which may be in timed relationship relative to each other. Various aspects of such timing by which sensing and stimulation may be coordinated are further described below in association with at least FIGS. 2A-10C. In addition, at least some of these coordinated timing examples are applicable to various examples of sensing and stimulation, are further described below in association with at least FIGS. 11-26.

As shown in FIG. 1A, in some examples the example device 105 may comprise a sensor 110 and a stimulation element 120 located within an environment 127. In general terms, in some examples, the sensor 110 and stimulation element 120 are located within a proximity relative to each other such that applying stimulation during sensing (or in close temporal relation to such sensing) or vice versa may affect the performance, quality, etc. of such respective stimulation and/or sensing such that some examples of the present disclosure may direct coordinated timing of such stimulation and sensing to ameliorate such effects on performance, quality, etc.

In some examples, the application of stimulation and the sensing are spaced apart from each other within the environment 127 by a distance, as represented by distance arrow X1, within which the application of stimulation and performance of sensing may benefit from coordinated timing. It will be understood that in some examples, the distance X1 may be zero or negligible such that the stimulation and sensing are in sufficiently close proximity to be considered co-located.

In some examples, the environment 127 may comprise a head-and-neck region, a pectoral region, an abdominal region, any other body region, and/or combinations thereof. In some examples, a target tissue 128 may be located within, and/or physiologic phenomenon 108 may occur within, at least some of these example regions. In some such examples, within this example environment 127, the example method 105 may comprise treating sleep disordered breathing such as, but not limited to, obstructive sleep apnea, central sleep apnea, multi-type apneas, etc.

In some examples, the environment 127 may comprise a pelvic region. In some examples, the target tissue 128 may be located within, and/or the physiologic phenomenon 108 may occur within, at least the pelvic region. In some such examples, the example method 105 may comprise treating pelvic dysfunctions such as, but not limited to, various forms of incontinence (urinary urgency, urinary stress, fecal, and the like) occurring within this example environment 127.

It will be understood that, in some examples, the environment 127 may comprise any portion of the patient anatomy in which the application of stimulation and performance of sensing may be enhanced via coordinated timing of such stimulation and sensing.

The sensor 110 may sense (e.g., detect) physiologic phenomenon 108 associated with the environment 127 while the stimulation element 120 may deliver (e.g., apply) stimulation to a target tissue 128 of, or within, the environment 127. In some examples, the target tissue 128 may comprise a nerve portion(s), a muscle portion(s), a combination of nerve portion(s) and muscle portion(s), a neuromuscular junction of nerve portion(s) and muscle portion(s), and/or combinations thereof.

In some examples, both the sensor 110 and the stimulation element 120 are implanted within a patient's body, which forms part of the environment 127.

However, in some examples, one or both of the sensor 110 and the stimulation element 120 may be external to the patient's body, such that the environment 127 comprises at least both internal portions and external portions of the patient's body. In some such examples, the environment 127 also may comprise an area which does not comprise the patient's body but which is in close proximity to the patient's body.

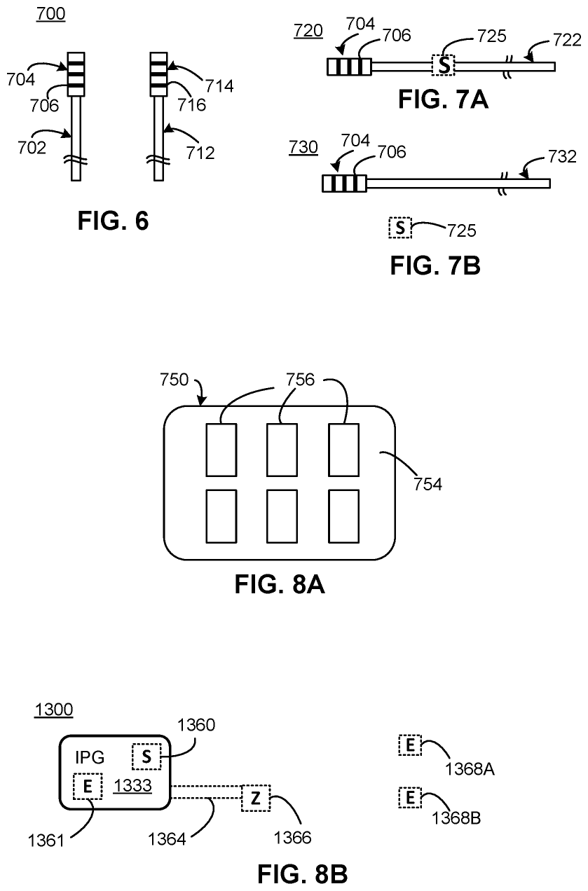

In some examples, the sensor 110 may comprise an electrode(s) 112 and/or other elements 114 for sensing, as further described later in association with at least FIGS. 6-9.

In some examples, the stimulation element 120 may comprise an electrode(s) 122 for delivering a stimulation signal to the target tissue 128.

In some examples, the electrode(s) 122 used for applying stimulation also may be used for sensing, and as such also may comprise electrode(s) 112, as further described later. Similarly, the electrode(s) 112 used for sensing also may be used for applying stimulation, and as such also may comprise electrode(s) 122. However, in some examples, the sensing electrode(s) 112 are used solely for sensing and the stimulation electrode(s) 122 are used solely for applying stimulation. Various example implementations incorporating these permutations and/or other permutations are described later in association with at least FIGS. 6-9.

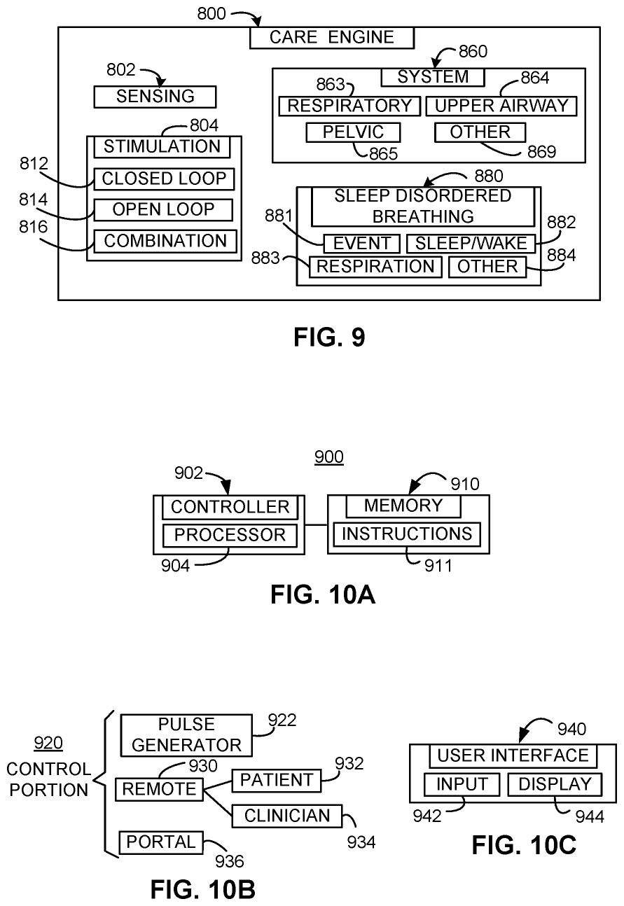

In some examples, other elements 114 used for sensing may comprise a sensing element which does not depend on electrode(s) 112 for sensing. For example, as further described later in association with at least FIGS. 6-10, such other sensing elements 114 may comprise a pressure sensor (e.g., differential pressure), an accelerometer, and/or other sensing elements, such as further described in association with at least FIG. 9.

In some examples, the electrode(s) 112 and/or other sensing elements 114 (e.g., accelerometer) may be used to sense one or more of motion, activity, body position (e.g., posture), respiration, heart rate, etc., at least some of which may be used to detect disordered breathing and/or other disease burdens. At least some further examples of other sensing elements 114 and/or physiologic phenomenon sensed via such elements 114 (and/or electrode(s) 112) are described later in association with at least FIG. 9.

In some example implementations, the sensor 110 may comprise both electrode(s) 112 and other sensing element(s) 114, which may be operated independently from each other or in combination with each other.

In some examples, the stimulation electrode(s) 122 may take a wide variety of forms, and may be incorporated within a wide variety of different types of stimulation elements, at least some of which are described in association with at least FIGS. 6-9.

FIG. 1B is a block diagram schematically representing a medical device 150, which comprises one example implementation of an example device (and/or example method) for sensing and applying stimulation in timed relationship relative to each other. In some examples, the medical device 150 may comprise at least some of substantially the same features and attributes as the example device 105 in FIG. 1A.

As shown in FIG. 1B, the medical device 150 may comprise a sensing circuit 152 to receive sensed physiologic information from sensor 110 and a stimulation circuit 154 to deliver a stimulation signal to the stimulation element 120 for application to a target tissue 128 (FIG. 1A).

In some examples, the sensed physiologic information received at the sensing circuit 152 from the sensor 110 may be used to determine when to start and/or terminate stimulation, a duration of such stimulation, and/or other parameters, such as stimulation amplitude and/or selection of the target tissue 128. However, in some examples, this received, sensed physiologic information may be used for monitoring physiologic functions, disease burden, etc. without necessarily being used to determine stimulation functions (e.g., start, terminate, duration, etc.), as further described below.

In some examples, the sensed physiologic information may comprise information relating to respiration, sleep, posture, and/or disease burden (e.g., severity of disordered breathing), such as when the environment 127 includes body regions relating to breathing. In some examples in which at least respiration comprises the sensed physiologic information, the sensed respiration may comprise respiration parameters, such as respiratory waveform morphology, inspiratory phase, expiratory phase (including active expiration and expiratory pause), and/or other respiratory information, as further described later. In some examples, the sensed respiratory information may be used to determine the start time, end time, and/or duration of stimulation relative to a respiratory cycle generally and/or specifically in relation to fiducials of the respiratory waveform. In some examples, such fiducials may comprise a start time, end time, duration, crossing points, peaks, and/or other parameters of each of an inspiratory phase and an expiratory phase. In some examples, this sensed respiratory information may be used to synchronize the stimulation with a particular portion of the respiratory cycle such as, but not limited to, the inspiratory phase, the expiratory phase, and portions of the inspiratory phase and/or the expiratory phase. In some examples, the sensed respiratory information may be used to determine timing and/or duration of the stimulation, amplitude of the stimulation, and/or selection of the target tissue 128 to be stimulated, as further described herein. In some examples, these example arrangements may sometimes be referred to as closed-loop stimulation, as further described later.

In some examples in which the sensed physiologic information relates to breathing, the target tissue 128 (FIG. 1A) to be stimulated may comprise target tissues relating to breathing, and in particular sleep disordered breathing. In some such examples, the target tissues to be stimulated may comprise upper airway patency-related motor nerves and muscles, which may comprise a hypoglossal nerve, infrahyoid muscle (IHM)-innervating nerve, and/or other nerves and the muscles innervated by the aforementioned example nerves, associated neuromuscular junctions, etc. In some examples, upper airway patency-related motor nerves may include nerves that stimulate muscles associated with increasing, restoring, or maintaining upper airway patency to promote respiration. In some examples, the target tissues 128 may comprise nerves, muscles, etc. not directly related to upper airway patency, such as the phrenic nerve, diaphragm, or other nerves/muscles relating to respiration. In such examples, the target tissues may comprise the phrenic nerve and/or the diaphragm muscles.

In some examples, the target tissues may comprise nerves, which when stimulated, elicit (via the central nervous system (CNS)) a reflex opening response which activates at least some of the above-identified nerves and/or muscles to facilitate respiration to prevent and/or overcome sleep disordered breathing, which are sometimes herein referred to as “upper airway reflex-related sensory nerves”. In some examples, upper airway reflex-related sensory nerves may include nerves associated with carrying sensory information that elicits a reflex opening response. In some examples, the targeted afferent nerve fiber(s) may be selectively stimulated by selecting a stimulation location associated with afferent nerve fibers, such as an afferent branch and/or steering to stimulate selected afferent nerve fibers within a nerve branch. Example upper airway reflex-related sensory nerves include the internal superior laryngeal (iSL) nerve and the glossopharyngeal nerve. As previously noted, the target tissues 128 may comprise nerve portion(s), muscle portion(s), a combination of nerve portion(s) and muscle portion(s), neuromuscular junction(s) of nerve portion(s) and muscle portion(s), and/or combinations thereof. In some examples, the stimulation signal may comprise sufficient strength (and/or other characteristics) to cause suprathreshold contraction of the target muscle portion such as, but not limited to, stimulation of the hypoglossal nerve resulting in protrusion of the tongue (e.g., genioglossus muscle), stimulation of the IHM-innervating nerve resulting in contraction of other upper airway muscles. In some such examples, such stimulation may maintain and/or increase upper airway patency to treat at least obstructive sleep apnea.

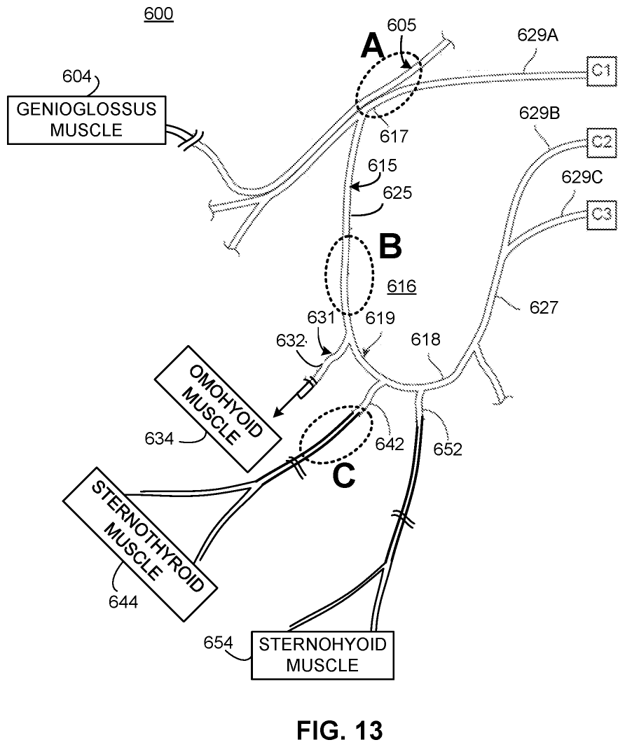

Further details regarding at least some of these anatomical structures and relationships such as (but not limited to) the IHM-innervating nerve, hypoglossal nerve, etc. are described later in association with at least FIG. 13, as well as in association with at least FIGS. 11-16, 17A-17J, and 18-21.

In some examples, the sensed physiologic information may comprise information relating to bladder pressure/volume, urgency, posture, body position, voiding, and/or disease burden (e.g., severity of urinary incontinence and/or fecal incontinence), etc., such as when the environment 127 includes body regions relating to pelvic dysfunction. In some examples in which at least bladder volume and/or bladder pressure comprises the sensed physiologic information, the sensed information may comprise bladder function-related waveform morphology, infilling period, voiding event, and/or other bladder function-related information, as further described later. In some examples, the sensed bladder function-related information may be used to determine the start time, end time, and/or duration of stimulation relative to the sensed bladder function-related information. In some examples, this sensed bladder function-related information may be used to synchronize the stimulation with particular portions of bladder functions and/or intended bladder functions. In some examples, these example arrangements may sometimes be referred to as closed-loop stimulation, as further described later.

In some examples in which the sensed physiologic information relates to pelvic dysfunction, the target tissue 128 (FIG. 1A) to be stimulated may comprise target tissues relating to urination, defecation, etc., and in particular urinary incontinence and/or fecal incontinence such as, but not limited to, stress incontinence. In some such examples, these tissues may comprise nerves and muscles associated with voiding and/or prevention of voiding, with such nerves and/or muscles being associated with at least the external urinary sphincter and/or external anal sphincter. Among other examples, at least the pudendal nerve comprises one target tissue innervating such muscles, with the target including the pudendal nerve trunk, the deep perineal branch, and/or other portions of the pudendal nerve. At least some further examples of target tissues may comprise the hypogastric nerve and/or pelvic splanchnic nerve. In some examples, the target tissues 128 may comprise nerves, muscles, etc. not directly related to incontinence, such as other nerves/muscles relating to pelvic dysfunction. As previously noted, the target tissues 128 may comprise nerve portion(s), muscle portion(s), a combination of nerve portion(s) and muscle portion(s), neuromuscular junction(s) of nerve portion(s) and muscle portion(s), and/or combinations thereof. In some examples, the stimulation signal may comprise sufficient strength (and/or other characteristics) to cause suprathreshold contraction of the target muscle portion such as, but not limited to, stimulation of at least a portion of the pelvic function-related nerve resulting in contraction of a respective one of the sphincter muscles and/or relaxation of a respective one of the sphincter muscles, or stimulation of the pertinent nerve resulting in contraction (or relaxation) of other pelvic muscles. In some such examples, such stimulation may be delivered to treat at least urinary incontinence and/or fecal incontinence such as, but not limited to, stress incontinence.

In some examples, an event may be detected or determined from the sensed physiologic information with the event being used to coordinate timing of the stimulation signal and the sensing signal. In some such examples, the event may comprise the same physiologic information on which the closed-loop stimulation is based.

In some examples, at least some of the aforementioned principles regarding sensing and/or stimulation from these example implementations may be applied to other body regions, organs, functions, etc.

In some examples, a timing of sensing and stimulation may be coordinated without performing closed-loop stimulation, i.e., may be coordinated while performing open-loop stimulation. In some such examples, even though each (or at least some) stimulation periods are not triggered or initiated based on sensed information (e.g., respiratory for breathing, pressure/volume for pelvic, etc.), the sensing may still be performed to determine disease burden and/or other physiologic information desirable to monitor. In some examples, these example arrangements may sometimes be referred to as open-loop stimulation, as further described later. In these example arrangements, an event may be detected or determined from the sensed physiologic information with the event being used to coordinate timing of the stimulation signal and the sensing signal, except with the event (e.g., sensed physiologic information) not being used to trigger or initiate stimulation but instead for timing the sensing and stimulation relative to each other to enhance performance, quality, etc. of the sensing and/or stimulation.

In some examples of open loop stimulation, an event may be detected or determined from the sensed physiologic information with the event being used to coordinate timing of the stimulation signal and the sensing signal. However, in some such examples, the event is not used to perform closed-loop stimulation such as timing stimulation to coincide with certain phases (e.g., inspiration, expiration), or portions of such phases, transitions between such phases, of sensed respiration, etc.

With further reference to FIG. 1B, the sensing circuit 152 and/or the stimulation circuit 154 in environment 127 may be external to the patient's body or implanted within the patient's body. In some such examples, the sensor 110 and the stimulation element 120 may be implanted within the patient's body while one or both of the sensing circuit and the stimulation circuit are external to the patient's body, with wired and/or wireless communication occurring between the implanted elements and externally-located elements to transfer power and/or data. In some examples, both circuits may comprise part of the same medical device such as, but not limited to, a pulse generator.

With further reference to FIG. 1B, in some examples the medical device 150 may comprise a pulse generator, at least some portions of which may be implantable. In some such examples in which at least the stimulation circuit 154 is implanted within the patient's body, the medical device 150 may sometimes be referred to as an implantable pulse generator (IPG).

FIG. 1C is a block diagram schematically representing a medical device 160, which comprises one example implementation of an example device (and/or example method) for sensing and applying stimulation in timed relationship relative to each other. In some examples, the medical device 160 may comprise at least some of substantially the same features and attributes as the example medical device 150 of FIG. 1B, except comprising the sensor 110 and/or the stimulation element 120 being incorporated into the medical device 160 instead of being external (e.g., separate from) to the medical device, as in the example of FIG. 1B. In some examples, the sensor 110 and/or the stimulation element 120 may be contained within a housing of the medical device 160, while in some examples, the sensor 110 and/or the stimulation element 120 may be external to the housing, such as being located on an exterior surface of the housing of the medical device 160. In some such examples, the sensor 110 and/or the stimulation element 120 may sometimes be referred to as being on-board the medical device 160.

In some examples in which the medical device 160 comprises an implantable pulse generator which includes sensing circuit 152, sensor 110, stimulation circuit 154, and stimulation element 120, the medical device is sized and/or shaped for chronic implantation in locations (e.g., head-and-neck, intravascular) which are substantially smaller than traditional implant locations for an IPG like a subcutaneous pocket in a pectoral or abdominal location. In some such examples, the sensor 110 and the stimulation element 120 may be considered to be co-located within environment 127 (FIG. 1A). In some of these example arrangements, the medical device 160 may comprise or be referred to as a microstimulator.

Similarly, it will be further understood that in some examples, the medical device 150 of FIG. 1B (incorporating the sensing circuit 152 and the stimulation circuit 154) may be sized and/or shaped for chronic implantation in locations (e.g., head-and-neck, intravascular, etc.) which are substantially smaller than traditional implant locations for an IPG like a subcutaneous pocket in a pectoral or abdominal location. In some such examples, the medical device 150 may comprise or be referred to as a microstimulator.

In some examples, the medical devices 150, 160 may comprise a power element, which may comprise a non-rechargeable power source (e.g., battery), a re-chargeable power source, a power storage element to receive power wirelessly from an external source, and/or energy harvesting/storage elements.

With further reference to FIGS. 1A-1C, the stimulation applied from the stimulation circuit 154 via stimulation element 120 may be controlled according to an amplitude, frequency, pulse width, duty cycle, duration, and the like to achieve desired therapeutic efficacy, which may depend on a region of the body, a type, size/shape, location of target tissue, number/location/size of stimulation elements, etc. In some examples, a combination of the stimulation circuit 154 and the stimulation element 120 may sometimes be referred to as a stimulation portion.

In some examples, at least the sensing circuit 152 and/or stimulation circuit 154 may comprise at least some of substantially the same features and attributes as, comprise an example implementation of, or be complementary to the later described example control portion 900 (FIGS. 10A), 920 (FIG. 10B).

FIG. 2A is a block diagram schematically representing an example device 200a (e.g., IPG). In some examples, the device of FIG. 2A may comprise at least some of substantially the same features and attributes as, or an example implementation of, the example arrangements previously described in association with at least FIGS. 1A-1C. As shown in FIG. 2A, the device 200a includes a clock 202, a sensing circuit 204, an event detector 206, and a stimulation circuit 208. The clock 202, such as a master clock, is electrically coupled to the sensing circuit 204 and the stimulation circuit 208 through a signal path 210. An input of the sensing circuit 204 is electrically coupled to a signal path 212 (e.g., coupled to sensor 110 of FIGS. 1A-1C) to receive a signal. An output of the sensing circuit 204 is electrically coupled to an input of the event detector 206 through a signal path 214. An output of the event detector 206 is electrically coupled to an input of the stimulation circuit 208 through a signal path 216. An output of the stimulation circuit 208 is electrically coupled to a signal path 218 (e.g., coupled to stimulation element 120 of FIGS. 1A-1C) to apply a stimulation pulse train.

The clock 202 generates a clock signal. In some examples, the clock 202 may generate a clock signal having a frequency within a range between about 25 kHz and about 40 kHz. The clock 202 may include a crystal oscillator and associated circuitry to generate a clock signal having a predetermined frequency. One example of a clock signal is described later at least with reference to FIG. 4.

The sensing circuit 204 periodically senses (e.g., samples) a signal on signal path 212 based on the clock signal. In some examples, as described in additional detail below with reference to at least FIGS. 3A-4, the sensing circuit 204 senses the signal on signal path 212 beginning every first predetermined number of cycles of the clock signal. For example, the sensing circuit 204 may sense the signal on signal path 212 on an even number of clock cycles between 32 clock cycles and 30,000 clock cycles of the clock signal. The duration of the sensing of the signal on signal path 212 may exceed one cycle of the clock signal, such as 2, 5, 10, 20, or more cycles of the clock signal. In some examples, the sensing circuit 204 senses a physiologic signal due to a physiologic phenomenon 108 (FIG. 1A). The physiologic signal may include a cardiac signal, a muscle signal, or a nerve signal.

As further described later in association with at least FIGS. 11-26, in some examples, the sensing circuit 204 may sense the physiologic signal without use of a clock signal and/or using a clock signal which is timed independent of stimulation. In such examples, the sensing circuit 204 may sense the physiologic signal independent of timing of stimulation or stimulation may be delivered independent of sensing.

With further reference to FIG. 2A, the event detector 206 may generate a start signal on signal path 216 in response to detecting an event. In some examples, the event detector 206 may detect an event based on an output from the sensing circuit 204 on signal path 214 relating to the sensed signal. The event may be a physiologic event of a patient, such as inspiration or expiration of the patient, or other event as previously described. In some examples, the event detector 206 may be used to enable closed-loop stimulation, where stimulation is applied relative to (e.g., triggered by, based on, timed with, in response to, synchronized with, etc.) detected specific physiologic events (e.g., inspiration) as previously described. In other examples, the event detector 206 may be used to enable open-loop stimulation, where stimulation is not applied in response to specific physiologic events, but rather based on other predetermined timing parameters and/or other parameters without synchronizing the stimulation with a sensed physiologic phenomenon (e.g., an inspiratory phase of a respiration cycle). In some examples, the event detector 206 may generate a signal, in response to detecting the event, that controls the stimulation (via the stimulation circuit 208), such as setting the timing of stimulation, the duration of stimulation, the stimulation amplitude, and/or selection of target tissue to apply the stimulation to.

In some examples, other detectable events which may be used to generate a start signal (on signal path 216) may comprise events such as, but not limited to, an external telemetry signal, a signal trigger from an accelerometer based on movement or physical disturbances, a measured impedance discontinuity, or a sensed physiologic signal. Accordingly, the events may be physiologic events and/or non-physiologic events.

The stimulation circuit 208 outputs a stimulation pulse train on signal path 218 relative to the periodic sensing of the signal on signal path 212 by sensing circuit 204 based on the clock signal. As described in additional detail below with reference to at least FIGS. 3A-4, the stimulation pulse train includes a plurality of stimulation pulses. In some examples, the stimulation circuit 208 outputs each stimulation pulse beginning every second predetermined number of cycles of the clock signal. For example, the stimulation circuit 208 may output a stimulation pulse of a stimulation pulse train on signal path 218 on an even number of clock cycles between 32 clock cycles and 30,000 clock cycles of the clock signal. Accordingly, an interval between the periodic sensing of the signal by sensing circuit 204 and a stimulation pulse of the stimulation pulse train output by the stimulation circuit 208 is constant. In some examples, the stimulation circuit 208 begins a first stimulation pulse of the stimulation pulse train a third predetermined number of cycles of the clock signal after the beginning of a previous sensing of the signal in response to the start signal on signal path 216. In this way, no matter when the start signal is received, the stimulation circuit 208 waits to output the first stimulation pulse of the stimulation pulse train such that the interval between the periodic sensing of the signal by the sensing circuit 204 and each stimulation pulse remains constant. Therefore, the time (and the number of clock cycles) between receiving the start signal and the start of the first stimulation pulse of the stimulation pulse train may vary by up to the first predetermined number of clock cycles (e.g., the clock cycles between sensing operations).

In one example, the stimulation circuit 208 outputs the stimulation pulse train to a nerve of a patient, such as a nerve that innervates the tongue and soft palate of the patient. In other examples, the stimulation circuit 208 may output the stimulation pulse train to other target tissue 128 (FIG. 1A) as previously described.

In one example, as described in additional detail below with reference to at least FIG. 3A, the first predetermined number of clock cycles between sensing (e.g., sampling) operations equals the second predetermined number of clock cycles between stimulation pulses of the stimulation pulse train. Thus in this example, the sensing operations alternate with each stimulation pulse of the pulse train in a one-to-one (1:1) alternating relationship. In another example, as described in additional detail below with reference to at least FIG. 3B, the first predetermined number of clock cycles between sensing operations is an integer multiple of the second predetermined number of clock cycles between stimulation pulses of the stimulation pulse train. Thus in this example, each sensing operation alternates with multiple (e.g., two or more) stimulation pulses of the stimulation pulse train in an alternating (e.g., 1:2, 1:3, 1:4, etc.) relationship. In yet another example, as described in additional detail below with reference to at least FIG. 3C, the first predetermined number of clock cycles between sensing operations is an integer divisor of the second predetermined number of clock cycles between the stimulation pulses of the stimulation pulse train. Thus in this example, multiple (e.g., two or more) sensing operations alternate with each stimulation pulse of the stimulation pulse train in an alternating (e.g., 2:1, 3:1, 4:1, etc.) relationship.

FIG. 2B is a block diagram schematically representing another example of a device 200b for sensing and applying stimulation in timed relationship relative to each other. Device 200b is similar to device 200a previously described and illustrated with reference to FIG. 2A, except that device 200b also includes a first counter 220 and a second counter 222. The sensing circuit 204 includes the first counter 220, and the stimulation circuit 208 includes the second counter 222.

A first input of the first counter 220 is electrically coupled to the clock 202 through the signal path 210 to receive the clock signal, and a second input of the first counter 220 is electrically coupled to a signal path 224 to receive the first predetermined number (PN1). The first counter 220 counts cycles of the clock signal. In response to the count of the first counter 220 equaling the first predetermined number of cycles, the sensing circuit 204 begins to sense (e.g., sample) the signal on signal path 212 and resets the first counter 220. Thus, sensing circuit 204 senses the signal on signal path 212 every first predetermined number of cycles of the clock signal.

A first input of the second counter 222 is electrically coupled to the clock 202 through the signal path 210 to receive the clock signal, and a second input of the second counter 222 is electrically coupled to a signal path 226 to receive the second predetermined number (PN2). The second counter 222 counts cycles of the clock signal. In response to the count of the second counter 222 equaling the second predetermined number of cycles and a start signal on start signal path 216, the stimulation circuit 208 begins a first stimulation pulse of the stimulation pulse train on signal path 218 and resets the second counter 222. In response to the count of the second counter 222 equaling the second predetermined number of cycles and the stimulation pulse train being in progress, the stimulation circuit 208 begins the next stimulation pulse of the stimulation pulse train and resets the second counter 222. In response to the count of the second counter 222 equaling the second predetermined number of cycles, no start signal on start signal path 216, and no stimulation pulse train currently in progress, the stimulation circuit 208 resets the second counter 222. Thus, stimulation circuit 208 outputs a stimulation pulse on signal path 218 every second predetermined number of cycles of the clock signal while a stimulation pulse train is in progress.

The count of the first counter 220 may be offset with respect to the count of the second counter 222 by the third predetermined number of cycles. Thus, each stimulation pulse follows the previous sensing operation by the third predetermined number of cycles. The sensing circuit 204 may continue to sense the signal on signal path 212 between stimulation pulse trains every first predetermined number of cycles of the clock signal, such that any number of sensing operations may be performed between stimulation pulse trains. The event detector 206 may detect an event and generate the start signal at any time, either while a stimulation pulse train is in progress and/or after a stimulation pulse train is complete. In any case, stimulation circuit 208 and sensing circuit 204 maintain the timing relationship between sensing operations and stimulation pulses of a stimulation pulse train.

FIG. 3A is a timing diagram 300a illustrating one example of a timing relationship between sensing and stimulation. A stimulation signal (STIM) 302, which may be applied by stimulation element 120 of FIGS. 1A-1C or on signal path 218 of FIGS. 2A-2B, includes a plurality of stimulation pulse trains 306 separated by non-stimulation phases 307. Two stimulation pulse trains 306₁ and 306₂ and one non-stimulation phase 307₁ are shown in FIG. 3A. Each stimulation pulse train 306₁ and 306₂ includes a plurality of stimulation pulses 308, where each stimulation pulse 308 includes a cathodic portion 308a and an anodic portion 308b. While the cathodic portion 308a and the anodic portion 308b of each stimulation pulse 308 are rectangular in shape in the example shown in FIG. 3A, in other examples, the stimulation pulses 308 within each stimulation pulse train 306 may have other suitable shapes. While each stimulation pulse train 306₁ and 306₂ shown in FIG. 3A includes six stimulation pulses 308, in other examples, each stimulation pulse train 306₁ and 306₂ may include another suitable number (e.g., 2, 3, 4, 5, 7, 8, 9, 10, etc.) of stimulation pulses 308.

A signal (e.g., physiologic signal) is sensed (e.g., sampled) by sensing circuit 152 of FIGS. 1B-1C or by sensing circuit 204 of FIGS. 2A-2B periodically as indicated by sense sampling time (SENSE) 304a. The sense sampling time 304a includes periodic sense operations 310a. In this example, each stimulation pulse 308 alternates with a sense operation 310a in a one-to-one (1:1) relationship. Between stimulation pulse trains 306₁ and 306₂, the sensing operations 310a continue at the same rate during the non-stimulation phase 307₁. In the example of FIG. 3A, the number of clock cycles between stimulation pulses 308 equals the number of clock cycles between sensing operations 310a. Additional features of stimulation signal 302 and sense sampling time 304a will be described below with reference to FIG. 4.

FIG. 3B is a timing diagram 300b illustrating another example of a timing relationship between sensing and stimulation. The stimulation signal 302 of timing diagram 300b was previously described and illustrated with reference to FIG. 3A. In this example, a signal (e.g., physiologic signal) is sensed (e.g., sampled) by sensing circuit 152 of FIGS. 1B-1C or by sensing circuit 204 of FIGS. 2A-2B periodically as indicated by sense sampling time (SENSE) 304b. The sense sampling time 304b includes periodic sense operations 310b. In this example, multiple stimulation pulses 308 alternate with a sense operation 310b in a two-to-one (2:1) relationship. Between stimulation pulse trains 306₁ and 306₂, the sensing operations 310b continue at the same rate during the non-stimulation phase 307₁. In the example of FIG. 3B, the number of clock cycles between stimulation pulses 308 equals one half the number of clock cycles between sensing operations 310b.

FIG. 3C is a timing diagram 300c illustrating another example of a timing relationship between sensing and stimulation. The stimulation signal 302 of timing diagram 300c was previously described and illustrated with reference to FIG. 3A. In this example, a signal (e.g., physiologic signal) is sensed (e.g., sampled) by sensing circuit 152 of FIGS. 1B-1C or by sensing circuit 204 of FIGS. 2A-2B periodically as indicated by sense sampling time (SENSE) 304c. The sense sampling time 304c includes periodic sense operations 310c. In this example, each stimulation pulse 308 alternates with multiple sense operations 310c in a one-to-two (1:2) relationship. Between stimulation pulse trains 3061 and 306₂, the sensing operations 310c continue at the same rate during the non-stimulation phase 307₁. In the example of FIG. 3C, the number of clock cycles between stimulation pulses 308 equals two times the number of clock cycles between sensing operations 310c.

FIG. 4 is a timing diagram 400 illustrating one example of a timing relationship between sensing and stimulation relative to a clock signal. Timing diagram 400 includes additional details of timing diagram 300a of FIG. 3A. While FIG. 4 relates to FIG. 3A, similar features are also applicable to timing diagram 300b of FIG. 3B and timing diagram 300c of FIG. 3C. The clock signal (CLOCK) 402 may be provided by clock 202 of FIGS. 2A-2B. A sensing operation 310a begins every first predetermined number of clock cycles of the clock signal 402 as indicated at 404. A stimulation pulse 308 begins every second predetermined number of clock cycles of the clock signal 402 as indicated at 406. A first stimulation pulse 308 and each subsequent stimulation pulse 308 within each stimulation pulse train begins a third predetermined number of cycles of the clock signal 402 after the beginning of a previous sensing operation as indicated at 408. Each sensing operation 310a begins a fourth predetermined number of cycles of the clock signal 402 after the beginning of a previous stimulation pulse 308 of the stimulation pulse train as indicated at 410.

In the example shown in FIG. 4 (and FIG. 3A), the first predetermined number of clock cycles 404 equals the second predetermined number of clock cycles 406. In the example shown in FIG. 3B, the first predetermined number of clock cycles equals two times the second predetermined number of clock cycles. In the example shown if FIG. 3C, the first predetermined number of clock cycles equals one half the second predetermined number of clock cycles. Also in the example shown in FIG. 4, the third predetermined number of clock cycles 408 is less than the fourth predetermined number of clock cycles 410, such that each sensing operation 310a is closer to the beginning of the next stimulation pulse 308 than to the end of the previous stimulation pulse 308. In this way, stimulation artifacts due to the stimulation pulses 308 may be minimized prior to each sensing operation 310a.

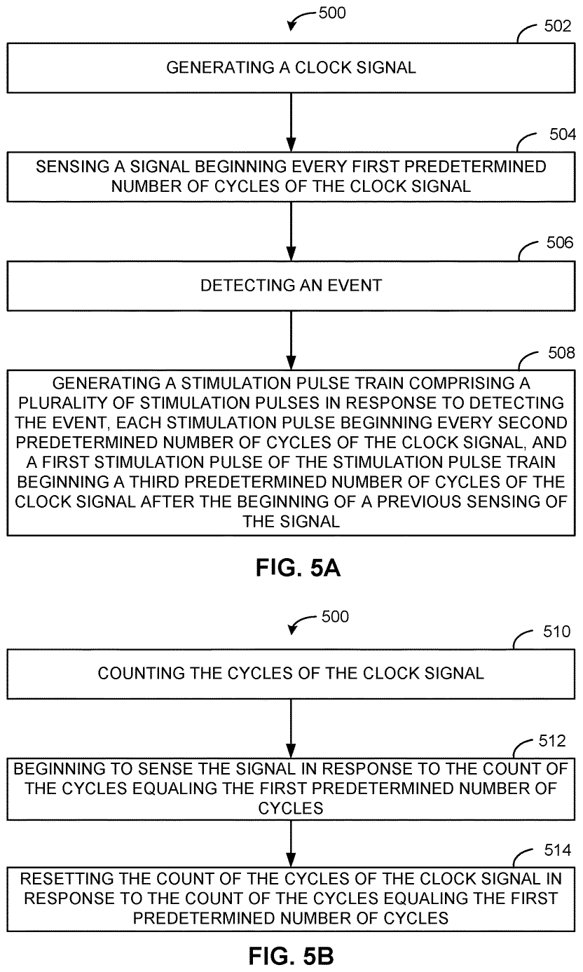

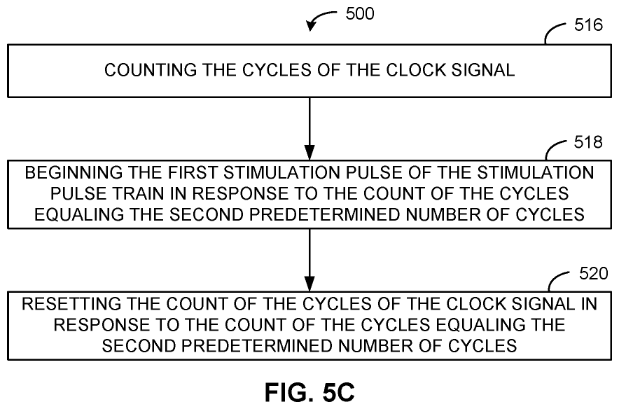

FIGS. 5A-5C are flow diagrams illustrating one example of a method 500 for sensing and applying stimulation in a timed relationship relative to each other. As illustrated in FIG. 5A at 502, method 500 includes generating a clock signal (e.g., via clock 202 of FIGS. 2A-2B). At 504, method 500 includes sensing a signal (e.g., via sensing circuit 204 of FIGS. 2A-2B) beginning every first predetermined number of cycles (e.g., 404 of FIG. 4) of the clock signal. At 506, method 500 includes detecting an event (e.g., via event detector 206 of FIGS. 2A-2B). At 508, method 500 includes generating a stimulation pulse train (e.g., via stimulation circuit 208 of FIGS. 2A-2B) comprising a plurality of stimulation pulses (e.g., 308 of FIGS. 3A-4) in response to detecting the event, each stimulation pulse beginning every second predetermined number of cycles (e.g., 406 of FIG. 4) of the clock signal, and a first stimulation pulse of the stimulation pulse train beginning a third predetermined number of cycles (e.g., 408 of FIG. 4) of the clock signal after the beginning of a previous sensing of the signal.

As illustrated in FIG. 5B at 510, method 500 may further include counting the cycles of the clock signal (e.g., via counter 220 of FIG. 2B). At 512, method 500 may further include beginning to sense the signal (e.g., via sensing circuit 204 of FIG. 2B) in response to the count of the cycles equaling the first predetermined number of cycles. At 514, method 500 may further include resetting the count of the cycles of the clock signal in response to the count of the cycles equaling the first predetermined number of cycles.

As illustrated in FIG. 5C at 516, method 500 may further include counting the cycles of the clock signal (e.g., via counter 222 of FIG. 2B). At 518, method 500 may further include beginning the first stimulation pulse of the stimulation pulse train (e.g., via stimulation circuit 208 of FIG. 2B) in response to the count of the cycles equaling the second predetermined number of cycles. At 520, method 500 may further include resetting the count of the cycles of the clock signal in response to the count of the cycles equaling the second predetermined number of cycles.

In one example, the first predetermined number equals the second predetermined number (e.g., as shown in FIGS. 3A and 4). In other examples, the first predetermined number is an integer multiple (e.g., as shown in FIG. 3B) or an integer divisor (e.g., as shown in FIG. 3C) of the second predetermined number as previously described.

In some examples, the example devices and/or example methods described in association with FIGS. 2A-5C may be performed, implemented, etc. via at least some of substantially the same features and attributes, or may comprise an example implementation of, the examples described in association with at least FIGS. 1A-1C and FIGS. 6-10C.

FIGS. 6-9 are diagrams schematically representing example arrangements (e.g., example devices and/or example methods) 700, 720, 730, 750, 800, 1300 for sensing and/or applying stimulation. In some examples, these arrangements may comprises at least some of substantially the same features and attributes as (and/or an example implementation of) the examples described in association with at least FIGS. 1A-5C and/or FIGS. 10A-10C.

With this in mind, the example arrangement 700 in FIG. 6 comprises a first implantable stimulation lead 702 including a first stimulation element 704, which comprises a plurality of spaced apart electrodes 706, with stimulation lead 702 being chronically implanted within a patient's body. In some examples, the various electrodes 706 of stimulation element 704 may be used to deliver a stimulation signal to target tissue. In some such examples, at least some of the electrodes 706 also may be used for sensing within the patient's body. Moreover, via this example arrangement, timing may be coordinated between such sensing and stimulation performed via and among electrodes 706.

In some examples, the example arrangement 700 also may comprise a second implantable stimulation lead 712 including a second stimulation element 714, which comprises a plurality of spaced apart electrodes 716. In a manner similar for first stimulation lead 702, timing may be coordinated between sensing and stimulation performed via and among electrodes 716.

In addition, in some examples, both the first and second stimulation leads 702, 712 may be implanted in a manner in which sensing may be performed using at least one electrode 706 of the first stimulation lead 702 and at least one electrode 716 of the second stimulation lead 712 and/or in which stimulation may be performed using at least one electrode 706 of the first stimulation lead 702 and at least one electrode 716 of the second stimulation lead 712. Via this arrangement, timing may be coordinated between sensing and stimulation performed via and among such electrodes 706, 716. In some such examples, the first stimulation lead 702 may be implanted on a first side (e.g., left side) of the patient's body while the second stimulation lead 712 may be implanted on a second side (e.g., right side) of the patient's body to enable bilateral stimulation and/or sensing across the patient's body (or sensing on one side of the body), as desired, with timing being coordinated between such sensing and stimulation. At least some of the various types of such sensing are described in association with at least FIGS. 1A-2C and/or FIG. 9.

FIG. 7A illustrates another example arrangement 720 (e.g., example device and/or example method) for sensing and/or applying stimulation. As shown in FIG. 7A, the example arrangement 720 may comprise a stimulation lead 722 like stimulation lead 702 of FIG. 6, except further comprising a dedicated sensor(S) 725. The dedicated sensor 725 may comprise any one of a wide variety of sensors such as, but not limited to, a pressure sensor, a sensor for sensing body position, motion, activity and the like, or other type of sensor. In some examples, the dedicated sensor 725 may comprise an electrode which is dedicated for sensing.

FIG. 7B illustrates another example arrangement 730 (e.g., example device and/or example method) for sensing and/or applying stimulation. As shown in FIG. 7B, the example arrangement 730 may comprise a stimulation lead 732 like stimulation lead 722 of FIG. 7A, except further comprising the dedicated sensor(S) 725 not being supported by the lead 732. Rather, dedicated sensor (S) 725 may be implanted within the patient's body in a location suitable to sense a desired physiologic phenomenon, which may or may not be in close proximity to the implanted location of the stimulation element 704.

FIG. 8A illustrates an example stimulation element 750 for sensing and/or applying stimulation in a manner similar to that shown and described in association with FIGS. 6-7B, except comprising a plurality of electrodes 756 arranged in a grid pattern (e.g., 2×3, 3×3, 3×4, etc.) on a carrier body 754. In a manner similar to that described for at least FIG. 6, the various electrodes 756 may be used for sensing and/or stimulation in desired combinations with timing of such sensing and stimulation being coordinated according to at least the examples of FIGS. 2A-5C of the present disclosure.

FIG. 8B is a diagram of an example arrangement comprising at least some of substantially the same features and attributes as the example arrangements in FIGS. 1A-8A, with at least some various example sensors forming part of an implantable medical device (IMD) 1333 and/or being independent of the IMD 1333 but in communication with the IMD 1333. In general terms, the sensors described in association with FIG. 9 may comprise any one or more of the sensing types, modalities, parameters, etc. as described in association with at least FIGS. 1A-8A and the example arrangement 1300 may comprise one example implementation of at least some aspects of the care engine 800 (FIG. 9) and/or example control portions 900, 920, etc. in FIGS. 10A-19C, as described later.

In some examples, the IMD 1333 may comprise an implantable pulse generator (IPG) which may form part of and/or be connected to a stimulation element with the IPG generating stimulation signals to be delivered via the stimulation element for stimulating target tissues. In some such examples, the IMD 1333 may be sized and/or shaped to be implanted and deployed as a microstimulator.

In some examples IMD 1333 may comprise an on-board sensor 1360 which is incorporated within a housing of the IMD 1333 and/or is exposed on an external surface of the housing of the IMD 1333. In some examples, the sensor 1360 may comprise an accelerometer, gyroscope, etc. to sense a wide variety of physiologic information as previously described in association with at least FIGS. 1A-8A.

In some examples, this sensed information may comprise sensed respiration, which may be used for timing application of stimulation to treat sleep disordered breathing, to evaluate the severity of the sleep disordered breathing or other disease burdens, the effectiveness of the stimulation therapy, and/or other physiologic information.

In some examples, the on-board sensor 1360 may comprise an electrode located on the external surface of a housing of the IMD 1333, and may be used for sensing physiologic information in combination with other implanted sensors, such as but not limited to electrodes 1368A, 1368B or another electrode 1361 located on the external surface of the IMD 1333. Depending on the region of the body in which the IMD 1333 and/or other electrodes (e.g., 1368A, 1368B) are implanted, in some examples the combination of electrodes may be used to sense biopotential information such as (but not limited to) electrocardiography (ECG) information, electroencephalogy (EEG) information, electromyography (EMG) information, electroneurogram (ENG), impedance, etc.

As further shown in FIG. 8B, in some example implementations the example arrangement 1300 may comprise a lead 1364 connected to and extending from the IMD 1333. The lead 1364 may comprise an element (Z) 1366 which may comprise a sensor and/or a stimulation element. In some examples, the element (Z) 1366 may comprise an electrode arrangement via which sensing and/or stimulation may be performed. In some examples, the element (Z) 1366 may comprise a dedicated sensing element and/or a dedicated stimulation element (e.g., electrode(s)).

It will be understood that the on-board sensor 1360 may comprise multiple types of sensors, at least some of which are described above, such as but not limited to accelerometer(s), etc. In some examples in which the on-board sensor 1360 is implemented, the lead 1364 may be omitted such that the IMD 1333 may comprise a leadless sensing arrangement.

In some examples, the example arrangement 1300 may be implemented in association with and/or via at least some external sensors relating to at least some of the sensing types, modalities, physiologic parameters, etc. which were described above as being implemented via implantable sensors.

It will be understood that the various sensing elements 110 and/or stimulation elements 120 (FIGS. 1A-1C, FIGS. 6-8B) may be deployed within the various regions of the patient's body to sense and/or otherwise diagnose, monitor, treat various physiologic conditions such as, but not limited to those examples described below in association with at least care engine 800 in FIG. 9 and/or as previously described in association with at least FIGS. 1A-8B.

FIG. 9 is a block diagram schematically representing an example care engine 800. In some examples, the care engine 800 may form part of a control portion 900 (FIG. 10A), such as but not limited to comprising at least part of the instructions 911. In some examples, the care engine 800 may be used to implement at least some of the various example devices and/or example methods of the present disclosure as previously described in association with FIGS. 1A-8B and/or in later described examples devices and/or methods. In some examples, the care engine 800 and/or control portion 900 (FIG. 10A) may form part of, and/or be in communication with, the example arrangements, sensing elements, stimulation elements, leads, microstimulators, pulse generators, etc. such as a portion of the devices and methods described in association with at least FIGS. 1A-8B and/or the later described examples. It will be understood that various sub-engines, functions, parameters, etc. of care engine 800 may be operated interdependently and/or in coordination with each other, in at least some examples.

In some examples, as shown in FIG. 9, the care engine 800 may comprise a sensing sub-engine 802 to track and/or control sensing of, or at, physiologic phenomenon (e.g., in a patient's body), such as described in association with FIGS. 1A-8B. Care engine 800 also may comprise a stimulation sub-engine 804 to track and/or control implementation of stimulation via a stimulation signal, such as described in association with FIGS. 1A-8B, and may comprise a physiologic system sub-engine to facilitate sensing and/or stimulation (via 802, 804) for one or more physiologic systems of the patient's body.

In some examples, the stimulation sub-engine 804 comprises a closed loop parameter 812, an open loop parameter 814, and/or a combination parameter 816 comprising aspects of both open loop stimulation and closed-loop stimulation.

In some examples, via the closed loop parameter 812, the stimulation sub-engine 804 may track and/or control stimulation of a target tissue according to a closed loop protocol in which stimulation is delivered relative to (e.g., based on, triggered by, timed with, etc.) a sensed parameter, such as some physiologic information sensed via sensing sub-engine 802 and any one or more of the sensors of the examples of the present disclosure. In this context, the sensed parameter may sometimes be referred to as providing sensed feedback to the delivered stimulation.

In some examples, via the open loop parameter 814, the stimulation sub-engine 804 may track and/or control stimulation of a target tissue according to an open loop protocol in which stimulation is delivered independent of (e.g., not based on, not triggered by, not in response to etc.) a sensed parameter.

Further details regarding both the closed loop and open loop parameters 812, 814 are described below.

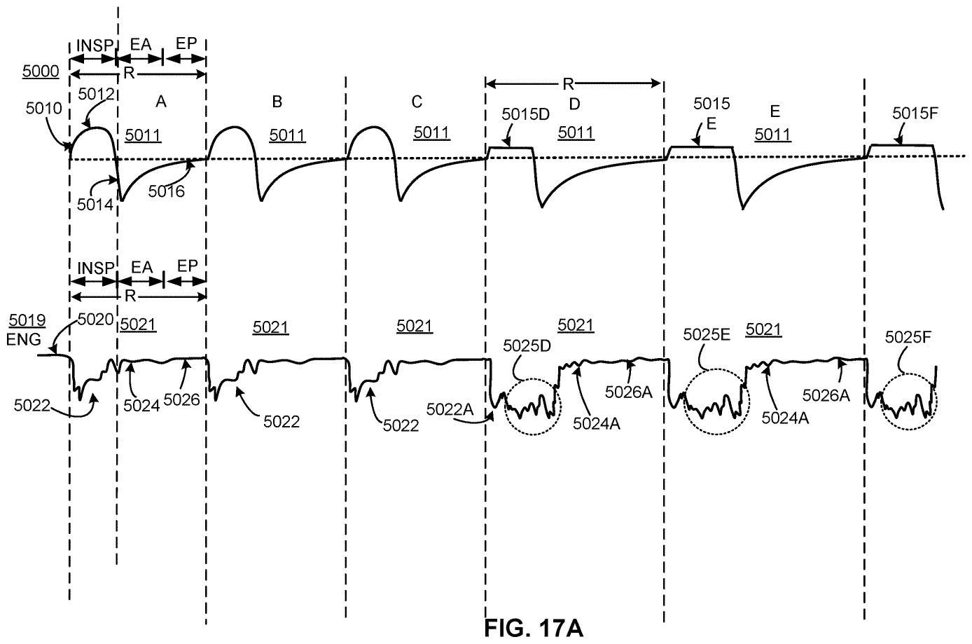

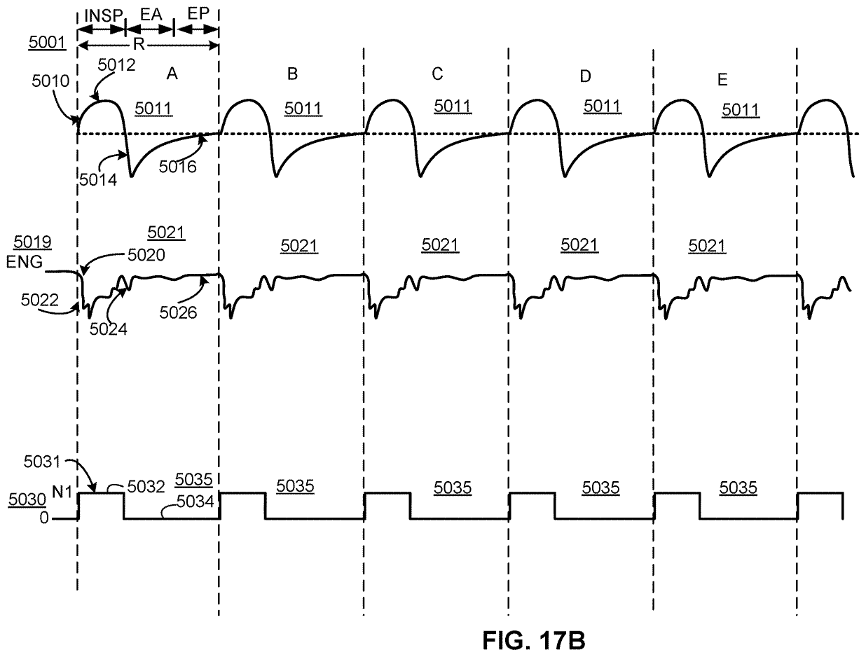

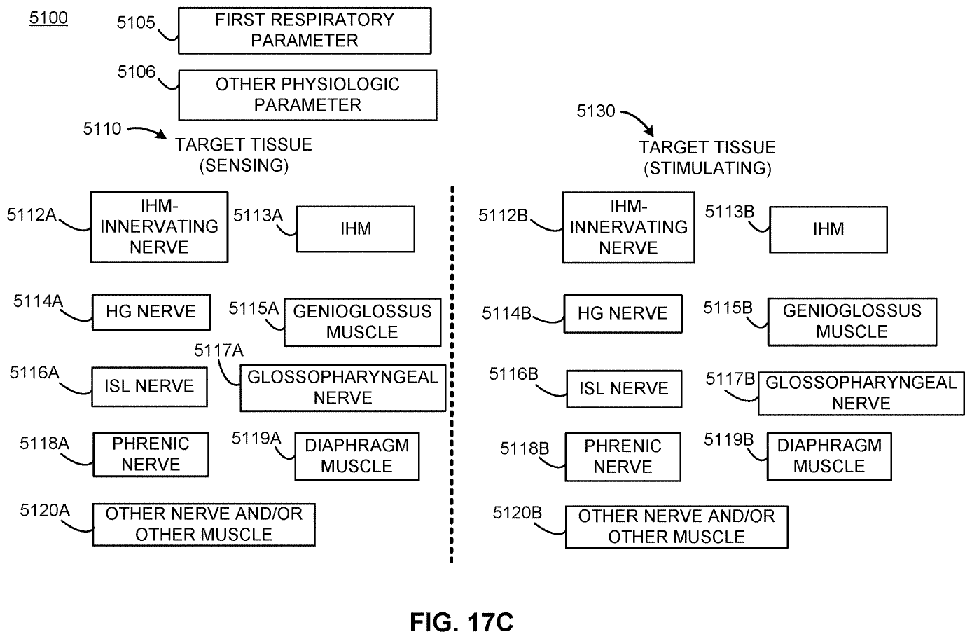

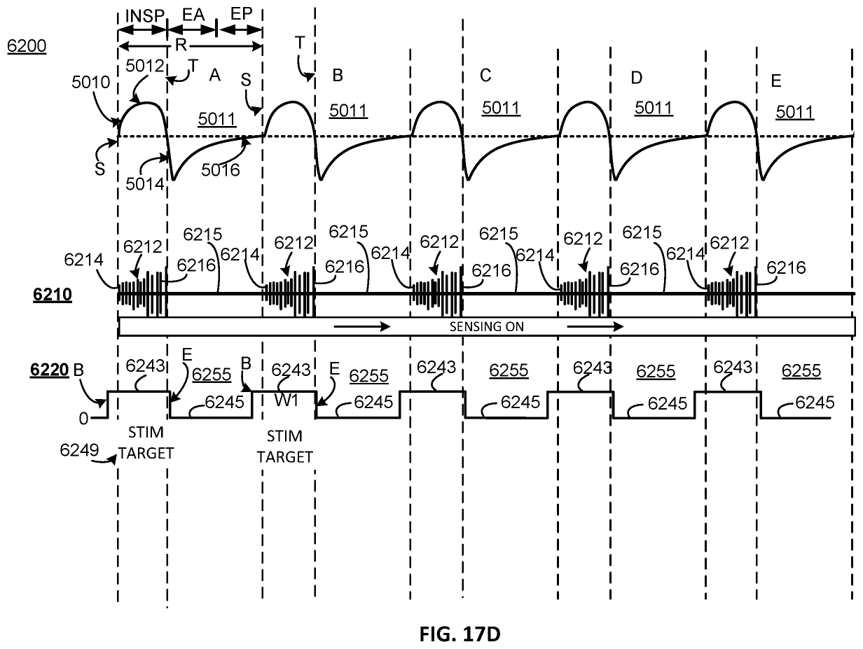

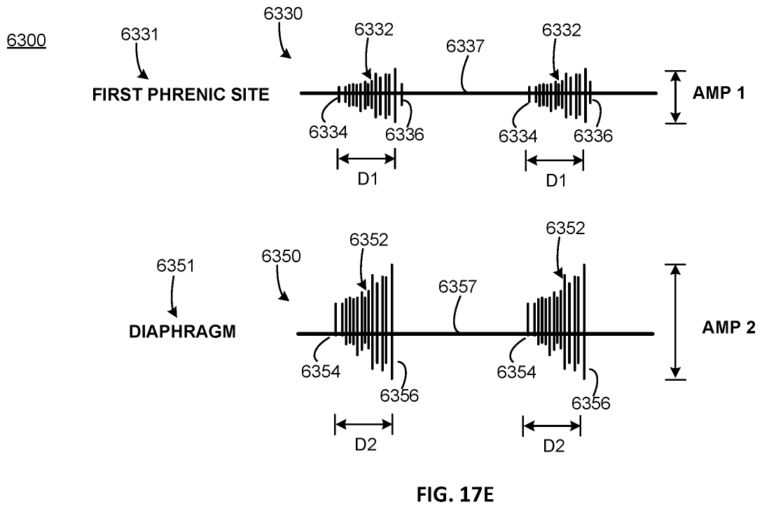

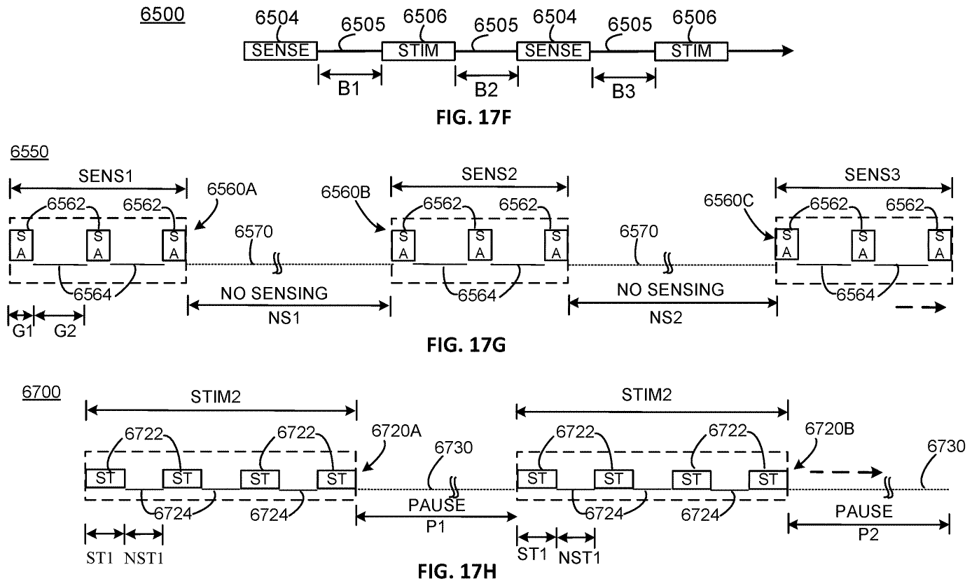

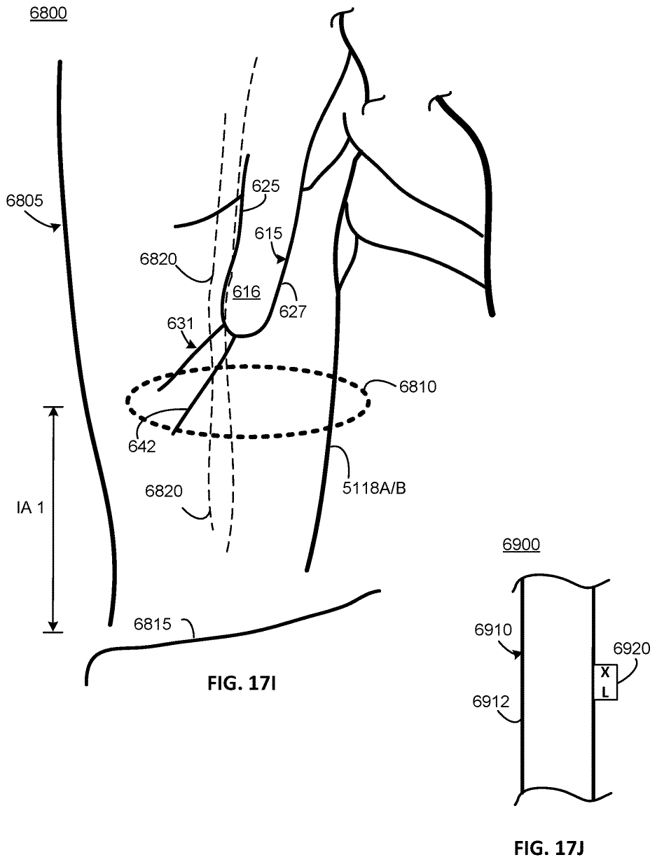

With regard to the various examples of the present disclosure, in some examples, delivering stimulation to target tissues such as an upper airway patency-related motor nerve (e.g., hypoglossal, IHM-innervating nerve) via a stimulation element (e.g., 120 in FIG. 1B-1C) is to cause contraction of upper airway patency-related muscles, which may cause or maintain opening of the upper airway to prevent and/or treat obstructive sleep apnea. Similarly, such electrical stimulation may be applied to a phrenic nerve via the stimulation element 120 to cause contraction of the diaphragm as part of preventing or treating at least central sleep apnea. As later described in association with at least FIGS. 11-26 (e.g., particularly FIGS. 17A-17J), in some examples sensing and/or stimulation of the phrenic nerve (and/or diaphragm muscle) may be used to facilitate stimulation therapy regarding respiration, including treating various forms of sleep disordered breathing. It will be further understood that some example methods may comprise treating both obstructive sleep apnea and central sleep apnea, such as but not limited to, instances of multiple-type sleep apnea in which both types of sleep apnea may be present at least some of the time. In some such instances, separate stimulation leads may be provided or a single stimulation lead may be provided but with a bifurcated distal portion with each separate distal portion extending to a respective one of the upper airway patency-related motor nerve (e.g., hypoglossal nerve, IHM-innervating nerve) and the phrenic nerve. In some examples, one of the stimulation leads may be used to stimulate other nerves such as (but not limited to) the iSL nerve, afferent nerve fibers/branches of the glossopharyngeal nerve, and/or other sensory nerves, which when stimulated, may elicit (via the CNS) a reflex opening response which activates at least some of the above-identified nerves and/or muscles to facilitate respiration to prevent and/or overcome sleep disordered breathing, as further described below in association with at least FIGS. 11-26.

In some such examples, the contraction of the upper airway patency-related motor nerve and/or contraction of other nerve (e.g., phrenic nerve) caused by electrical stimulation comprises a suprathreshold stimulation, which is in contrast to a subthreshold stimulation (e.g., mere tone) of such muscles. In one aspect, a suprathreshold intensity level corresponds to a stimulation energy greater than the nerve excitation threshold, such that the suprathreshold stimulation may provide for higher degrees (e.g., maximum, other) upper-airway clearance (i.e., patency) and sleep apnea therapy efficacy.

In some examples, a target intensity level of stimulation energy is selected, determined, implemented, etc. without regard to intentionally establishing a discomfort threshold of the patient (such as in response to such stimulation). Stated differently, in at least some examples, a target intensity level of stimulation may be implemented to provide the desired efficacious therapeutic effect in reducing sleep disordered breathing (SDB) without attempting to adjust or increase the target intensity level according to (or relative to) a discomfort threshold.

In some examples, the treatment period (during which stimulation may be applied at least part of the time) may comprise a period of time beginning with the patient turning on the therapy device and ending with the patient turning off the device. In some examples, the treatment period may comprise a selectable, predetermined start time (e.g., 10 p.m.) and selectable, predetermined stop time (e.g., 6 a.m.). In some examples, the treatment period may comprise a period of time between an auto-detected initiation of sleep and auto-detected awake-from-sleep time. With this in mind, the treatment period corresponds to a period during which a patient is sleeping such that the stimulation of the upper airway patency-related motor nerve and/or central sleep apnea-related nerve is generally not perceived by the patient and so that the stimulation coincides with the patient behavior (e.g., sleeping) during which the sleep disordered breathing behavior (e.g., central or obstructive sleep apnea) would be expected to occur.

In some examples the initiation or termination of the treatment period may be implemented automatically based on sensed sleep state information, which in turn may comprise sleep stage information.

To avoid enabling stimulation prior to the patient falling asleep, in some examples stimulation can be enabled after expiration of a timer started by the patient (to enable therapy with a remote control), or enabled automatically via sleep stage detection. To avoid continuing stimulation after the patient wakes, stimulation can be disabled by the patient using a remote control, or automatically via sleep stage detection. Accordingly, in at least some examples, these periods may be considered to be outside of the treatment period or may be considered as a startup portion and wind down portion, respectively, of a treatment period.

In some examples, stimulation of an upper airway patency-related motor nerve may be performed via open loop stimulation, such as via open loop parameter 814 of stimulation sub-engine 1404 (FIG. 9). In some examples, the open loop stimulation may refer to performing stimulation without use of any sensory feedback of any kind relative to the stimulation.

In some examples, the open loop stimulation may refer to stimulation performed without use of sensory feedback by which timing of the stimulation (e.g., synchronization) would otherwise be determined relative to respiratory information (e.g., respiratory cycles). However, in some such examples, some sensory feedback may be utilized to determine, in general, whether the patient should receive stimulation based on a severity of sleep apnea behavior and/or based on other parameters.

Conversely, in some examples and as previously described in relation to at least several examples, stimulation of an upper airway patency-related motor nerve may be performed via closed loop stimulation, such as via parameter 812 of stimulation sub-engine 804 (FIG. 9). In some examples, the closed loop stimulation may refer to performing stimulation relative to (based on, triggered by, timed according to, and the like) sensory feedback regarding parameters of the stimulation and/or effects of the stimulation.

In some examples, the closed loop stimulation may refer to stimulation performed via use of sensory feedback by which timing of the stimulation (e.g., synchronization) is determined relative to respiratory information, such as but not limited to respiratory cycle information, which may comprise onset, offset, duration, magnitude, morphology, etc. of various features of the respiratory cycles, including but not limited to the inspiratory phase, expiratory active phase, etc. In some examples, the respiration information excludes (i.e., is without) tracking a respiratory volume and/or respiratory rate. In some examples, stimulation based on such synchronization may be delivered throughout a treatment period or throughout substantially the entire treatment period. In some examples, such stimulation may be delivered just during a portion or portions of a treatment period.

In some examples of “synchronization”, synchronization of the stimulation relative to the inspiratory phase may extend to a pre-inspiratory period and/or a post-inspiratory phase. For instance, in some such examples, a beginning of the synchronization may occur at a point in each respiratory cycle which is just prior to an onset of the inspiratory phase. In some examples, this point may be about 200 milliseconds, or 300 milliseconds prior to an onset of the inspiratory phase.

In some examples in which the stimulation is synchronous with at least a portion of the inspiratory phase, the upper airway muscles are contracted via the stimulation to ensure they are open at the time the respiratory drive controlled by the central nervous system initiates an inspiration (inhalation). In some such examples, in combination with the stimulation occurring during the inspiratory phase, example implementation of the above-noted pre-inspiratory stimulation helps to ensure that the upper airway is open before the negative pressure of inspiration within the respiratory system is applied via the diaphragm of the patient's body. In one aspect, this example arrangement may minimize the chance of constriction or collapse of the upper airway, which might otherwise occur if flow of the upper airway flow were too limited prior to the full force of inspiration occurring.

In some such examples, the stimulation of the upper airway patency-related motor nerve may be synchronized to occur with at least a portion of the expiratory period.

With regard to at least the methods of treating sleep apnea as previously described in association with at least FIGS. 1A-9, at least some such methods may comprise performing the delivery of stimulation to the upper airway patency-related first (motor) nerve without synchronizing such stimulation relative to a portion of a respiratory cycle. In some instances, such methods may sometimes be referred to as the previously described open loop stimulation.

In some examples, the term “without synchronizing” may refer to performing the stimulation independently of timing of a respiratory cycle. In some examples, the term “without synchronizing” may refer to performing the stimulation while being aware of respiratory information but without necessarily triggering the initiation of stimulation relative to a specific portion of a respiratory cycle or without causing the stimulation to coincide with a specific portion (e.g., inspiratory phase) of a respiratory cycle.

In some examples, in this context the term “without synchronizing” may refer to performing stimulation upon the detection of sleep disordered breathing behavior (e.g., obstructive sleep apnea events) but without necessarily triggering the initiation of stimulation relative to a specific portion of a respiratory cycle or without causing the stimulation to coincide with the inspiratory phase. At least some such examples may be described in Wagner et al., STIMULATION FOR TREATING SLEEP DISORDERED BREATHING, published as US 2018/0117316 on May 3, 2018, and which is incorporated by reference herein in its entirety.

In some examples, while open loop stimulation may be performed continuously without regard to timing of respiratory information (e.g., inspiratory phase, expiratory phase, etc.) such an example method and/or system may still comprise sensing respiration information for diagnostic data and/or to determine whether (and by how much) the continuous stimulation should be adjusted. For instance, via such respiratory sensing, it may be determined that the number of sleep disordered breathing (SDB) events are too numerous (e.g., an elevated AHI) and therefore the intensity (e.g., amplitude, frequency, pulse width, etc.) of the continuous stimulation should be increased or that the SDB events are relatively low such that the intensity of the continuous stimulation can be decreased while still providing therapeutic stimulation. It will be understood that via such respiratory sensing, other SDB-related information may be determined which may be used for diagnostic purposes and/or used to determine adjustments to an intensity of stimulation, initiating stimulation, and/or terminating stimulation to treat sleep disordered breathing. It will be further understood that such “continuous” stimulation may be implemented via selectable duty cycles, train of stimulation pulses, selective activation of different combinations of electrodes, etc.

In some examples of open loop stimulation or closed loop stimulation, some sensory feedback may be utilized to determine, in general, whether the patient should receive stimulation based on a severity of sleep apnea behavior. In other words, upon sensing that a certain number of sleep apnea events are occurring, the device may implement stimulation.

Some non-limiting examples of such devices and methods to recognize and detect the various features and patterns associated with respiratory effort and flow limitations include, but are not limited to: Dieken et al., RESPIRATION DETECTION, published as WO/2021/016562 on Jan. 28, 2021; Christopherson et al., U.S. Pat. No. 8,938,299, SYSTEM FOR TREATING SLEEP DISORDERED BREATHING, issued Jan. 20, 2015; Christopherson et al., U.S. Pat. No. 5,944,680, titled RESPIRATORY EFFORT DETECTION METHOD AND APPARATUS; and Testerman, U.S. Pat. No. 5,522,862, titled METHOD AND APPARATUS FOR TREATING OBSTRUCTIVE SLEEP APNEA, all of which are hereby incorporated by reference.

Moreover, in some examples various stimulation methods may be applied to treat obstructive sleep apnea, which include but are not limited to: Ni et al., SYSTEM FOR SELECTING A STIMULATION PROTOCOL BASED ON SENSED RESPIRATORY EFFORT, which issued as U.S. Pat. No. 10,583,297 on Mar. 10, 2020; Christopherson et al., U.S. Pat. No. 8,938,299, SYSTEM FOR TREATING SLEEP DISORDERED BREATHING, issued Jan. 20, 2015; and Wagner et al., STIMULATION FOR TREATING SLEEP DISORDERED BREATHING, published as US 2018/0117316 on May 3, 2018, each of which is hereby incorporated by reference herein in its entirety.

As shown in FIG. 9, in some examples, the physiologic system sub-engine 860 is to track and/or control sensing and/or stimulation in relation to one or more physiologic systems such as, but not limited to, a respiratory system 863, an upper airway system 864, a pelvic system 865, and/or other physiologic system 869.

In some examples, the tracking and/or the controlling of sensing and/or stimulation for the respiratory system 863 and/or upper airway system 864 (e.g., as part of the respiratory system 863) may comprise such sensing and/or stimulation related to care (e.g., diagnose, monitor, treat, etc.) for sleep disordered breathing such as, but not limited to, obstructive sleep apnea, central sleep apnea, or multiple-type apnea. In some such examples, stimulation may comprise applying stimulation to an upper airway patency-related motor nerve such as, but not limited to, a hypoglossal nerve, IHM-innervating nerve and/or other nerves or muscles which contribute to upper airway patency. In some such examples, stimulation of the hypoglossal nerve and/or other nerves may contribute to at least protrusion of the tongue to enhance upper airway patency. In some examples, stimulation of such nerves (and/or muscles) may enhance upper airway patency by contracting muscles other than the tongue.

In some examples, the tracking and/or the controlling of sensing and/or stimulation for the pelvic system 865 may comprise such sensing and/or stimulation related to care (e.g., diagnosing, monitoring, treatment, etc.) for pelvic dysfunctions such as, but not limited to, urinary incontinence (e.g., stress, other), fecal incontinence, and so on. In some such examples, the stimulation may comprise electrical stimulation of body tissues, which control contraction of an external urinary sphincter, an external anal sphincter, etc. In some examples, the body tissues may comprise a nerve(s), a muscle(s), and/or both nerve(s) and muscle(s). Some example nerves comprise a pudendal nerve, such as the pudendal nerve trunk or deep perineal branch of the pudendal nerve, among other nerves including the hypogastric nerve and pelvic splanchnic nerves. Some example muscles comprise at least those muscles innervated by the above-named nerves and/or other muscles.

In some examples, at least one other physiologic system to be sensed may comprise a cardiac system. The tracking and/or the controlling of sensing and/or stimulation for the cardiac system (and related bodily systems, functions, etc.) may comprise such sensing and/or stimulation related to care (e.g., diagnosing, monitoring, treatment, etc.) of cardiac conditions such as, but not limited to, cardiac arrhythmias, atrial fibrillation, ventricular fibrillation, and the like. In some such examples, such sensing and/or stimulation may be associated with sensing and/or stimulation involving the respiratory system 863, upper airway system 864, and/or other physiologic system.

In some examples, the care engine 800 may comprise a sleep disordered breathing (SDB) sub-engine 880 which can track and/or control sensing and/or stimulation related to care (e.g., diagnosing, monitoring, treatment, etc.) for sleep disordered breathing such as, but not limited to, obstructive sleep apnea, central sleep apnea, or multiple-type apnea. In some examples, the sleep disordered breathing sub-engine 880 may operate in cooperation with, or a complementary manner, with at least the respiratory 863 and/or upper airway 864 systems of physiologic systems sub-engine 860. In some examples, the SDB sub-engine 880 may track and/or control sensing and/or stimulation in relation to SDB-related parameters such as, but not limited to SDB events 881, sleep-wake detection or status 882, respiration detection 883, other SDB parameters 884, and/or the like. In some examples, SDB events parameter 881 (or other physiologic events) may be identified and/or implemented via at least some of substantially the same features and attributes as described in Dieken et al., DISEASE BURDEN INDICATION, filed as PCT Application PCT/US21/042601 on Jul. 21, 2021.

In some examples, sleep-wake detection or status parameter 882 may be identified and/or implemented via at least some of substantially the same features and attributes as described in Rondoni et al., SLEEP DETECTION FOR SLEEP DISORDERED BREATHING (SDB) CARE, published as PCT Publication WO/2021/016558 on Jan. 28, 2021.

In some examples, respiration detection parameter 883 may be identified and/or implemented via at least some of substantially the same features and attributes as described in Dieken et al., RESPIRATION DETECTION, published as PCT Publication WO/2021/016562 on Jan. 28, 2021.

FIG. 10A is a block diagram schematically representing an example control portion 900. In some examples, control portion 900 provides one example implementation of a control portion forming a part of, implementing, and/or generally managing the sensing elements, stimulation elements, sensing circuits, stimulation circuits, clocks, pulse generators, devices, user interfaces, instructions, information, engines, sub-engines, functions, actions, and/or methods, as described throughout examples of the present disclosure in association with FIGS. 1A-9.

In some examples, control portion 900 includes a controller 902 and a memory 910. In general terms, controller 902 of control portion 900 comprises at least one processor 904 and associated memories. The controller 902 is electrically couplable to, and in communication with, memory 910 to generate control signals to direct operation of at least some of sensing elements, stimulation elements, sensing circuits, stimulation circuits, clocks, pulse generators, devices, user interfaces, instructions, information, engines, sub-engines, elements, functions, actions, and/or methods, as described throughout examples of the present disclosure. In some examples, these generated control signals include, but are not limited to, employing instructions 911 and/or information stored in memory 910 to at least direct and manage sensing, stimulation signals, and timing the sensing and the stimulation relative to each other, among other related aspects, as described throughout the examples of the present disclosure in association with FIGS. 1A-9. In some such examples, this sensing, stimulation, and their relative timing, may be used in treatment of sleep disordered breathing such as obstructive sleep apnea and/or central sleep apnea, sensing physiologic information including but not limited to respiratory information, heart rate, and/or monitoring sleep disordered breathing, etc. In some such examples, the sensing, stimulation, and/or their relative timing may be used in treatment of pelvic dysfunction, cardiac dysfunction, or other conditions. In some instances, the controller 902 or control portion 900 may sometimes be referred to as being programmed to perform the above-identified actions, functions, etc. In some examples, at least some of the stored instructions 911 are implemented as, or may be referred to as, a care engine (e.g., 800 in FIG. 9). In some examples, at least some of the stored instructions 911 and/or information may form at least part of, and/or, may be referred to as a care engine.

In response to or based upon commands received via a user interface (e.g., user interface 940 in FIG. 10C) and/or via machine readable instructions, controller 902 generates control signals as described above in accordance with at least some of the examples of the present disclosure. In some examples, controller 902 is embodied in a general purpose computing device while in some examples, controller 902 is incorporated into or associated with at least some of the sensing elements, stimulation elements, sensing circuits, stimulation circuits, clocks, pulse generators, devices, user interfaces, instructions, information, engines, sub-engines, functions, actions, and/or methods, etc. as described throughout examples of the present disclosure.

For purposes of this application, in reference to the controller 902, the term “processor” shall mean a presently developed or future developed processor (or processing resources) that executes machine readable instructions contained in a memory. In some examples, execution of the machine readable instructions, such as those provided via memory 910 of control portion 900 cause the processor to perform the above-identified actions, such as operating controller 902 to implement the apnea treatment as generally described in (or consistent with) at least some examples of the present disclosure. The machine readable instructions may be loaded in a random access memory (RAM) for execution by the processor from their stored location in a read only memory (ROM), a mass storage device, or some other persistent storage (e.g., non-transitory tangible medium or non-volatile tangible medium), as represented by memory 910. In some examples, the machine readable instructions may comprise a sequence of instructions, a processor-executable machine learning model, or the like. In some examples, memory 910 comprises a computer readable tangible medium providing non-volatile storage of the machine readable instructions executable by a process of controller 902. In some examples, the computer readable tangible medium may sometimes be referred to as, and/or comprise at least a portion of, a computer program product. In other examples, hard wired circuitry may be used in place of or in combination with machine readable instructions to implement the functions described. For example, controller 902 may be embodied as part of at least one application-specific integrated circuit (ASIC), at least one field-programmable gate array (FPGA), and/or the like. In at least some examples, the controller 902 is not limited to any specific combination of hardware circuitry and machine readable instructions, nor limited to any particular source for the machine readable instructions executed by the controller 902.

In some examples, control portion 900 may be entirely implemented within or by a stand-alone device.

In some examples, the control portion 900 may be partially implemented in one of the example arrangements (or portions thereof) and partially implemented in a computing resource separate from, and independent of, the example arrangements (or portions thereof) but in communication with the example arrangements (or portions thereof). For instance, in some examples, control portion 900 may be implemented via a server accessible via the cloud and/or other network pathways. In some examples, the control portion 900 may be distributed or apportioned among multiple devices or resources, such as among a server, an example sensing circuit, example stimulation circuit, and/or clock, and/or a user interface.

In some examples, control portion 900 includes, and/or is in communication with, a user interface 940 as shown in FIG. 10C and described below.

FIG. 10B is a diagram schematically illustrating an example arrangement 920 of at least some example implementations by which the control portion 900 (FIG. 10A) can be implemented, according to one example of the present disclosure. In some examples, control portion 920 is entirely implemented within or by a pulse generator 922 (or sensing monitor), which has at least some of substantially the same features and attributes as a pulse generator (e.g., power/control element, etc.) as previously described throughout the present disclosure. In some examples, control portion 920 is entirely implemented within or by a remote control 930 (e.g., a programmer) external to the patient's body, such as a patient control 932 and/or a clinician control 934. In some examples, at least some aspects of the control portion 920 may be implemented within a portal 936, such as a web portal. In some examples, the control portion 920 may be partially implemented in the pulse generator 922 and partially implemented in the remote control 930 (at least one of patient control 932 and clinician control 934). In some examples, the remote control 930 may comprise a smart phone, tablet, smart watch, etc. or other mobile computing device.

FIG. 10C is a block diagram schematically representing user interface 940, according to one example of the present disclosure. In some examples, user interface 940 forms part of and/or is accessible via a device external to the patient and by which the therapy system may be at least partially controlled and/or monitored. The external device which hosts user interface 940 may be a patient remote (e.g., 932 in FIG. 10B), a clinician remote (e.g., 934 in FIG. 10B) and/or a portal 936. In some examples, user interface 940 comprises a user interface or other display that provides for the simultaneous display, activation, and/or operation of at least some of the various sensing elements, stimulation elements, sensing circuits, stimulation circuits, clocks, pulse generators, devices, instructions, information, engines, sub-engines functions, and/or methods, as described in association with FIGS. 1A-9. In some examples, at least some portions or aspects of the user interface 940 are provided via a graphical user interface (GUI), and may comprise a display 944 and input 942.

While at least some of the above-described examples are directed to sensing and stimulating in a timed relationship, at least some examples in accordance with the present disclosure are not so limited. For example, the devices 105, 150, 160 of FIGS. 1A-1C, devices 200a, 200b of FIGS. 2A-2B, and/or arrangements, engines, and/or control portions of FIGS. 6-10C may be used to sense a first respiration parameter from a first target tissue (e.g., IHM-innervating nerve) and/or stimulate a second target tissue (e.g., hypoglossal nerve or iSL nerve). In some examples, sensing of the first respiration parameter is timed independent of stimulating the second target tissue. For example, sensing of the first respiration parameter may occur without use of a common clock signal to time stimulation of the second target tissue, such that timing of the sensing occurs irrespective of (e.g., independent of) the stimulation of the second target tissue. In some such examples, the sensing of the first parameter from the first target tissue may occur at the same time as, at different times as, and/or overlapping time(s) as stimulation of the second target tissue.

For example, and referring back to FIG. 1A, an example device 105 may be configured to sense a first respiration parameter (or other physiologic parameter) from a first target tissue 125 and/or stimulate a second target tissue 128. In some examples, the device 105 may be configured to perform each of the sensing and stimulating. For example, the device 105 may comprise a sensing circuit 152 (FIG. 1B) to receive sensed physiologic information from sensor 110, as sensed from first target tissue 125, and a stimulation circuit 154 (FIG. 1B) to deliver a stimulation signal to the stimulation element 120 for application to second target tissue 128.

In some examples, each of the first and second target tissue 125, 128 may comprise a nerve portion(s), a muscle portion(s), a combination of nerve portion(s) and muscle portion(s), a neuromuscular junction of nerve portion(s) and muscle portion(s), and/or combinations thereof, that are of or within the environment 127. It will be understood that some forms of sensing (e.g., bioimpedance, other) may encompass tissues in addition to, and/or other than, nerves and muscles.

In some examples, the first and second target tissues 125, 128 include respiratory-related tissue, such as nerves and/or muscles. Non-limiting examples of respiratory-related tissue include an upper airway patency-related tissue (e.g., a hypoglossal nerve, an IHM-innervating nerve, and/or muscles innervated by), an upper airway reflex-related sensory nerve, a phrenic nerve (and/or diaphragmatic tissue), and/or among other nerves and/or the muscles. Example upper airway patency-related muscles may include, but are not limited to, the genioglossus muscle, such as protrusor muscles and IHMs. Some example muscles may comprise diaphragm muscles innervated by the phrenic nerve, among other muscles. Some example muscles also may comprise muscles (and their innervating nerves) which may be activated upon stimulation of upper airway reflex-related sensory nerves (e.g., iSL nerve, glossopharyngeal nerve), which when stimulated, may elicit (via the CNS) a reflex opening response which activates nerves (and their innervated muscles) to facilitate respiration to prevent and/or overcome sleep disordered breathing, as further described below in association with at least FIGS. 11-26.

In some examples, the first target tissue 125 and second target tissue 128 may include the same target, such as different portions of a single nerve (e.g., different portions of a single type of nerve, such as the iSL nerve). For example, the first target tissue 125 may comprise a first portion of a first respiratory-related tissue (e.g., first portion of IHM-innervating nerve) and the second target tissue 128 comprises a second portion of the (same) first respiratory-related tissue (e.g., second portion of the IHM-innervating nerve). In some such examples, the first respiratory tissue comprises a phrenic nerve or comprises an upper airway patency-related motor nerve, such as the hypoglossal nerve, the IHM-innervating nerve. In some of these examples, the first respiratory tissue may comprise a sensory nerve/branch (e.g., nerve with mostly or solely sensory/afferent fibers) such as an iSL nerve and/or the glossopharyngeal nerve from which a reflex opening response may be elicited, as noted above.

In some examples, the first and second target tissues 125, 128 comprise different targets. For example, the first target tissue 125 may comprise a first respiratory-related tissue (e.g., IHM-innervating nerve) and the second target tissue 128 may comprise a second respiratory-related tissue different from the first (e.g., iSL nerve). In some examples, the first target tissue 125 may comprise a first upper airway patency-related motor nerve and the second target tissue 128 may comprise a second upper airway patency-related motor nerve different from the first upper airway patency-related motor nerve, such as different combinations of the hypoglossal nerve, the IHM-innervating nerve, or other nerves. In some examples, the first target tissue 125 may comprise an upper airway patency-related motor nerve and the second target tissue 128 may comprise an upper airway reflex-related sensory nerve, such as different combinations of the hypoglossal nerve, the iSL nerve, the IHM-innervating nerve, and afferent nerve fibers/branch of the glossopharyngeal nerve. In some examples, the first and second target tissues 125, 128 are each selected from the hypoglossal nerve and the IHM-innervating nerve. In some examples, the first and second target tissues 125, 128 are each selected from the hypoglossal nerve and the iSL nerve. In some examples, the first and second target tissues 125, 128 are each selected from the IHM-innervating nerve and the iSL nerve. In some examples, the first and second target tissues 125, 128 are each selected from the hypoglossal nerve, the iSL nerve, and the IHM-innervating nerve. In some examples, the afferent nerve fibers/branch of the glossopharyngeal nerve may be stimulated instead of, and/or in addition to, the iSL nerve to elicit a reflex opening response.

In some examples, the first and second target tissues 125, 128 are each selected from: (i) the phrenic nerve (and/or diaphragm innervated by the phrenic nerve); (ii) one of the upper airway patency-related tissues (e.g., one of hypoglossal nerve, the IHM-innervating nerve, and muscles innervated by such nerves); and (iii) one of the upper airway reflex-related sensory nerves (e.g., afferent nerve fibers/branches which elicit (via CNS) a reflex opening response).

In some examples, the first target tissue 125 comprises a first muscle (e.g., IHM) and the second target tissue 128 comprises a first nerve (e.g., IHM-innervating nerve). The first muscle may be innervated by the first nerve or another nerve. In one non-limiting example, the first muscle may comprise a diaphragm muscle (e.g., sensed via EMG) and the first nerve may comprise a hypoglossal nerve.

In some examples, the first target tissue 125 comprises a first nerve (e.g., IHM-innervating nerve) and the second target tissue 128 comprises a second nerve (e.g., hypoglossal nerve). However, in some examples, the first nerve may comprise one branch of a nerve (e.g., hypoglossal nerve) and the second nerve may comprise a second/different branch of the same nerve (e.g., hypoglossal nerve). In some examples, the first target tissue 125 comprises the first nerve and the second target tissue 128 comprises a first muscle and, optionally, the second nerve. The first muscle may be innervated by the first nerve, the second nerve, or a different nerve.

In some examples, the first target tissue 125 comprises a first muscle and the second target tissue 128 comprises a first nerve. The first muscle (e.g., IHM) may be innervated by the first nerve (e.g., IHM-innervating nerve) or a different nerve (e.g., first muscle comprises a diaphragm muscle, which is innervated by the phrenic nerve).

In some examples, the first target tissue 125 comprises a first muscle (e.g., IHM) and the second target tissue 128 comprises a second muscle (e.g., genioglossus muscle). The first muscle and second muscle may include different portions of the same muscle (e.g., different portions of one IHM) or different muscles (e.g., two different IHMs or an IHM and the genioglossus muscle), and/or may be innervated by the same and/or different nerves or portions thereof.

Using any of the above-described examples, the device 105 of FIG. 1A (and/or the devices 150, 160 of FIGS. 1B-1C and/or 200a-200b of FIGS. 2A-2C) may sense the first respiration parameter from a muscle and/or from a nerve. Sensing of the first respiration parameter may be performed via various techniques, such as EMG (for muscle) and/or ENG (for nerves), in some such examples. For example, and as previously described at least in connection with FIG. 8B, the electrodes (e.g., 1368A, 1368B in FIG. 8B) of or in communication with the IMD may be used to sense biopotential information such as (but not limited to) ECG information, EEG information, EMG information, ENG information, bioimpedance, etc. In some examples, the first respiratory parameter may be sensed by sensing biopotential from mixed tissue sources, such as sensing the impedance across tissue between two different electrodes that are disposed on or proximate to different target tissues. The mixed tissues sources may include anatomical tissue other than or in addition to the nerves and/or muscles as illustrated herein.

In some examples, the sensing may be performed using at some of substantially the same features and attributes as described by: Verzal, et al., WO 2021/242633, published on Dec. 2, 2021, entitled “SINGLE OR MULTIPLE NERVE STIMULATION TO TREAT SLEEP DISORDERED BREATHING”, corresponding to U.S. National Stage Application, Ser. No. 17/926,010, filed on May 8, 2023, and published on ______ as U.S. Publication ______; and Verzal, et al., WO 2022/246320, published on Nov. 11, 2022, entitled “MULTIPLE TARGET STIMULATION THERAPY FOR SLEEP DISORDERED BREATHING”, corresponding to U.S. National Stage Application, Serial No. ______, filed on ______ and published on as U.S. Publication ______, each of which are incorporated herein by reference in their entireties for their teachings.

Similarly and using any of the above-described examples, the device 105 of FIG. 1A (and/or the devices 150, 160 of FIGS. 1B-1C and/or 200a-200b of FIGS. 2A-2C) may stimulate muscle and/or a nerve by applying a stimulation signal thereto. Stimulating the second target tissue 128 may be used for a variety of treatments, such as for treating sleep disordered breathing (SDB) by promoting upper airway patency. In some examples, the SDB may include an obstructive sleep apnea.

In some examples, the stimulation signal may comprise a sufficient strength (and/or other characteristics) to cause suprathreshold contraction of the target muscle portion, such as, but not limited to, stimulation of the hypoglossal nerve (HGN) resulting in protrusion of the tongue (e.g., genioglossus muscle), stimulation of the IHM-innervating nerve resulting in contraction of other upper airway muscle(s), and/or stimulation of various combinations of the HGN, IHM-innervating nerves. In some examples, and as further described below, stimulation of the iSL nerve (and/or glossopharyngeal nerve) may resulting in eliciting (via CNS) a reflex opening response, which includes activation of at least one upper airway patency-related motor nerve (and associated muscle), such as activating an array of upper airway patency-related muscles to provide a more comprehensive physiological response as compare to stimulating a single nerve and/or muscle (e.g., hypoglossal nerve or genioglossus muscle).

In some examples, the device 105 (FIG. 1A) may use the first respiration parameter to control and/or set the stimulation of the second target tissue 128, such as for treating SDB. For example, control of the stimulation may include setting the timing, may include setting the amplitude, and/or may include selecting the second target for the stimulation to be applied to, and based on, at least the first respiration parameter. In some examples, the timing may be set in relation to respiration, detection of a sleep disordered breathing event, and/or other physiological signals. In some examples, the device 105 on FIG. 1A may further include an event detector, such as the event detector 206 illustrated by the devices 200a, 200b of FIGS. 2A-2B. In some such examples, the device 105 includes the sensing circuit 152 (FIG. 1B) to sense a physiologic signal from the first target tissue 125 of a patient indicative of a first respiration parameter (using sensor 110), the stimulation circuit 154 (FIG. 1B) to stimulate the second target tissue 128 of the patient based on the first respiration parameter (using stimulation element 120), and an event detector (206 in FIGS. 2A-2B) to detect the first respiration parameter from the physiologic signal and, in response, to output a signal to the stimulation circuit 154 to set stimulation of the second target tissue 128. In some examples, the stimulation setting(s) (e.g., timing, duration, amplitude, selection of second target tissue 128) may be applied immediately or at a different time. For example, in response to applying the stimulation setting(s), the stimulation may be applied to the second target tissue 128 according to settings.

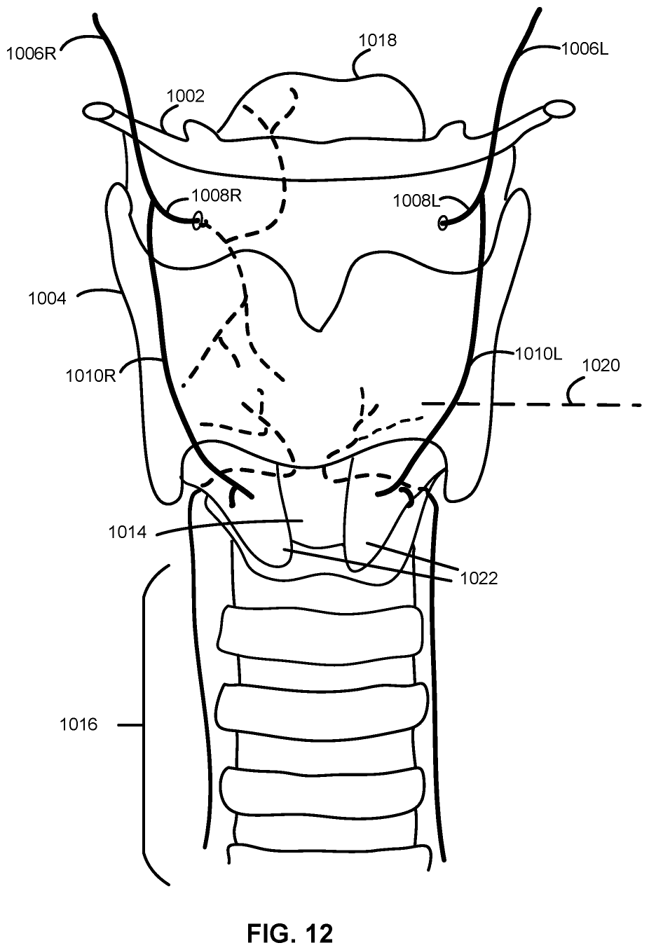

As may be appreciated, such example nerves and/or muscles may be located on both the left and right side of the patient, as illustrated herein by at least FIGS. 12, 14, and 18-21. Accordingly, in some examples, the sensing and/or the stimulating may be performed solely on one side, both sides simultaneously, and/or both sides of the patient at different times. In some examples, an example method may comprise: (i) sensing the first respiration parameter from the first target tissue 125 via bilaterally sensing the first respiration parameter from the first target tissue 125 (e.g., IHM-innervating nerve) on a first lateral side and a second lateral side of a patient; and/or (ii) stimulating the second target tissue 128 via bilaterally stimulating the second target tissue 128 (e.g., another portion of IHM-innervating nerve, IHM, or other tissue) on the first lateral side and second lateral side of the patient.

FIGS. 11-16 illustrate different example target tissue including, but not limited to, upper airway patency-related motor nerves and muscles innervated by, and upper airway reflex-related sensory nerves.

FIGS. 11 and 12 are diagrams schematically representing patient anatomy, which may be used as target tissue by an example device and/or in an example method for sensing and/or stimulating an iSL nerve, among other target tissue. The iSL nerve 1008 may include an internal branch of the superior laryngeal (SL) nerve 1006.

As shown by FIG. 11, the SL nerve 1006 extends from the inferior ganglion 1013 of the vagus nerve 1011 and with a portion (e.g., the 1010) running alongside the vagus nerve 1011 and the pharynx. The SL nerve 1006 has two branches, the iSL nerve 1008 and the external SL (eSL) nerve 1010. Among other aspects, the eSL nerve 1010 includes efferent nerve fibers (e.g., motor nerve fibers) which innervate the cricothyroid muscle 1022 (shown on both sides of the patient in FIG. 12). From the branching point 1007, the eSL nerve 1010 extends inferiorly to the thyroid cartilage 1004, and toward, the cricothyroid muscle 1022. Also shown by FIG. 11 is cricoid cartilage 1014 and the trachea 1016 of the patient.

Meanwhile, the iSL nerve 1008 includes (e.g., carries) afferent nerve fibers which extend from the laryngeal mucosa, and ultimately to the central nervous system (CNS). As shown in FIGS. 11-12A, a proximal portion of the iSL nerve 1008 may be viewed as being inferior to the hyoid bone 1002 and arising out of and through the thyrohyoid membrane 1003 (superior to the thyroid cartilage 1004) from the more distal portions of the iSL nerve 1008. As further schematically represented in FIG. 12, the more distal branches of the iSL nerve 1008 extend from the epiglottis (1018 of FIG. 12), the base of the tongue (e.g., genioglossus muscle), the epiglottis glands, and from a posterior origin in the aryepiglottic fold, from the laryngeal mucosa. Among other aspects, the laryngeal mucosa comprises mucous membrane(s) surrounding the entrance of the larynx, and the mucous lining of the larynx as far down as the vocal folds 1012.

The afferent nerve fibers of the iSL nerve 1008 may receive sensory information (which is indicative of or includes the respiratory information) from mechanoreceptors located at or near the upper airway. For example, the mechanoreceptors may form part of the tissue that the more distal branches of the iSL nerve 1008 extend from, including the epiglottis, the base of the tongue (e.g., genioglossus muscle), the epiglottis glands, the aryepiglottic fold, and/or the laryngeal mucosa.

Among other physiologic influences, in some examples, the sensed neural activity of the iSL nerve 1008 which corresponds to, and which reveals, upper airway obstruction may be associated with (and result from) mechanoreceptors located at or near the upper airway. First, it is worth noting that the mechanoreceptors may provide general respiratory information based on their behavior during the respiratory cycle. In particular, during inspiration, a contraction of the diaphragm causes negative pressure in the lungs, which induces (e.g., causes) air to enter the lungs while cells of the mechanoreceptors are stretched (and/or otherwise mechanically affected) during this inspiration. According, during regular respiration there is a baseline phasic neural activity of the mechanoreceptors which may be sensed. When an upper airway obstruction is present, an increased pressure differential is exhibited because the diaphragm may contract harder/longer in an effort to induce an adequate volume of air into the lungs, with the increased pressure differential increasing the amount of stretch on the mechanoreceptors. This increased pressure differential, in turn, causes a change in the sensory signal sent along the afferent/sensor fibers of an affected nerve (e.g., iSL nerve 1008) to the CNS, which then directs a reflex opening response to occur to overcome the obstruction. In some examples, the signal sent via afferent nerve fibers (associated with the mechanoreceptors) may convey a magnitude and/or duration of the obstruction. In some such examples, the mechanoreceptors may be in communication with and/or comprise a portion of (and/or be associated with) the iSL nerve, afferent nerve fibers/branch of the glossopharyngeal nerve, and/or other nerves.

In some examples, the second target tissue (5130 of FIG. 17C) may comprise at least some afferent nerve fibers/branches of the glossopharyngeal nerve, which may elicit a reflex opening response in a manner similar to the reflex opening response elicited via stimulation of the iSL nerve 1008.

In addition to the activation of upper airway dilator nerves/muscles, the above-noted reflex opening response also may include heightened activation of the phrenic nerve, causing increased contraction of the diaphragm muscle to enhance inspiration of air into the lungs.

Accordingly, the mechanoreceptors may sense pressure during obstruction of the upper airway, which cause a signal indicative of the sensory information to be sent to the brain via the iSL nerve 1008. The sensory information received from the afferent nerve fibers of the iSL nerve 1008, which is indicative of the sensed pressure, may be processed by the brain (e.g., CAN) to cause reflex activity include reflex opening of the upper airway. Such reflex activity may include activating different nerves (e.g., efferent nerve fibers) that innervate upper airway patency-related muscles.

In some examples, different locations of the iSL nerve 1008 may be the target tissue for sensing and/or stimulating. In some examples, the iSL nerve 1008 may be the first target tissue (125 of FIG. 1A) and/or the second target tissue (128 of FIG. 1A). In some examples, the first target tissue and second target tissue comprise the same or different portions of the iSL nerve 1008. In some examples, the first target tissue comprises the iSL nerve 1008 and the second target tissue comprises a different portion of the iSL nerve 1008. As such, in some examples, both the sensing and the stimulation of the first and second target tissues may comprise selectively sensing and stimulating afferent nerve fibers of the iSL nerve 1008. In some examples, the second target tissue may include tissue other than the iSL nerve 1008, such as the hypoglossal nerve, IHM-innervating nerve, and/or muscles innervated thereby.

In some examples, sensing the first respiratory parameter from the iSL nerve 1008 comprises sensing neural activity that is phasic with respiration. For example, neural activity may be sensed from the iSL nerve 1008, with the neural activity having an onset occurring at (or slightly preceding) the onset of inspiration and remains through the inspiratory phase of a respiratory cycle, as later further illustrated by FIGS. 17A-17B. As described above, the sensed neural activity may be associated with mechanoreceptors affected by respiration. In some examples, the neural activity may be sensed from a portion of the iSL nerve 1008 using ENG.

In some examples, as further illustrated by the timing diagrams of FIGS. 17A-17B, the neural activity may increase in amplitude and/or duty cycle as represented at 5025D, 5025E, 5028F and in response to an upper airway obstruction as represented at 5015D, 5015E, 5015F, respectively. Because the sensed neural activity is phasic with respiration (and optionally, sleep disordered breathing events), the neural activity may be used to detect respiratory information including respiration parameters of respiratory phase information. Moreover, for this same reason, increases in amplitude and/or duty cycle of the sensed neural activity may be indicative of upper airway obstruction such that the sensed neural activity may be used to detect respiratory obstruction information. By using the respiratory information sensed from the mechanoreceptors (e.g., pressure, stretch) via the iSL nerve 1008 to set stimulation, stimulation therapy may be adjusted in real time and/or more quickly than using other types of disease burden information, such as AHI which may be obtained later after the patient has already experienced significant upper airway obstructions. In some examples, other information, such as muscle activity, may be sensed from at least one cricothyroid muscle 1022 (innervated by the eSL nerve 1010) using EMG.

In some examples, the second target tissue which is stimulated may include the iSL nerve 1008. For example, the second target tissue may comprise an afferent nerve fiber of the iSL nerve 1008 which is selectively stimulated. Stimulating the iSL nerve 1008, which includes afferent nerve fibers, may elicit reflex response opening of the upper airway. For example, eliciting the reflex opening of the upper airway may activate nerves, which cause contraction of a plurality of upper airway patency-related muscles for promoting upper airway patency. The plurality of muscles may include upper airway dilator muscles, such as (but not limited to) the genioglossus muscle, the hyoglossus muscle, and the geniohyoid muscle. In some such examples, selectively stimulating afferent nerve fiber(s) of the iSL nerve 1008 may invoke a reflex opening activity of an array (or substantially the entire array) of upper airway patency-related muscles, as previously described above. For example, by stimulating the single iSL nerve 1008 (or portion thereof), via its sensory pathway, the stimulation therapy may invoke a comprehensive response of a plurality (e.g., more than one) of the upper airway patency-related muscles as part of the reflex opening activity. In some examples, the reflex opening response is at least similar to intrinsic/physiological opening of the upper airway.

In some examples, the second target tissue which is stimulated may include other targets, such as a cricothyroid muscle 1022. As further described later in association with at least FIG. 17C, the second target tissue (e.g., 5130 in FIG. 17C) may comprise additional nerves/muscles such as (but not limited to) the hypoglossal nerve, the genioglossus muscle, the IHM-innervating nerve, the infrahyoid muscle(s), which sometimes may be referred to as upper airway patency-related motor nerves/muscles. In some examples, the second target tissue 5130 may comprise the phrenic nerve and/or the diaphragm muscle.

In some examples, multiple second target tissues may be stimulated, such as: stimulating the iSL nerve 1008 and the glossopharyngeal nerve; stimulating the iSL nerve and the IHM-innervating nerve or IHM(s); stimulating the iSL nerve 1008 and the hypoglossal nerve.

FIG. 12 illustrates example iSL nerves 1008R, 1008L located in the head-and-neck region. More particularly, FIG. 13 illustrates a front view of the head-and-neck region of the patient and the iSL nerves 1008R, 1008L, as previously described in connection with FIG. 11. The level of the vocal folds 1020 is shown in FIG. 13 as a dashed line. The common features and attributes are not repeated for ease of reference.

FIGS. 13, 14, 15, and 16 are diagrams schematically representing patient anatomy, may be used as target tissue by an example device and/or in an example method for sensing and/or stimulating an IHM-innervating nerve (and/or infrahyoid muscle (IHM), a hypoglossal nerve (and/or genioglossus muscle), and/or other target tissue.

As previously noted in connection with at least FIG. 1A, in some examples an upper airway patency-related motor nerve may comprise an IHM-innervating nerve in addition to, or instead of, a hypoglossal nerve.

In some examples, an IHM-innervating nerve may comprise a nerve or nerve branch which innervates (directly or indirectly) at least one infrahyoid muscle (IHM), which may sometimes be referred to as an infrahyoid strap muscle. In some examples, IHM-innervating nerves/nerve branches extend from (e.g., originates) from a nerve loop called the ansa cervicalis (AC) or the “AC loop nerve”, which stems from the cervical plexus, e.g., extending from cranial nerves C1-C3. Accordingly, in some examples, at least some IHM-innervating nerves may correspond to an ansa cervicalis (AC)-related nerve in the sense that such nerves/nerve branches (e.g., IHM-innervating nerves) do not form the AC loop nerve but extend from the AC loop nerve. At least because the AC loop nerve is the origin for some nerves which innervate muscles other than the infrahyoid muscles, some AC-related nerves do not comprise IHM-innervating nerves. Moreover, it will be understood that in some examples, stimulation applied to a portion (e.g., superior root) of the AC loop nerve (and/or to nerves from which the AC loop nerve originates) may activate IHM-innervating nerves/nerve branches, which extend from the AC loop nerve. However, implementing stimulation (e.g., to influence upper airway patency) occurring at more proximal locations, such as along the superior root of the AC loop nerve may be more complex because of the number/type of different nerves and number/type of different muscles innervated via a superior root of the AC loop nerve such that selective activation of a particular infrahyoid muscle (via stimulation along the superior root) may be quite challenging in some circumstances.

With this background in mind, FIG. 13 is a diagram 600 schematically representing patient anatomy and providing further details regarding example devices and/or example methods for stimulating an IHM-innervating nerve and/or hypoglossal nerve. As shown in FIG. 13, diagram 600 includes a side view schematically representing an AC-main nerve 615, in context with a hypoglossal nerve 605 and with cranial nerves C1, C2, C3. As shown in FIG. 13, portion 629A of the AC-main nerve 615 (e.g., a portion or trunk connecting to the AC loop nerve 619) extends anteriorly from a first cranial nerve C1 and a segment 617 running alongside (e.g., coextensive with) the hypoglossal nerve 605 for a length until the AC-main nerve 615 diverges from the hypoglossal nerve 605 to form a superior root 625 of the AC-main nerve 615, which forms part of the AC loop nerve 619. A portion of the hypoglossal nerve 605 extends distally to innervate the genioglossus muscle 604. As further shown in FIG. 13, the superior root 625 of the AC-main nerve 615 extends inferiorly (e.g., downward) until reaching near bottom portion 618 of the AC loop nerve 619, from which the AC loop nerve 619 extends superiorly (e.g., upward) to form an lesser root 627 (e.g., inferior root) which joins to the second and third cranial nerves, C2 and C3, respectively and via portions 629B, 629C.

As further shown in FIG. 13, several branches 631 extend off the AC loop nerve 619, including branch 632 which innervates the omohyoid muscle group 634, branch 642 which innervates the sternothyroid muscle group 644 and at least a portion (e.g., inferior portion) of the sternohyoid muscle group 654. Another branch 652, near bottom portion 618 of the AC loop nerve 619, innervates at least a portion (e.g., superior portion) of the sternohyoid muscle group 654. In some examples, the collective arrangement of the AC-main nerve 615 (including at least superior root 625 of the AC loop nerve 619) and its related branches (e.g., at least 632, 642, 652) when considered together, or any of those elements individually, may sometimes be referred to as an IHM-innervating nerve 616. It will be further understood that at least one such IHM-innervating nerve 616 is present on both sides (e.g., right and left) of the patient's body.

In some examples, stimulation of the superior root 625 of AC loop nerve 619 and/or at least some of the branches 631 extending from the AC loop nerve 619, may influence upper airway patency. However, in some examples, upper airway patency also may be increased and/or maintained by directly stimulating the above-identified muscle groups, such as the omohyoid, sternothyroid, and/or sternohyoid muscle groups. Accordingly, in some examples, such stimulation also may comprise stimulation of just a nerve portion(s), just muscle portion(s), a combination of nerve portion(s) and muscle portion(s), a neuromuscular junction of nerve portion(s) and muscle portion(s), and combinations thereof. Among other effects, in some examples stimulation of such nerves and/or muscles (and/or neuromuscular junctions, combinations, etc.) may act to bring the larynx inferiorly, which may increase upper airway patency.

Sensing may occur from and/or stimulation may be delivered to many different locations of an IHM-innervating nerve 616/nerve branches. Of these various potential sensing and/or stimulation locations, FIG. 13 generally illustrates three example sensing and/or stimulation locations A, B, and C. A sensing and/or stimulation element may be placed at all three of these locations or just some (e.g., one or two) of these example sensing and/or stimulation locations. At each location, a wide variety of types of sensing and/or stimulation elements (e.g., cuff electrode, axial array, paddle electrode, etc.) may be implanted depending on the particular delivery path, method, etc. For example, any one or a combination of the various example sensing and/or stimulation elements (and associated manner of access, delivery, etc.) described in association with at least FIGS. 1A-10C may be used to deliver such stimulation. In some such examples, a scale of the various stimulation elements, anchors, access tools, and/or other elements in some of the examples in FIGS. 1A-10C may be reduced to accommodate a generally smaller diameter of the IHM-innervating nerve/nerve branches 616 as compared to some other nerve portions, such as at least some portions of the hypoglossal nerve 605.

With further reference to FIG. 13, at each example sensing and/or stimulation A, B, C, a sensing and/or stimulation element may be delivered subcutaneously, intravascularly, etc. At each sensing and/or stimulation location, in some examples the stimulation element may comprise a microstimulator.

It will be understood that these example sensing and/or stimulation locations A, B, C are not limiting and that other portions along the IHM-innervating nerve 616/nerve branches may comprise suitable sensing and/or stimulation locations, depending on the particular objectives of the stimulation therapy, on the available access/delivery issues, etc.

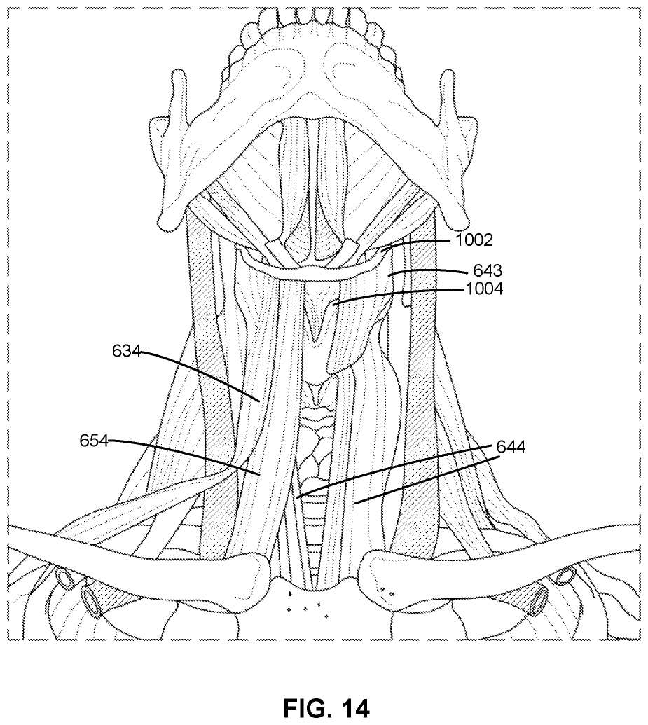

Among the different physiologic effects resulting from sensing and/or stimulation of the various portions of the IHM-innervating nerve 616/nerve branches (and/or innervated muscle portions, neuromuscular junctions, etc.), in some examples stimulation of nerve branches which cause contraction of the sternothyroid muscle 644 and/or the sternohyoid muscle 654 may cause the larynx to be pulled inferiorly, which in turn may increase and/or maintain upper airway patency in at least some patients. Such stimulation may be applied without stimulation of the hypoglossal nerve 605 or may be applied in coordination with stimulation of the hypoglossal nerve 605. More particularly, FIGS. 13-14 show example target tissue including or associated with an IHM-innervating nerve 616 and muscles 634, 644, 654 innervated thereby.

In some examples, different locations of the IHM-innervating nerve 616 may be target tissue for sensing and/or stimulating. That is, in some examples, the first target tissue and/or the second target tissue may comprise an IHM-innervating nerve 616 and/or an IHM 634, 644, 654. In some examples, the first target tissue and second target tissue comprise different portions of the IHM-innervating nerve 616 (e.g., target location A and C), while in some examples, the first target tissue and second target tissue may comprise a same portion of the IHM-innervating nerve 616 (e.g., target location C). In some examples, the first target tissue comprises the IHM-innervating nerve 616, while the second target tissue comprises the IHM-innervating nerve 616, at least one IHM 634, 644, 654, and/or the hypoglossal nerve 605. Non-limiting examples of the first target tissue and/or second target tissue locations may include the target locations labeled “A”, “B”, and “C”. In some examples, the first target tissue may comprise efferent nerve fibers (e.g., motor nerve fibers) of the IHM-innervating nerve 616, while in some examples, the first target tissue may comprise solely efferent nerve fibers of the IHM-innervating nerve 616. In some examples, the second target tissue may comprise efferent nerve fibers of the IHM-innervating nerve 616, while in some examples, the second target tissue may comprise solely efferent nerve fibers of the IHM-innervating nerve 616.

In some examples, sensing a first respiratory parameter from the IHM-innervating nerve 616 and/or the at least one IHM 634, 644, 654 comprises sensing neural activity that is phasic with respiration (and optionally, sleep disordered breathing events). For example, neural activity may be sensed from at least some portions of the IHM-innervating nerve 616. While FIGS. 17A-17B illustrate sensed neural activity for an iSL nerve, it will be understood that sensed neural activity from an IHM-innervating nerve 616 (and/or IHM(s)) may generally represented by FIGS. 17A-17B for illustrative simplicity. In some examples, the neural activity may be sensed from a portion of the IHM-innervating nerve 616 using ENG. As evident from the example of FIGS. 17A-17B, because the sensed neural activity is phasic with respiration, the sensed neural activity may be used to detect respiratory information including respiration parameters of respiratory phase information.

In some examples, as further illustrated by the example timing diagrams of FIGS. 17A-17B (e.g., for the iSL nerve), it will be understood that the sensed neural activity for the IHM-innervating nerve 616 also would exhibit an increase in amplitude and/or duty cycle as represented at 5025D, 5025E, 5028F and in response to an upper airway obstruction as represented at 5015D, 5015E, 5015F, respectively. According, the sensed neural activity of the IHM-innervating nerve 616 may be used to detect respiratory obstruction information in addition to the general respiratory information. In some other examples, the respiratory information may be sensed from an IHM 634, 644, 654 using EMG.

In some examples, the second target tissue which is stimulated may include the IHM-innervating nerve 616 and/or the at least one IHM 634, 644, 654. For example, the second target tissue may comprise at least one of the branches 631 extending from the AC loop nerve 619. The IHMs 634, 644, 654 may be innervated by the nerve branches 631, such that any of the nerve branches 631 may be considered example IHM-innervating nerve 616 or portions thereof. For example, the nerve branch 642 (at which target location C is located) of IHM-innervating nerve 616 extends distally from a superior root portion of the AC loop nerve 619 and innervates the sternothyroid muscle 644, which comprises one of the IHMs 634, 644, 654 which can be potentially stimulated. In some examples, the at least one IHM 634, 644, 654 comprises the sternothyroid muscle 644 and the inferior portion of the sternohyoid muscle 654, sometimes herein referred to as “sternohyoid muscle inferior”. In some examples, other IHMs are activated, such as the sternohyoid muscle 654 and/or the omohyoid muscle 634.

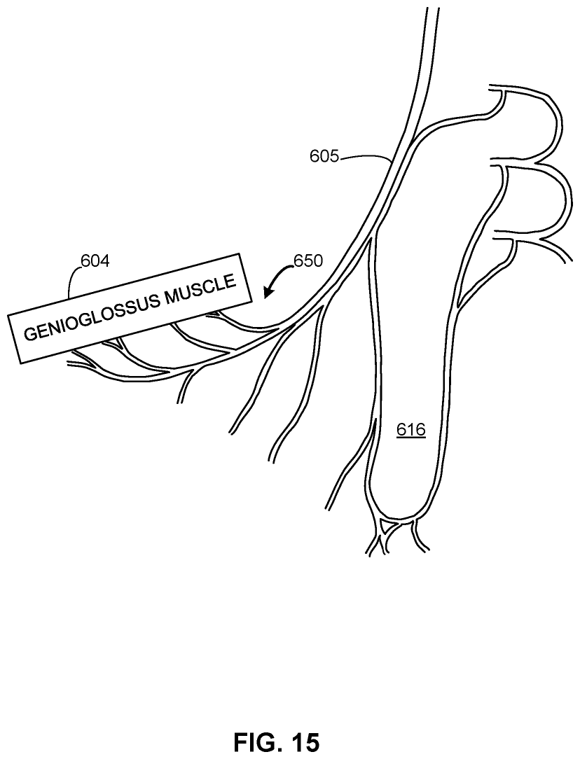

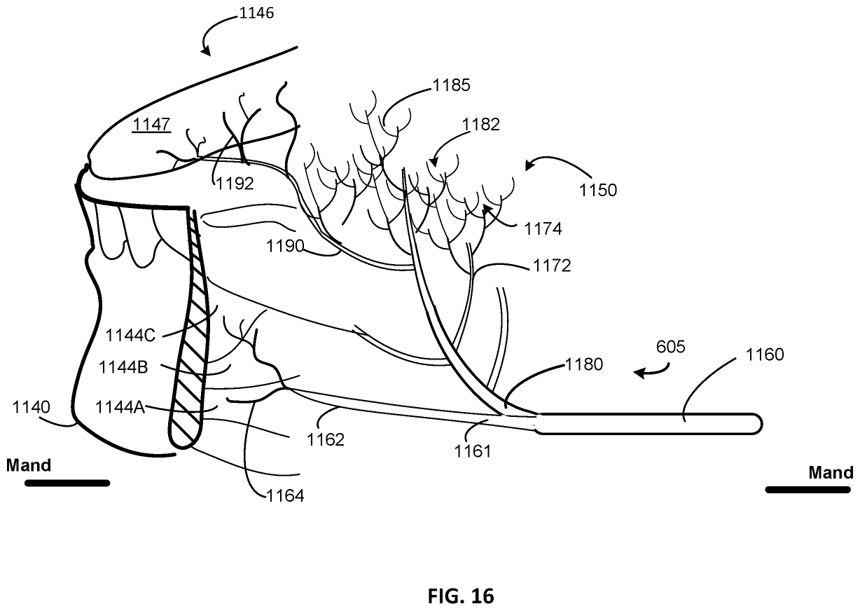

In some examples, the second target tissue which is stimulated may include the hypoglossal nerve 605, such as a distal portion of the hypoglossal nerve 605. In some such examples, the hypoglossal nerve 605 may be stimulated at a location (e.g., distally) and/or manner to activate at least (or solely) the protrusor muscles of the genioglossus muscle 604, as further described in connection with at least FIGS. 15-16.

In some examples, the second target tissue may include: (i) the hypoglossal nerve 605 and/or the IHM-innervating nerve 616, (ii) the hypoglossal nerve 605 and/or at least one IHM 634, 644, 654, or (iii) the hypoglossal nerve 605, the IHM-innervating nerve 616 and/or at least one IHM 634, 644, 654.

Among other effects, stimulation at the target location of the IHM-innervating nerve 616, such as but not limited to target location C, acts to bring the larynx inferiorly, which may increase upper airway patency. For example, stimulating the IHM-innervating nerve 616 or at least one muscle innervated thereby causes displacement of the thyroid cartilage (1004 of FIGS. 11-12) inferiorly, and thereby causes stiffening of a pharyngeal wall of the patient which increases and/or maintains patency of at least the oropharynx portion of the upper airway.

As described above, examples are not limited to sensing and stimulating the same target tissue. The different target tissues may include different portions of the IHM-innervating nerve 616, or different nerves or muscles (e.g., the IHM-innervating nerve 616). In some such examples, stimulating the second target tissue activates at least one upper airway patency-related muscle, such as at least one of the IHMs 634, 644, 654, the genioglossus muscle 604, or other muscles. For example, the first target tissue may comprise a first portion of the IHM-innervating nerve 616, and the second target tissue comprises a second portion of the IHM-innervating nerve 616 that is different from the first portion or the IHMs 634, 644, 654 (e.g., stimulating and sensing at target locations A and C). As another example, the first target tissue comprises the IHM-innervating nerve 616 and the second target tissue comprises the hypoglossal nerve 605 and/or the genioglossus muscle 604.

FIG. 14 illustrates example IHMs 634, 643, 644, 654 located in the neck region, at least a portion of which may be innervated by an IHM-innervating nerve. More particularly, FIG. 14 illustrates a front view of the head-and-neck region of the patient and the IHMs 634, 643, 644, 654 located in the head-and-neck region, including the omohyoid muscle 634 which overlies at least a portion of the sternohyoid muscle 654 and the sternothyroid muscle 644, as previously described in connection with FIG. 13. The thyrohyoid muscle 643 may not be innervated by the IHM-innervating nerve (616 in FIG. 13). Further illustrated by FIG. 14 is the thyroid cartilage 1004 and the hyoid bone 1002. In various examples, at least one of the IHMs 634, 643, 644, 654 may include the first and/or second target tissues, or may be activated in response to stimulating the second target tissue.

FIGS. 15-16 show example target tissue including or associated with a hypoglossal nerve 605 and muscles innervated thereby. As previously described and illustrated in connection with FIG. 13, at least a portion of the hypoglossal nerve 605 may extend proximate to the IHM-innervating nerve 616. The genioglossus muscle 604 is be innervated by the hypoglossal nerve 605. As shown, the hypoglossal nerve 605 includes distal branches 650 which may extend to the genioglossus muscle 604.

In some examples, different locations of the hypoglossal nerve 605 may be target tissue for sensing and/or stimulating. That is, in some examples, the first target tissue and/or the second target tissue may comprise the hypoglossal nerve 605 and/or the genioglossus muscle 604. In some examples, the first target tissue and second target tissue comprise the same or different portions of the hypoglossal nerve 605. In some examples, the first target tissue comprises the hypoglossal nerve 605 and the second target tissue comprises the hypoglossal nerve 605 and/or the genioglossus muscle 604.

In some examples, sensing the first respiratory parameter from the hypoglossal nerve 605 comprises sensing neural activity that is phasic with respiration (and optionally, sleep disordered breathing events). For example, neural activity may be sensed from the hypoglossal nerve 605, such as via ENG. It will be understood that, in some examples, the sensed neural signal may reveal neural activity occurring just prior to inspiration, which in some such examples may comprise a pre-inspiratory drive signal of the hypoglossal nerve. This pre-inspiratory drive signal causes protrusion of the tongue just prior to inspiration to ensure patency of the upper airway at the beginning of, and during at least the inspiratory phase. Similar to the illustrated example for the iSL nerve (e.g., FIGS. 17A, 17B) and IHM-innervating nerve, the sensed neural activity of the hypoglossal nerve may increase in amplitude and/or duty cycle in response to an upper airway obstruction.

The pre-inspiratory drive signal received from the central nervous system (CNS) is an effect received/caused as part of an overall reflex response opening of the upper airway as part of the general respiratory cycle, which is driven (at least in part) by activity of the phrenic nerve (and innervated diaphragm muscle which causes inspiration). Accordingly, the sensing of neural activity of the hypoglossal nerve comprises sensing of an efferent nerve fiber, by which one can determine impending inspiratory activity due to activation of the efferent/motor nerve from/as part of overall reflex opening response of upper airway.

In some examples, when an obstruction (e.g., flow limitation) of the upper airway occurs during a breath (e.g., intended inspiration), this obstructive event may be revealed in the sensed neural activity of the hypoglossal nerve prior to/during the next/subsequent inspiration in which a heightened reflex opening response occurs as effort by the CNS to overcome the obstruction to regain better/normal inspiration of fresh air.

Among other physiologic influences, in some examples, the sensed neural activity which corresponds to, and which reveals, upper airway obstruction may be associated with (and result from) mechanoreceptors located at or near the upper airway, as previously described.

In some examples, the sensing of the first respiratory parameter may comprise sensing respiratory tissue activity. For example, sensing of respiratory tissue activity may comprise sensing of respiratory-related muscles and/or other types of tissues from which respiratory information may be obtained. For instance, in some example, respiratory activity may be sensed from the genioglossus muscle 604 using electromyography (EMG). Other muscles may be sensed, in various examples and as previously and/or further described herein.

In a manner similar to the previously-described iSL nerve and via the later example illustrations (e.g., FIGS. 17A, 17B), because the sensed neural activity of the hypoglossal nerve and/or muscular activity (of the genioglossus muscle) is phasic with respiration, this sensed activity may be used to detect respiratory information including respiration parameters of respiratory phase information. This association is similar to the association illustrated by the timing diagrams of FIGS. 17A-17B for the iSL nerve, in which the sensed neural activity at 5023A, 5023B has an amplitude, duty cycle, duration associated with generally normal inspiratory phase 5012 of generally normal respiratory cycles 5011. While the timing diagrams of FIGS. 17A-17B illustrates an example shaped sensor signal, as may be appreciated, sensor signals sensed from different target tissue may exhibit different shapes and/or patterns, such as differences in amplitude and/or duration than illustrated by FIGS. 17A-17B.

Moreover, the sensed respiratory activity (e.g., sensed neural activity) associated with the hypoglossal nerve may increase in amplitude and/or duty cycle (as represented at 5025D, 5025E, 5025F) in response to an upper airway obstruction represented at 5015C, 5015D, 5015E, etc., respectively. Accordingly, the sensed activity associated with the hypoglossal nerve may be used to detect respiratory obstruction information in addition to the general respiratory information.

In some examples of the present disclosure, a second target tissue which is stimulated may include the hypoglossal nerve 605 and/or the genioglossus muscle 604, as shown in FIGS. 13, 15. Stimulating the second target tissue may activate at least one upper airway patency-related muscle, such as stimulating at least the nerve branch(s) (e.g., distal, medial branch(es) 650) of the hypoglossal nerve 605 which activates the genioglossus muscle 604.

As further illustrated in FIG. 15, for example, the second target tissue may comprise at least one of the branches 650, such as protrusor-related branches of the hypoglossal nerve 605, which when activated may cause protrusion of the tongue. Such protrusion, in turn, promotes (e.g., maintains and/or increases) upper airway patency. The genioglossus muscle 604 may be innervated by at least one of the nerve branches 650. In some such examples, stimulating the second target tissue of the hypoglossal nerve 605 causes the tongue muscle to stiffen and to protrude by activating at least the genioglossus muscle 604, and thereby promoting upper airway patency (e.g., dilating the upper airway).

As described above, examples are not limited to sensing and stimulating the same target. In some examples, stimulating the second target tissue activates at least one upper airway patency-related muscle, such as at least one of the IHMs 634, 644, 654, or other muscles, while the first target tissue (to be sensed) may comprise a nerve (e.g., hypoglossal nerve via ENG) or a muscle (e.g., genioglossus muscle via EMG) other than the particular nerve (e.g., IHM-innervating nerve) which innervates the muscle (e.g., IHM 634, 644, 654) being stimulated.

FIG. 16 is a side view schematically representing an example target tissue and locations for deploying sensing and/or stimulation components of a device. More particularly, FIG. 16 illustrates example target tissues associated with a hypoglossal nerve 605, such as tissues which may affect upper airway patency and hence which sometimes be referred to as upper airway patency-related tissue. As shown by FIG. 16, the hypoglossal nerve 605 comprises a medial branch 1160, which in turn comprises multiple distal branches (e.g., distal nerve portions) 1150. The medial branch 1160 includes proximal portions 1180, 1161 which may extend to distal branches 1150 or other distal segments of the proximal portions 1180, 1161.

In some examples, the sensing and/or stimulating may occur at the most distal segments of the nerve portion(s) and associated muscle portion(s), etc., of the hypoglossal nerve 605. For example, as shown in FIG. 16, one example distal terminal nerve portion 1185 of a group or region 1182 may be targeted for stimulation by stimulating the more proximal nerve portions (e.g., 1180, 1161). In some such examples, stimulation signals may be indirectly provided to the distal terminal nerve portions 1164, 1185, 1192 and/or less proximal nerve portions (e.g., 1172, 1190 which supports distal terminal nerve portion 1192).

As further shown in FIG. 16, the more distal terminal nerve portions (e.g., 1164) extending from nerve portion 1162 may innervate muscle portions 1144A, 1144B, 1144C which originate from an interior portion of the chin 1140. Moreover, the more distal terminal nerve portions (e.g., 1192) may innervate muscle portions 1147 closer to a top surface portion of the tongue 1146. Other groups 1182, 1174 of distal terminal nerve portions 1185 may innervate more proximal muscle portions of the tongue (genioglossus muscle), at least some of which are involved in causing protrusion of the tongue and hence which may sometimes be referred to as protrusor muscles.

In accordance with various examples of the present disclosure, sensing the first respiration parameter may comprise sensing neural activity, such as via ENG and/or EMG and using the sensed neural activity to determine the first respiration parameter. Example respiration parameters may include respiratory phase information and/or respiratory obstruction information. As described above, neural activity of various nerves may be in phase with respiration. In some examples, the neural activity has an onset that precedes the onset of inspiration and remains through the inspiratory phase of respiration. In some such examples, the neural activity sensed from the first target tissue may be used to detect inspiration, while stimulation is being applied at the same time or overlapping times to the second target tissue, and without the stimulation artifacts negatively impacting the sensing signal. In such examples, the sensing may be performed using techniques (e.g., ENG) in which the stimulation artifacts are not or minimally are present in the sensed signal. The respiratory obstruction information, as further described herein, may include a relative degree of upper airway obstruction.

Accordingly, in some examples, the first target tissue used to obtain respiratory information may include a nerve, such as the hypoglossal nerve, the iSL nerve, the IHM-innervating nerve, the phrenic nerve, and/or other nerves/muscles. In some such examples, such examples nerves may be easily accessible as a source for respiratory information and may allow for sensing and stimulating generally concurrently (e.g., during generally the same time frame), and without the stimulation artifacts impacting the sensed signal. In some examples, the same nerve may be used as the second target tissue to which stimulation may be applied. Using the same target tissue for sensing and stimulation may reduce surgical access requirements for placing electrode arrangements for stimulation and/or sensing. Moreover, in some areas of the body such as (but not limited to) the head-and-neck region, it may be challenging to implant some types of sensors and/or stimulation elements other than electrode arrangements. Similarly, the head-and-neck region (or other compact tissue areas) may pose challenges for obtaining sensing signals of sufficient quality and/or at a reasonable power demand.

FIGS. 17A-17J are diagrams illustrating example sensing protocols and/or stimulating protocols.

More specifically, FIGS. 17A and 17B are timing diagrams illustrating examples of a timing relationship between sensed neural activity and a respiration parameter. As described above, neural activity sensed from at least some example nerves may generally correspond to (e.g., be in phase with) respiration and may additionally be affected by upper airway obstruction. In some examples, respiration information may be determined from sensing activity of nerves (e.g., neural activity) indicative of respiration, including general respiratory information as well as respiratory obstruction information (e.g., upper airway obstruction).

For example, FIG. 17A is a timing diagram 5000 showing an example respiratory waveform 5010 and a sensed neural signal 5020. The sensed respiratory waveform 5010 is representative of respiratory activity sensed via pressure (e.g., in continuity with lung tissue) or via other modalities such as impedance, accelerometer, etc. to sense chest motion. Sensing via at least some examples of the present disclosure may be implemented instead of (or in addition to) the sensing modalities used to obtain respiratory waveform 5010. Accordingly, respiratory waveform 5010 provides a reference for comparison and by which further understanding may be gained regarding the various examples of sensed neural activity (or other sensed muscle activity or sensed tissue activity) of the present disclosure. The neural signal 5020, in the example, is sensed using ENG 5019.

Among other things, FIG. 17A provides an example respiratory waveform 5010, including an inspiratory phase 5012 having duration INSP, an active expiratory phase 5014 having duration EA, and an expiratory pause phase 5016 having duration EP. Together, these phases comprise an entire respiratory cycle 5011 having a duration (e.g., respiratory period) of R. This respiratory cycle 5011 is repeated, as represented in successive frames A, B, C, D, E, and so on. It will be understood that the respiratory cycles 5011 depicted in each frame A-C and D-E of FIG. 17A are respectively depicted as being identical, but in reality there may be variations in the respiratory cycle from breath-to-breath, and each patient may exhibit some variances in their respiratory waveform from other patients. For example, the respiratory cycles 5011 in frames C, D, E illustrate example waveforms responsive to an upper airway obstruction. As shown by frames C, D, E, in response to the obstruction, the duration (e.g., respiratory period) R increases, among other changes in the pattern of the waveform 5010.

While a neural signal may be sensed from any of the described nerve targets (and/or muscle targets) to obtain information representative of respiratory activity, for illustrative simplicity, signal 5020 in FIG. 17A depicts one example neural waveform sensed from the iSL nerve. The neural signal 5020 may comprise a respiratory signal cycle 5021, which includes first portion 5022, second portion 5024, and third portion 5026. In some examples, the first portion 5022 may generally correspond to inspiratory phase 5012 and may have a duration INSP. The second portion 5024 may generally correspond to active expiratory phase 5014 and may have a duration EA. The third portion 5026 may generally correspond to an expiratory pause phase 5026 and may have a duration EP. Accordingly, in some examples, the first, second, and third portions 5022, 5024, 5026 of the sensed neural activity may correspond to (e.g., be in phase with) the phases 5012, 5014, 5016 of the respiration waveform 5010. This sensed respiratory cycle 5021 is repeated in the successive frames A, B, C, D, E, and so on.

In general terms, the neural signal 5020 indicates activity of the iSL nerve during the inspiratory phase of each respiratory cycle and little (or no) neural activity of the iSL nerve thereafter, which corresponds to the expiratory phase. Accordingly, the sensed signal 5020 tracks neural activity generally representative of respiratory phase information.

As further shown by frames D, E, and F of the respiratory waveform 5010, when the patient experiences upper airway obstruction (e.g., 5015D, 5015E, 5015F), the first portion 5022A of the respiratory signal cycle 5021 of the iSL nerve exhibits an increase in duration and/or amplitude (as represented by dashed circle 5025D, 5025E, 5025F) of neural activity, among other changes in the pattern among the first, second, and third portions 5022A, 5024A, 5026A of the neural signal 5020 with such changes being indicative of the presence of an upper airway obstruction and a relative degree of obstruction.

FIG. 17B illustrates a timing diagram 5001 showing an example respiratory waveform 5010 and a sensed neural signal 5020, which may be an implementation of and/or include at least some of substantially the same features and/or attributes of the timing diagram 5000 of FIG. 17A, but with an additional example of a stimulation protocol 5030. The common features and attributes are not repeated for ease of reference.

More particularly, FIG. 17B further illustrates an example stimulation protocol 5030 for stimulating a second target tissue according to a respiratory parameter determined from the neural signal 5020. The stimulation protocol 5030 includes a stimulation pattern 5031 to stimulate the second target tissue comprising a stimulation cycle 5035 including a stimulation period 5032 and a non-stimulation period 5034, with the stimulation cycle 5035 being repeated through successive frames A, B, C, D, E and so on. As shown for the first stimulation cycle 5035, the stimulation pattern 5031 includes the stimulation period 5032 comprising an amplitude of N1 during the inspiratory phase 5012 and the subsequent non-stimulation period 5034 having an amplitude of zero during the expiratory phases 5014, 5016. In some examples, such as shown in FIG. 17B, this stimulation pattern 5031 may sometimes be referred to as being synchronous with the inspiratory phase (5012) of the patient's respiratory cycles (e.g., breathing pattern). In another aspect, this stimulation pattern 5031 may sometimes be referred to as being a closed loop stimulation pattern in that sensed respiratory information (e.g., sensed feedback) is used to time the stimulation period 5032 to coincide with the inspiratory phase (5012) of the patient's respiratory cycles (e.g., breathing pattern).

As previously described, in some examples, the neural signal 5020 is sensed from the nerve target (e.g., iSL nerve in one example) and may be associated with mechanoreceptors that are affected by respiration, such that the neural signal 5020 may be used to sense respiration parameters including respiratory phase information (e.g., inspiratory and expiratory phase information). As part of sensing respiratory information, the neural signal 5020 also may sense or provide respiratory obstruction information. In some examples, multiple respiration parameters may be sensed using the sensed neural activity. For example, using the neural signal 5020, a first respiration parameter comprising respiratory phase information may be sensed and a second respiration parameter comprising respiratory obstruction information may be sensed. In some examples, multiple neural signals may be sensed, which may be from the same or different target nerves, and used to determine the respiration parameters, such as further illustrated in connection with FIG. 17C.

In some examples, the first respiration parameter may be used to set stimulation of the second target tissue. For example, the stimulation may be set by: (i) setting timing of the stimulation according to the first respiration parameter, (ii) setting an amplitude of the stimulation according to the first respiration parameter, and/or (iii) selecting the second target tissue (from a set of targets) based on the first respiration parameter. In some examples, the stimulation may be timed with respect to the inspiratory phase, expiratory phase(s), duration, and/or other respiration information. In some examples, the amplitude of the stimulation may be set responsive to detecting a relative degree of upper airway obstruction using the respiratory obstruction information. In some examples, other information may be used in addition and/or alternatively to set the amplitude level, such as the frequency of obstructions, disease burden, etc. For example, in response to the sensed cycle 5021 being a length (e.g., relatively longer) and/or pattern associated with a particular relative degree of obstruction, the amplitude of the stimulation may be increased (or decreased in response to a lower relative obstruction degree than previously detected). As another example, the timing of the stimulation may be set in relation to respiration, detection of a sleep disordered breathing event, and/or other physiological signal(s).

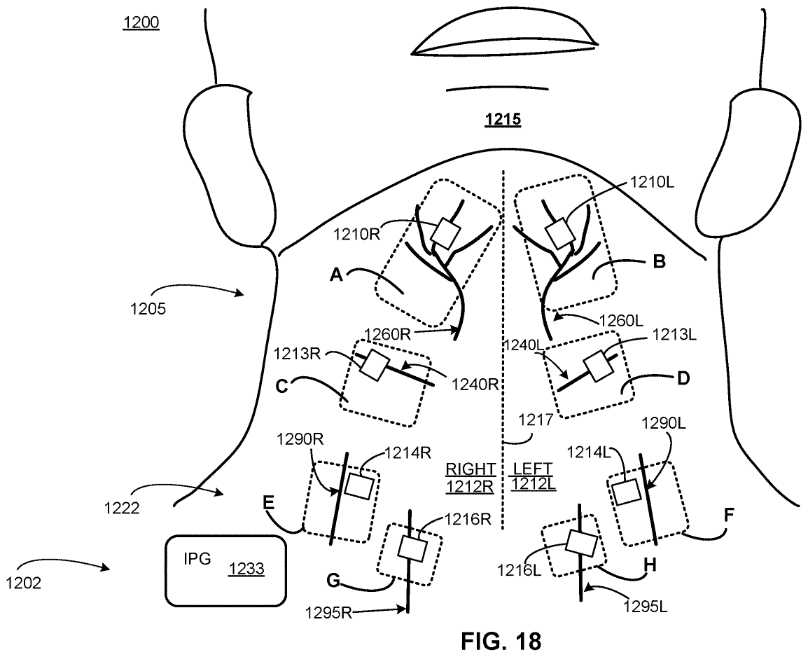

In some examples, based on the disease burden (e.g., AHI, ODI, etc.), relative degree of obstruction, and/or or other respiration information, the second target tissue may be selected from a set of target tissue. For example, a patient may have multiple electrode arrangements implanted, which are deployed proximate to each of the set of target tissue, such as further illustrated in connection with FIG. 18. The set of target tissue may comprise a set of respiratory tissue-related nerves, muscles innervated by the set of respiratory tissue-related nerves, and/or nerves whose stimulation elicit (via the CNS) respiratory responses (e.g., reflex opening response). In some examples, the set of respiratory tissue-related nerves include the hypoglossal nerve, the IHM-innervating nerve, the phrenic nerve, among other nerves as further described in association with at least FIG. 17C. The nerves whose stimulation elicit (via the CNS) respiratory responses (e.g., reflex opening response) may comprise the iSL nerve and/or afferent nerve fibers of the glossopharyngeal nerves associated with mechanoreceptors at or near the upper airway.

It will be understood that the stimulation protocol 5030 represented in FIG. 17B is merely just one example stimulation protocol and that other stimulation protocols may be implemented depending on type of target tissue (e.g., nerve or muscle), the particular role of the nerve and/or muscle in respiration generally and/or in upper airway patency, type of sleep disordered breathing, and/or other parameters.

FIG. 17C is a diagram 5100 illustrating an example arrangement of different target tissue(s) 5110 5130 for sensing and/or target tissues 5130 for stimulating.

In some examples, at least one of the target tissues 5110 may be used to sense a signal that generally corresponds to respiration to thereby provide information about a first respiration parameter 5105. The signal may be sensed from one of the target tissues 5110, on one or both lateral sides of the patient, and/or using a combination of the target tissues 5110. In some examples, one of the target tissues 5110 may be the first target tissue used to sense a first neural signal (and/or muscle signal), and a second target tissue nerve may be used if the first neural signal (and/or muscle signal) cannot be used (e.g., is no longer sensed, is noisy or other issues).

In some examples, the target tissues 5110 to be sensed may comprise an infrahyoid muscle (IHM)-innervating nerve 5112A, an IHM 5113A, a hypoglossal (HG) nerve 5114A, a genioglossus muscle 5115A, an internal superior laryngeal (iSL) nerve 5116A, a glossopharyngeal nerve 5117A, a phrenic nerve 5118A, a diaphragm muscle 5119A, and/or other nerves/muscles 5120A.

Meanwhile, the target tissues 5130 to be stimulated may comprise an IHM-innervating nerve 5112B, an IHM 5113B, an HG nerve 5114B, a genioglossus muscle 5115B, an iSL nerve 5116B, a glossopharyngeal nerve 5117B, a phrenic nerve 5118B, a diaphragm muscle 5119B, and/or other nerves/muscles 5120B.

In some examples, at least one of the target tissues 5130 may be stimulated. In some such examples, the stimulation is based on the sensed first respiration parameter 5105 and/or sensed other physiologic parameter. As previously described, any one of the respective target tissues 5110 may additionally serve as the target tissue(s) 5130 to be stimulated, in some examples. In some examples, multiple (e.g., at least two) of the target tissues 5130 may be stimulated. The stimulation of the multiple target tissues 5130 may occur simultaneously and/or sequentially. In some examples, such as those described above, the stimulation and sensing of the target tissues 5130 may be timed, such that sensing occurs at different times than stimulation. For example, a first target tissue may be sensed for a first plurality of sensing cycles to determine the first respiration parameter 5105 and then second target tissue may stimulation for a second plurality of stimulation cycles.

The timing, duration, amplitude, and/or selection of the target tissues 5130 to be stimulated may be set based on the signal (e.g., neural or muscle) sensed from at least one of the target tissues 5110. As a specific, and non-limiting example, the iSL nerve 5116A may be used to sense the first respiratory parameter and the iSL nerve 5116B (same or different portion) may be stimulated to elicit (via the CNS) the previously described reflex opening response that activates at least some of the target tissues 5130, such as (but not limited to) the HG nerve 5114B, the IHM-innervating nerve 5112B, which in turn causes activation (e.g., contraction) of their innervated muscles (e.g., upper airway dilators, such as the IHM 5113B and genioglossus muscle 5115B).

In some examples, a neural signal sensed from the iSL nerve 5116A may indicate an upper airway obstruction is occurring and/or continues after stimulating the iSL nerve 5116A. For example, for some patients, stimulating the iSL nerve 5116B to cause the reflex opening response may not be effective in increasing upper airway patency to a sufficient degree to ameliorate obstructive sleep apnea. In response, additional target tissue 5130 may be stimulated. For example, both the iSL nerve 5116B and other tissue, such as the IHM-innervating nerve 5112B or IHM 5113B, may be stimulated. In some such examples, other information indicative of a disease burden (e.g., AHI) may additionally or alternatively indicate to stimulate the additional target tissue(s) 5130.

It will be understood that some nerves/muscles may be considered to be upper airway patency-related tissue (e.g., nerves/muscles) in that direct sensing and/or direct stimulation of such nerves/muscles may have a direct effect on upper airway patency. For instance, stimulation of the HG nerve 5114B may cause protrusion of the tongue (via activation of the genioglossus muscle), which directly maintains and/or increases patency of the upper airway. Similarly, stimulation of the IHM-innervating nerve 5112B may cause (via activation of the sternothyroid muscle and/or other infrahyoid strap muscles), which may directly maintain and/or increase patency of the upper airway.

In some examples, stimulation of some target tissues 5130, such as the iSL nerve 5116B and/or afferent nerve fibers/branch of the glossopharyngeal nerve 5117B, may have an indirect effect, such as eliciting (via the CNS) a reflex opening response, which activates at least multiple upper airway dilator nerves/muscles. Such nerves are sometimes herein referred to as upper airway reflex-related sensory nerves. For instance, stimulation of afferent nerve fibers of the iSL nerve (and/or afferent nerve fibers/branch of the glossopharyngeal nerve) associated with mechanoreceptors in/near the upper airway may elicit (via the CNS) a reflex opening response to maintain and/or increase upper airway patency.

Meanwhile, in some examples, some target tissues may be used to affect respiration in other ways and/or more generally. For instance, an immediate effect of stimulation of the phrenic nerve 5118A includes activation of the diaphragm muscle 5119A, whose contraction induces a negative pressure within the lungs, thereby resulting in inspiration of air (passing through the upper airway) and other structures.

It will be understood that some example devices and/or some example methods may engage the phrenic nerve solely for stimulation to treat various types of apnea (e.g., central, mixed, other). However, some example devices and/or some example methods may engage the phrenic nerve solely for sensing or may engage the phrenic nerve for both sensing and stimulation.

With this in mind, FIG. 17D schematically represents an example arrangement 6200 including example sensing patterns for the phrenic nerve and/or example stimulation protocols.

For example, like FIGS. 17A-17B, FIG. 17D includes an example respiratory waveform 5010 obtained via sensing respiratory tissues (e.g., tissues in continuity with the lungs) and/or motion (e.g., chest) indicative of respiratory activity.

As further shown in FIG. 17D, an example respiratory waveform 6210 of respiratory activity obtained via sensing a phrenic nerve (e.g., 5118A in FIG. 17C) in which each instance 6212 of phrenic nerve activity generally coincides with the inspiratory phase (e.g., 5012) of a respiratory cycle. In some examples, the sensing is performed via ENG. Each instance 6212 of phrenic nerve activity includes an onset 6214 at which the phrenic activity begins and an offset 6216 at which the phrenic activity ceases, following by little to no neural activity as represented by segment 6125 during expiration takes place. Among other things transpiring during inspiration (or just before inspiration), the activation of the phrenic nerve causes contraction of the diaphragm to induce a negative pressure in the lungs, resulting in inspiration as air enters the lungs from the upper airway and external environment.

As shown in FIG. 17D, an amplitude of phrenic nerve activity generally increases from the onset 6214 to the offset 6216 at which time the amplitude abruptly decreases to zero or near zero. During a second phase (e.g., expiratory active phase and expiratory pause) of the respiratory cycle, the phrenic activity remains at or near zero, until the next onset 6214 of an inspiratory phase of a next respiratory cycle. Accordingly, the presence of phrenic nerve activity is directly indicative of inspiratory activity.

It will be understood that the phrenic activity waveform 6210 in FIG. 17D is just one example and that some small variations in amplitude (and/or duty cycle, timing, etc.) of the sensed phrenic nerve activity may exist when sensing at different portions of the phrenic nerve, among different patients, etc.

With this in mind, FIG. 17F further illustrates example respiratory activity waveforms 6330, 6350 obtained via sensing activity of a first phrenic nerve site and sensing activity of a first diaphragm muscle site, respectively. In some examples, the phrenic nerve activity of waveform 6330 is sensed via ENG while in some examples, the diaphragm muscle activity represented by waveform 6350 is sensed via EMG.

While some example devices and/or example methods may sense both activity of the phrenic nerve(s) and activity of the diaphragm muscle(s), some example devices and/or example methods may sense phrenic nerve activity without sensing diaphragm muscle activity and some example devices and/or example methods may sense diaphragm muscle activity (i.e., without sensing phrenic nerve activity).

In a manner consistent with the waveform 6210 in FIG. 17D of sensed phrenic activity, each instance 6332 of phrenic nerve activity in waveform 6330 of FIG. 17E generally coincides with the inspiratory phase (e.g., 5012 in FIG. 17D) of a respiratory cycle. Each instance 6332 of phrenic nerve activity as sensed at first phrenic site 6331 includes an onset 6334 at which the phrenic activity begins and an offset 6336 at which the phrenic activity ceases. Meanwhile, a non-active (e.g., rest or dormant) period 6337 extends between the instances 6332, 6332 of phrenic nerve activity. The peak amplitude of the phrenic nerve activity is represented as AMP 1, while a duration D1 of each instance of phrenic nerve activity generally corresponds to a duration of an inspiratory phase (e.g., 5012 in FIG. 17E).

In a manner consistent with the waveform 6330 of sensed phrenic activity, each instance 6352 of diaphragm muscle activity in waveform 6350 of the example arrangement 6300 of FIG. 17E generally coincides with each instance of 6332 of phrenic nerve activity (6330) and generally coincides with the inspiratory phase (e.g., 5012 in FIG. 17D) of a respiratory cycle. Each instance 6352 of diaphragm muscle activity as sensed at first diaphragm site 6351 includes an onset 6354 at which the diaphragm activity begins and an offset 6356 at which the diaphragm activity ceases. Meanwhile, a non-active period 6357 extends between the instances 6352 of diaphragm muscle activity. The peak amplitude of the diaphragm nerve activity is represented as AMP 2, while the duration D2 of each instance of diaphragm muscle activity generally corresponds to a duration of an inspiratory phase (e.g., 5012 in FIG. 17E).

With further reference to FIG. 17D, one example arrangement comprises delivering stimulation to a stimulation target (STIM TARGET 6249). In some examples, a timing of the stimulation may be based on at least one parameter of the sensed phrenic activity (and/or diaphragm muscle activity per FIG. 17E), which comprises a respiration parameter. In some examples, the delivery of the stimulation signal (e.g., stimulation protocol 6220 of FIG. 17D) is timed to generally coincide with the start, duration, and/or end of the sensed phrenic activity (e.g., waveform 6210 in FIG. 17D). In the particular example shown in FIG. 17D, an onset (B) of each instance of stimulation 6243 begins just prior to a start of an inspiratory phase (e.g., prior to onset 6214 of the sensed phrenic nerve activity). More generally speaking, the stimulation protocol 6220 in FIG. 17D comprises a series of stimulation cycles 6255, with each cycle 6255 including a stimulation period 6243 and a non-stimulation period 6245.

In some examples, an amplitude setting (and/or other parameters such as timing, duty cycle, etc.) of the stimulation signal may be based, at least on part, on the amplitude (and/or other parameters) of the sensed activity of the phrenic nerve and/or diaphragm muscle.

In some examples, the stimulation target 6249 (e.g., second target tissue 5130) represented in FIG. 17D may comprise an upper airway patency-related tissue such as (but not limited to) a hypoglossal nerve (and/or genioglossus muscle). In some such examples, the sensed phrenic neural activity (or diaphragmatic activity) provides highly accurate respiratory information in view of its high fidelity relative to respiration, which in turn may increase the effectiveness of delivery of the stimulation signal to treat sleep disordered breathing (e.g., obstructive sleep apnea).

In some examples, the target tissue (STIM TARGET 6249) may comprise tissues in addition to, or other than, the hypoglossal nerve and/or genioglossus muscle. For example, for some types of patients which may not respond sufficiently to stimulation of the hypoglossal nerve and/or genioglossus muscle, applying stimulation to an infrahyoid muscle (IHM)-innervating nerve (and/or innervated muscles such as (but not limited to) the sternothyroid muscle) may achieve efficacious stimulation therapy.

In some examples, the target tissue (STIM TARGET 6249) may comprise tissues in addition to, or other than, the hypoglossal nerve, genioglossus muscle, IHM-innervating nerve, and/or IHMs. For example, some types of patients may respond better to stimulation of one or more of the other second target tissues 5130 of FIG. 17C, whether standing alone or in combination with other second target tissues 5130.

Among other second target tissues, in some examples the stimulation target (STIM TARGET 6249) may comprise the phrenic nerve, which is the same nerve from which sensed neural activity (e.g., waveform 6210) is obtained. Among other factors affecting a choice to stimulate the phrenic nerve, such an example arrangement may enable an efficient and convenient implant procedure in that the same electrode arrangement (or different electrode arrangements in close proximity) may be used for sensing and stimulation.

With this in mind, FIGS. 17F-17H schematically represent example sensing protocols, example stimulation protocols, etc. for at least some target tissues 5110, 5130 (FIG. 17C), respectively. In some examples, such sensing protocols are applicable to the phrenic nerve (and/or diaphragm muscle).

In some examples, a sensing element (e.g., 110 in FIG. 1A) may comprise a particular type of sensing modality, and/or may be located in sufficiently close proximity to a stimulation element (e.g., 120 in FIG. 1B), such that performing sensing and stimulation simultaneously (or in very close temporal proximity) may be problematic at least because the magnitude and effect of applying stimulation significantly hinders reliably obtaining an accurate, useful sensing signal. At least some of the examples of FIGS. 2A-6 provide arrangements to implement sensing and stimulation in such situations.

FIGS. 17F-17H schematically represent further example sensing and stimulation protocols to coordinate timing of sensing and stimulation in such situations.

As shown in FIG. 17F, sensing activity periods 6504 may be alternated with stimulation application periods 6506 with a buffer period 6505 (having duration B1, B2, B3, and so on) therebetween. In general terms, a duration of the buffer periods 6505 is selected to ensure that the physiologic environment has settled sufficiently following a stimulation period 6506 to then permit effective and reliable sensing. The sensing may include sensing of a particular target tissue generally and/or for a particular parameter, such as first respiration parameter 5105 (FIG. 17C) and/or other physiologic parameter 5106 (FIG. 17C) which may or may not relate to respiration.

In some examples, the duration (B1, B2, B3) of buffer period 6505 between sensing and stimulation may be based on a distance between the sensing element (e.g., 110 in FIG. 1A) and the stimulation element (e.g., 120 in FIG. 1A), location and relationship of the respective sensed and stimulated target tissues, intervening tissues (bone, muscle, etc.), intrinsic timing/behavior of each respective nerve, muscle, and/or other physiologic factors.

It will be understood that in some examples, each stimulation application period 6506 may comprise multiple spaced apart instances 6722 of stimulation, with each instance 6722 of stimulation comprising a segment of continuous pulsed stimulation (e.g., a train of stimulation pulses according to a duty cycle), such as (but not limited to) an example series 6720A of stimulation periods 6722 as shown in FIG. 17H.

It will be understood that in some examples, each sensing activity period 6504 may comprise multiple spaced apart instances 6562 of sensing activity, such as (but not limited to) an example series 6560A of sensing activity periods 6562 as shown in FIG. 17G.

In some examples, saving power, managing overall stimulation volume, etc. may provide additional or alternative reasons to implement an example sensing and stimulation protocol like example protocol 6500 or one of the example protocols 6550, 6700 further described below in association with at least FIGS. 17G, 17H.

In some example, the sensing activity periods 6504 correspond to sensing at least one of the target tissues 5110 in FIG. 17C and the stimulation application periods correspond to stimulating at least one of the target tissues 5130 in FIG. 17C. To the extent that the activity of more than one different target tissue is being sensed, in some examples such sensing may be performed simultaneously during each sensing activity period 6504. However, in some examples, the sensing of different target tissues (5110) may be alternated in various manners such that one sensing activity period 6504 may sense a first sensing target tissue while a subsequent sensing activity period 6504 may sense a different second target tissue.

FIG. 17G schematically represents one example sensing protocol 6550 which may be implemented via example methods (and/or example devices) including (but not limited to) the example protocol 6500 of FIG. 17F and/or in association with various examples throughout the present disclosure. Among other examples, the sensing protocol 6550 may be implemented as a standalone sensing protocol, in conjunction with the example stimulation protocol 6700 of FIG. 17H, or in conjunction with various example stimulation protocols (e.g., methods and/or devices) of the present disclosure.

As shown in FIG. 17G, the sensing protocol 6550 comprises a plurality of spaced apart sensing activity periods 6560A, 6560B, 6560C, and so on, with each sensing activity period 6560A, 6560B, 6560C including at least one instance 6562 of sensing activity (SA). In examples in which a sensing activity period 6560A, 6560B, 6560C may comprise multiple instances 6562 of sensing activity (SA), non-sensing activity segments 6564 are interposed between successive instances 6562 of sensing activity (SA). Each instance 6562 of sensing activity comprises a duration G1 and each non-sensing segment 6564 comprises a duration G2.

In some examples, the sensing activity (SA) may comprise sensing respiratory activity (SA) such as sensing neural activity, muscular activity, and/or other types of activity indicative of respiration. Accordingly, in some such examples, the instances 6562 of sensed activity (SA) regarding respiration may comprise an inspiratory phase of respiration and/or other respiration information. In these examples, the non-sensing activity segment 6564 may comprise or correspond to an expiratory phase of respiration in which little or no respiratory activity can be sensed due to the temporary inactivity of the particular target nerve (e.g., hypoglossal nerve, phrenic nerve, etc.) and/or target muscle. Stated differently, the presence and duration of the instances 6562 and non-sensing activity segment 6564 depend on the particular type of target tissue (e.g., 5110 in FIG. 17C) being sensed. For examples, in some arrangements, the non-sensing activity segment 6564 may correspond to physiologic phenomenon other than expiration and may have consistent or variable duration from instance to instance.

In some examples, each series 6560A, 6560B, 6560C in FIG. 17G may comprise a greater number or fewer number of instances 6562 of sensing activity (SA).

As further shown in FIG. 17G, in some examples the protocol 6550 comprises “no sensing activity” periods 6570 interposed between the respective series 6560A, 6560B, 6560C of sensing activity, with each “no sensing activity” period having a duration NS1, NS2, and so no. In some examples, the duration (NS1, NS2) of different “no sensing activity” periods 6570 may be uniform.

In some examples, stimulation may be performed during the “no sensing activity” periods 6570 with duration NS1, NS2 being sufficient to enable performing stimulation without compromising an integrity (e.g., accuracy, stability) of the sensing activity. In some such examples, the duration NS1, NS2 is sufficient to encompass at least stimulation and a buffer (e.g., 6505) of no stimulation after the stimulation.

FIG. 17H schematically represents one example stimulation protocol 6700 which may be implemented via example methods (and/or example devices) including (but not limited to) the example protocol 6500 of FIG. 17F and/or in association with various examples throughout the present disclosure. Among various examples, in some examples the stimulation protocol 6700 may be implemented as a standalone stimulation protocol, in conjunction with the example sensing protocol 6550 of FIG. 17G, or in conjunction with various example stimulation protocols (e.g., methods and/or devices) of the present disclosure.

As shown in FIG. 17H, the stimulation protocol 6700 comprises a plurality of spaced apart stimulation application periods 6720A, 6720B, and so on, with each stimulation application period 6720A, 6720B including at least one instance 6722 of stimulation application. In examples in which a stimulation application period 6720A, 6720B may comprise multiple instances 6722 of stimulation application (ST), non-stimulation segments 6724 are interposed between successive instances 6722 of stimulation application (ST). Each instance 6722 of stimulation comprises a duration ST1 and each non-stimulation segment 6724 comprises a duration NST1.

In some examples, the stimulation application (ST) may comprise stimulation therapy regarding respiration. Accordingly, in some such examples, the instances 6722 of stimulation application (ST) may be directed to target tissues (e.g., 5110 in FIG. 17C) relating to respiration, and in some of these examples, the target tissues may comprise upper airway patency-related tissue. In some examples, stimulation of the target tissues may be used to treat obstructive sleep apnea, while in some examples, stimulation of the target tissues may be used to treat central sleep apnea. In some examples, stimulation of the target tissues may be used to treat multiple type apnea including aspects of both obstructive sleep apnea and central sleep apnea.

In some examples, a timing of the instances 6722 of stimulation application (ST) may be based, at least in part, on sensed activity (SA). However, in some such examples, the sensed activity (SA) may be performed separately and during a time frame other than the time frame during which stimulation application occurs such that the stimulation is not considered to be closed-loop stimulation in at least some respects (e.g., synchronized). Instead, the timing of the stimulation may be considered open loop stimulation for not being synchronized to on-going sensed activity.

Accordingly, in some examples, the timing of instances 6722 of stimulation application may be based on various parameters (e.g., phases, fiducials, aspects, etc. of previously sensed respiratory activity such as (but not limited) an inspiratory phase, an expiratory phase, onsets/offsets of those phases, midpoint crossing points of those phases, etc. However, as noted below, such stimulation application may be performed in an open loop manner, in some examples as further described below.

In some examples, various parameters (e.g., timing, amplitude, duty cycle) of the instances 6562 (FIG. 17G) of sensed activity (SA) may be used as a reference to, at least partially determine, various parameters (e.g., timing, amplitude, duty cycle) of the instances 6722 (FIG. 17H) of stimulation application. For instance, sensed activity corresponding to regular respiration (e.g., in which few or no upper airway obstructions occur) will result in first respiratory waveform having a particular shape, duration, etc., and then a corresponding stimulation application having a particular shape, duration, etc. However, sensed activity corresponding to many and/or significant obstructions in the upper airway will result in second respiratory waveform having a particular shape, duration, etc., (different from the second first respiratory waveform) and then a corresponding stimulation application having a particular shape, duration, etc. (e.g., increased amplitude, duration, and/or duty cycle) aimed at overcoming the obstructive behavior in order to restore regular respiration.

In general terms, because at least some examples seek to avoid performing sensing in close temporal proximity to stimulation, some example stimulation protocols may use historical sensed activity (SA) information for timing the instances 6722 of stimulation application, which still retaining an open loop behavior because the stimulation timing does not coincide with (e.g., is not synchronized to and/or not triggered by) a parameter (e.g., inspiratory phase) of a regular on-going sensing signal.

In some examples, each series 6720A, 6720B, etc. in FIG. 17H may comprise a greater number or fewer number of instances 6562 of stimulation application (ST).

As further shown in FIG. 17H, in some examples the protocol 6700 comprises pause periods (e.g., “no stimulation application” periods) 6730 interposed between the respective series 6720A, 6720B, etc., of stimulation application, with each pause period having a duration P1, P2, and so on. In some examples, the duration (P1, P2) of different pause periods 6730 may be uniform.

In some examples, sensing may be performed during the pause (“no stimulation application”) periods 6730 with duration P1, P2 being sufficient to enable performing stimulation without compromising an integrity (e.g., accuracy, stability) of any sensing activity which may be performed during the pause periods 6730. In some such examples, the duration P1, P2 is sufficient to encompass at least performing sensing and a buffer (e.g., 6505) prior to the sensing in which no stimulation is performed.

Accordingly, in some examples, the sensing protocol 6550 and stimulation protocol 6700 may be implemented in a complementary manner in which a series 6560A of instances 6562 of sensing activity (SA) is performed during a pause (“no stimulation period”) period 6730 of stimulation protocol 6700, with a sufficient buffer (e.g., 6505 in FIG. 17F) to ensure accuracy and integrity of the sensing signal. Stated differently, the stimulation protocol 6700 and sensing protocol 6550 may be implemented in a complementary manner in which a series 6720A of instances 6722 of stimulation application (ST) is performed during a “no sensing” period 6570 of sensing protocol 6550, with a sufficient buffer (e.g., 6505 in FIG. 17F) to ensure accuracy and integrity of the sensing signal.

More generally speaking, in some examples the complementary implementation of the sensing protocol 6550 and stimulation protocol 6700 may be sometimes be viewed as (or referred to as) an example method in which sensing is performed for a selectable predetermined number of units (e.g., breaths, seconds, minutes, etc.) to establish reliable sensed information (e.g., respiratory information) on which stimulation may then be applied (without concurrent sensing) for a selectable predetermined number of units (e.g., breaths, seconds, minutes, etc.), followed by a subsequent sensing-only period, subsequent stimulation-only period, and so on. In some examples, a value or quantity of the selectable predetermined number of units during which sensing is performed comprises the same value or quantity of the selectable predetermined number of units during which stimulation is applied. However, in some examples, a value or quantity of the selectable predetermined number of units during which sensing is performed comprises a different value or quantity of the selectable predetermined number of units during which stimulation is applied. In some example methods and/or devices, the selectable predetermined number may be varied throughout a treatment period (e.g., nightly sleep period) to facilitate a more robust for some situations in which the underlying conditions affecting sleep disordered breathing may be variable within/during a treatment period.

In some examples, the sensing protocol 6550 may be generally the same for at least some different target tissues (e.g., 5110 in FIG. 17C) and in some examples, the sensing protocol 6550 may be different for at least some different target tissues (e.g., 5110 in FIG. 17C).

In some examples, the stimulation protocol 6700 may be generally the same for at least some different target tissues (e.g., 5130 in FIG. 17C) and in some examples, the stimulation protocol 6700 may be different for at least some different target tissues (e.g., 5130 in FIG. 17C).

FIG. 17I schematically represents an example arrangement 6800 including an example implant access-incision 6810 as part of example methods and/or example devices for delivering sensing elements and/or stimulation elements for use in methods/devices of treatment. In some examples, the example arrangement 6800 may comprise an example implementation of, and/or at least some of substantially the same features and attributes of, at least some of the example methods and/or example devices as described throughout examples of the present disclosure.

As shown in FIG. 17I, in order to access at least some of the target tissues (e.g., 5110, 5130 in FIG. 17C) and/or other target tissues, one example arrangement 6800 includes an example method (and/or example devices) forming and/or using an implant-access incision 6810 in a head-and-neck region 6805 of the patient. In some examples, the implant-access incision 6810 may comprise a location about 3 to about 5 centimeters (as represented via arrow IA 1) superior to a clavicle 6815. In some examples, the implant-access incision 6810 is sized, shaped, oriented and/or located to provide access to a portion of a phrenic nerve 5118A/5118B (FIG. 17C) for sensing and/or stimulating the phrenic nerve 5118A/5118B while simultaneously providing access to an infrahyoid muscle (IHM)-innervating nerve 642 (e.g., nerve portion innervating the sternothyroid). In some examples, the same implant-access incision 6810 also may be used to access an IHM, such as the sternothyroid muscle 644 (FIG. 13), as just one example.

Among other aspects, the implant-access incision 6810 may a single implant-access incision through which all of the implantable elements of an example device and/or for an example method may be delivered into a chronically implanted position (e.g., subcutaneously) within the patient's body, such as head-and-neck region in some examples. For instance, both a sensing element 110 and a stimulation element 120 may be delivered and secured within the body via the single implant-access incision. In some such examples, it will be understood that the sensing element and/or stimulation element may comprise power elements, control elements, communication elements, or combinations thereof such that the implanted system may include all components suitable for operation independently from an external devices for at least certain periods of time. In some such examples, some or all of these implanted components when viewed collectively may be comprise a microstimulator or may comprise an IPG sized/shaped for implantation in a head-and-neck region.

Among other aspects, the implant-access incision 6810 enables quick, convenient, and effective access to a portion of the phrenic nerve 5118A/5118B which is remote from (e.g., having an inferior orientation and spaced apart from) to the more complex nesting of nerves, muscles, tissues, bones, ligaments, etc. in more superior anatomical locations at which the phrenic nerve also may be accessed such as proximate the mandible (and/or similar locations) at which other nerves (e.g., hypoglossal nerve) are often accessed for implantation of stimulation elements. Similarly, the implant-access incision 6810 enables quick, convenient, and effective access to select IHM-innervating nerve (e.g., 642) which is closer to an innervated muscle (e.g., sternothyroid muscle) which may be of more particular therapeutic interest, and which is remote from (e.g., inferior) to the more complex nesting of nerves, muscles, tissues, bones, ligaments, etc. in more superior anatomical locations at which the ansa cervicalis nerve loop 619 may be generally accessed and at which other nerves (e.g., hypoglossal nerve) also may accessed for implantation of stimulation elements.

Moreover, the example implant-access incision 6810 also may offer quick, convenient access to non-nerve anatomical structures in a less crowded environment and/or which are easier to visualize, which may aid in locating desired nerves, muscles as well as aid in locating/employing structures to which the sensing element(s), stimulation element(s), and/or other elements may be anchored.

Among other recognizable anatomical landmarks/structures, the implant-access incision 6810 may enable visualizing the internal jugular vein (IJV) 6820 and the position or orientation of the phrenic nerve 5118A/5118B being dorsal to the IJV 6820 and the IHM-innervating nerve (e.g., branch 642 innervating the sternothyroid muscle) being ventral (e.g., anterior) to the IJV 6820.

It will be further understood that at least some of the other target tissues 5110, 5130 (FIG. 17C) may be additionally or alternatively accessed via the implant-access incision 6810. Moreover, implant-access incision 6810 may enable access to target tissues other than those enumerated in association with at least FIG. 17C.

With this in mind, in some examples the implant-access incision 6810 may be used to implant an accelerometer 6920 (and/or other sensing element) as described below in association with FIG. 17J.

FIG. 17J schematically represents chronic implantation 6900 of an accelerometer 6920 (e.g., three-axis accelerometer) at or near the hypopharynx 6910, such as along or near walls 6912 of the hypopharynx 6910. Among other uses, the accelerometer 6920 may comprise one example implementation of sensing element 110 (FIG. 1A). In some examples, the accelerometer 6920 may enable sensing respiration information, among other physiologic information (e.g., body position, activity, etc.) at least because at least some portions of the hypopharynx exhibit motion/behavior during respiration and which is indicative of phasic respiratory information.

However, in some examples, the accelerometer (XL) 6920 may be delivered to a desired target tissue (e.g., hypopharynx 6910) via incisions, pathways (e.g., intravascular), etc. independent of (e.g., without) using the implant-access incision 6810.

In some examples, the accelerometer 6920 may comprise at least some of substantially the same features and/or attributes as: U.S. Pat. No. 11,324,950 issued on May 10, 2022, titled ACCELEROMETER-BASED SENSING FOR SLEEP DISORDERED BREATHING (SDB) CARE, filed Oct. 19, 2018 under Ser. No. 16/092,384; U.S. 2023-0119173, published on Apr. 20, 2023, titled RESPIRATION DETECTION, and filed Sep. 2, 2020 under Ser. No. 16/977,664; U.S. 2023-0095780 published on Mar. 30, 2023, titled SLEEP DETECTION FOR SLEEP DISORDERED BREATHING (SDB) CARE, and filed Sep. 4, 2020 under Ser. No. 16/978,470; and WO 2022-261311 published on Dec. 15, 2022, titled RESPIRATION SENSING, and filed Jun. 9, 2022 under Serial Number PCT/US2022/032821, each of which is herein incorporated by reference.

FIGS. 18, 19, 20, and 21 are diagrams schematically representing example devices for sensing and applying stimulation. The devices of FIGS. 18-21 may include an implementation of, and/or include, at least some of substantially the same features of any device, engine, and/or control portion of FIGS. 1A-2B and 6-10C, and/or be used to implement the timing diagrams and/or methods of any of FIGS. 3A-3C, 5A-5C, 17A-17J, and/or sense and/or stimulate any target tissue illustrated by FIGS. 11-16. In some examples, FIGS. 18-21 may be implemented independent of FIGS. 3A-5C. For example, each of the devices include electrode arrangements, which may be used to implement and/or include sensing elements and/or stimulation elements. In some examples, each electrode arrangement may be used to provide only sensing or only stimulation. In some examples, each electrode arrangement may be used to provide both sensing and stimulation. For example, sub-sets of electrodes of the arrangement may be used to provide sensing and other sub-sets used to provide stimulation. In some examples, respective electrodes of the electrode arrangement may provide sensing and stimulation at different times. In some examples, respective electrodes of the electrode arrangements may be used to provide sensing or stimulation and other electrodes of the electrode arrangements may be used to both provide sensing and stimulation. In some examples, each electrode arrangement may comprise an example implementation of, and/or at least some of substantially the same features and attributes as sensing elements and stimulation elements (and related arrangements or circuits) described in association with various examples described in association with at least FIGS. 1A-1C, 2A-2B, and 6-9. The common elements and features are not repeated for ease of reference.

More specifically, FIG. 18 is a diagram including a front view schematically representing deployment 1200 of an example IMD 1222 including electrode arrangements 1210R, 1210L, 1213R, 1213L, 1214R, 1214L, 1216R, 1216L deployed for sensing from and/or stimulating a plurality of target tissues. In some examples, the target tissues include hypoglossal nerves 1260R, 1260L, IHM-innervating nerves 1290R, 1290L, iSL nerves 1240R, 1240L, and/or phrenic nerves 1295R, 1295L. In some examples, the target tissues may additionally and/or alternatively include muscles innervated by or elicited as part of reflux response driven by such nerves, including but not limited to genioglossus muscle, IHMs, diaphragm muscles, such as those illustrated at least in connection with FIGS. 11-16.

The IMD 1222 comprises an IPG 1233 and the electrode arrangements 1210R, 1210L, 1213R, 1213L, 1214R, 1214L, 1216R, 1216L. As shown in FIG. 18, in some examples, the IPG 1233 (which may include sensing circuit 152 and/or stimulation circuit 154 of FIG. 1B) may be chronically implanted in a pectoral region 1202 of the patient 1215 and the electrode arrangements 1210R, 1210L, 1213R, 1213L, 1214R, 1214L, 1216R, 1216L may be chronically implanted in a head-and-neck region 1205 of the patient. In some examples, the IPG 1233 in combination with the electrode arrangements 1210R, 1210L, 1213R, 1213L, 1214R, 1214L, 1216R, 1216L may form the sensor element 110 and stimulation element 120 of FIG. 1A, in some examples, and may sense the respiratory information from and stimulate target tissues.

Among other features, it will be understood that, in some examples, a body of a lead supports the electrode arrangement, while extending between the IPG 1233 and one or more of the electrode arrangements 1210R, 1210L, 1213R, 1213L, 1214R, 1214L, 1216R, 1216L, such as leads illustrated in connection with at least FIGS. 6-7B.

Moreover, in some examples, the IPG 1233 may be formed on a smaller scale and/or different shape to be amenable for implantation in the head-and-neck region 1205 instead of pectoral region 1202. Accordingly, in some such examples, the IPG 1233 may comprise, or may be sometimes be referred to as, a microstimulator. In some of these examples, the sensor 110 (e.g., a sensing element) and/or stimulation element 120 may be wholly incorporated into and/or on the IPG 1233, while in some examples, a portion of the sensing element and/or stimulation element 120 may be separate from the IPG 1233 and connected to the IPG 1233 via a lead (wired) or via a wireless connection.

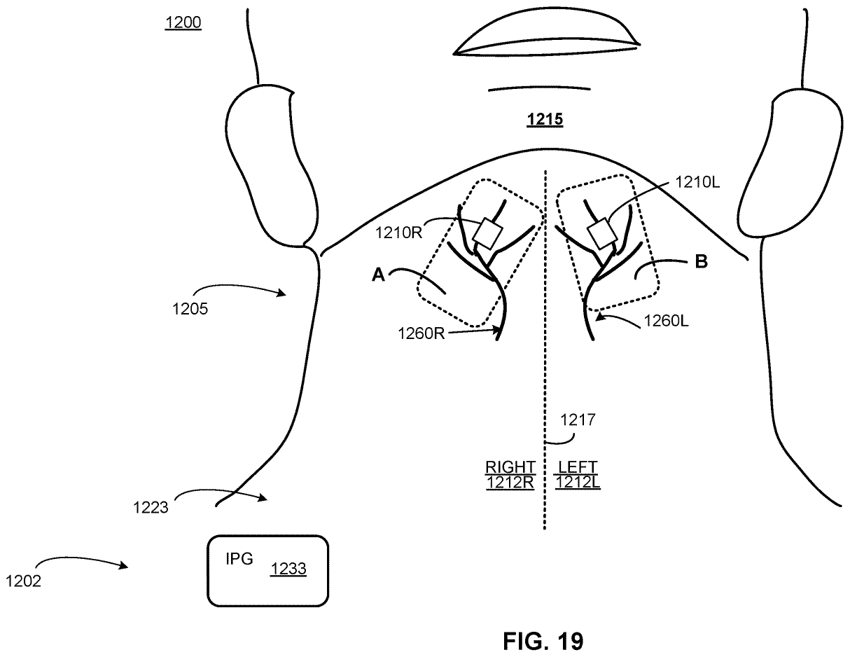

In some examples, each of the respective electrode arrangements 1210R, 1210L, 1213R, 1213L, 1214R, 1214L, 1216R, 1216L may be implanted within each of the respective locations A, B, C, D, E, F, G, H of the patient 1215 which are located respectively on right and left sides 1212R, 1212L in the head-and-neck 1205 region of the patient 1215, as shown with respect to the sagittal midline 1217. Different combinations of the target nerves 1240R, 1240L, 1260R, 1260L, 1290R, 1290L, 1295R, 1295L may be used to sense respiration information (and/or other physiologic information) and/or provide stimulation thereto, such as described previously in connection with FIG. 17C. In some examples, any of the target nerves 1240R, 1240L, 1260R, 1260L, 1290R, 1290L, 1295R, 1295L, or combinations thereof, may be used to sense a first respiration parameter using the respective electrode arrangements 1210R, 1210L, 1213R, 1213L, 1214R, 1214L, 1216R, 1216L. The effect of stimulating each specific target nerve 1240R, 1240L, 1260R, 1260L, 1290R, 1290L, 1295R, 1295L is previously described above, at least in connection with FIGS. 11-16 and example devices are further illustrated by at least FIGS. 19-21. It will be understood that the particular locations of the electrode arrangements 1210R, 1210L, 1213R, 1213L, 1214R, 1214L, 1216R, 1216L (e.g., at least one electrode) provide just one example and that such locations are also representative of many different target tissues and locations at which the respective electrode arrangement may be located consistent with accessibility of the respective nerves, muscles, other tissues, etc.

In some examples, different target nerves or other tissue may be stimulated depending on the sensed respiratory information. In some examples, multiple tissues may stimulated at the same time or different times depending on the type of obstruction. While stimulation of just the hypoglossal nerve 1260R, 1260L (or some branches thereof) may be effective in increasing upper airway patency to a sufficient degree to ameliorate obstructive sleep apnea in a large majority of appropriate patients when using certain types of implantable neurostimulation devices, some patients may benefit from stimulation of an IHM-innervating nerve 1290L and/or 1290R, the iSL nerve 1240R and/or 1240L, and/or the phrenic nerve 1295R and/or 1295L in addition to, or instead of, stimulation of the hypoglossal nerve 1260L and/or 1260R. Moreover, for a single patient, obstructive sleep apnea arising from certain positions of the head-and-neck and/or of their body (e.g., supine, lateral decubitis, etc.) and/or of their body-mass index (BMI) may be treated more effectively by stimulating an IHM-innervating nerve (e.g., 1290L, 1290R), and stimulating or not stimulating the hypoglossal nerve (e.g., 1260R and/or 1260L). In some such examples, upon detecting that a patient is in a certain body position (e.g., supine), stimulation of the IHM-innervating nerve (e.g., 1290R, 1290L) may be implemented. In some examples the stimulation may implemented using at some of substantially the same features and attributes as described in Verzal, et al., WO 2022/246320, published on Nov. 11, 2022, entitled “MULTIPLE TARGET STIMULATION THERAPY FOR SLEEP DISORDERED BREATHING”, corresponding to U.S. National Stage Application, Serial No. ______, filed on ______, and published on ______ as U.S. Publication ______, which is incorporated herein by reference in it entireties for its teachings.

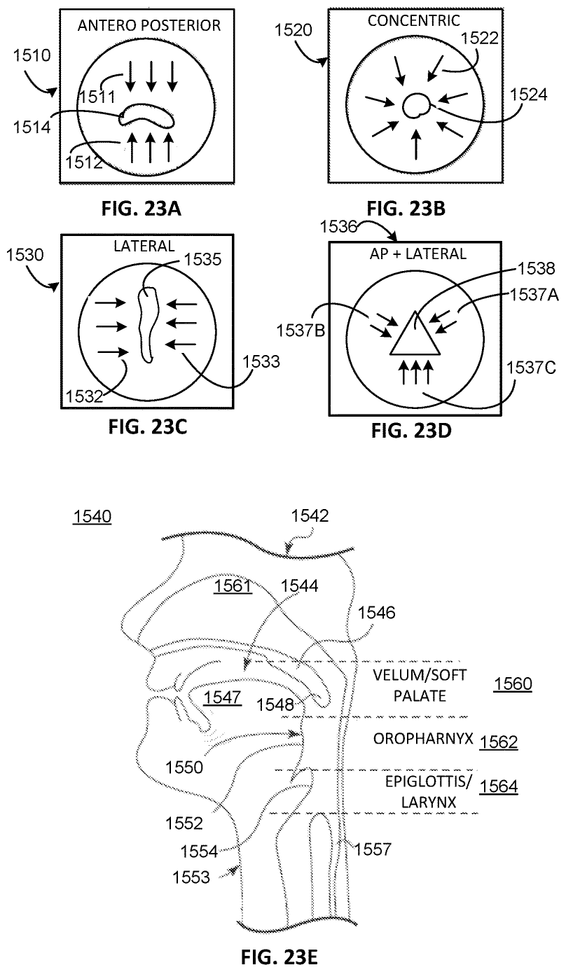

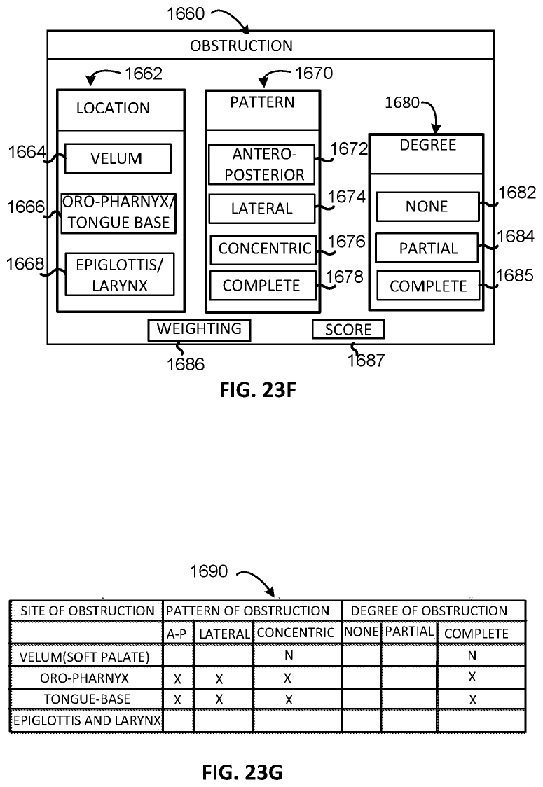

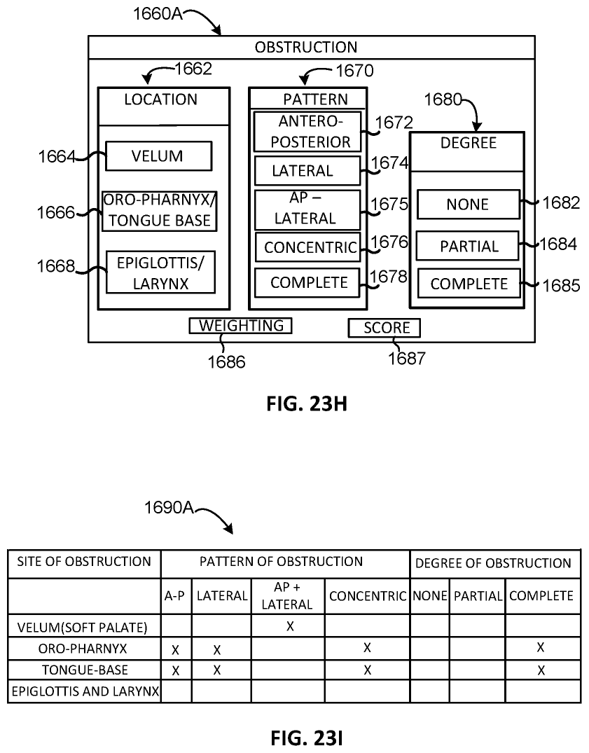

In addition, because each of the target nerves (e.g., iSL nerve 1240R, 1240L, the hypoglossal nerve 1260R, 1260L, IHM-innervating nerve 1290L, 1290R) innervates and/or elicits several different muscle groups which may influence upper airway patency, stimulation may be applied at several different locations (e.g., different nerve portions) of the branches of the particular target nerve in order to specifically stimulate and/or elicit those respective different muscle groups (e.g., sometimes without stimulating muscle groups which may produce an antagonistic action or unrelated action). Such stimulation at the respective different locations may occur simultaneously, sequentially, alternately, etc., depending on which nerves (or muscles) are being stimulated, depending on when the stimulation occurs relative to the respective respiratory phases (or portions of each phase) of a respiratory period of the patient's breathing, and/or based on other factors. Moreover, stimulation may be alternated, sequenced, etc., between portions of a single nerve (e.g., hypoglossal) and/or may be alternated, sequenced, etc. among multiple different nerves including the iSL nerve 1240R, 1240L, the hypoglossal nerve 1260R, 1260L, the IHM-innervating nerve 1290R, 1290L, and/or the phrenic nerve 1295R, 1295L, among other nerves identified as target tissues 5130 in association in FIG. 17C or elsewhere throughout the present disclosure. It will be understood that the muscles innervated by such nerves also may comprise stimulation targets. At least some of these examples are further described herein in association with at least FIGS. 23A-23I.

FIG. 19 is a diagram including a front view schematically representing deployment 1200 of an example IMD 1223 including at least one electrode arrangement deployed for sensing and/or stimulating a hypoglossal nerve. The IMD 1223 may comprises an implementation of, and/or at least some of substantially the same features and attributes as, the example IMD 1222 in FIG. 18, except the electrode arrangements are deployed proximate to the hypoglossal nerves 1260R, 1260L on the left and right sides 1212R, 1212L.

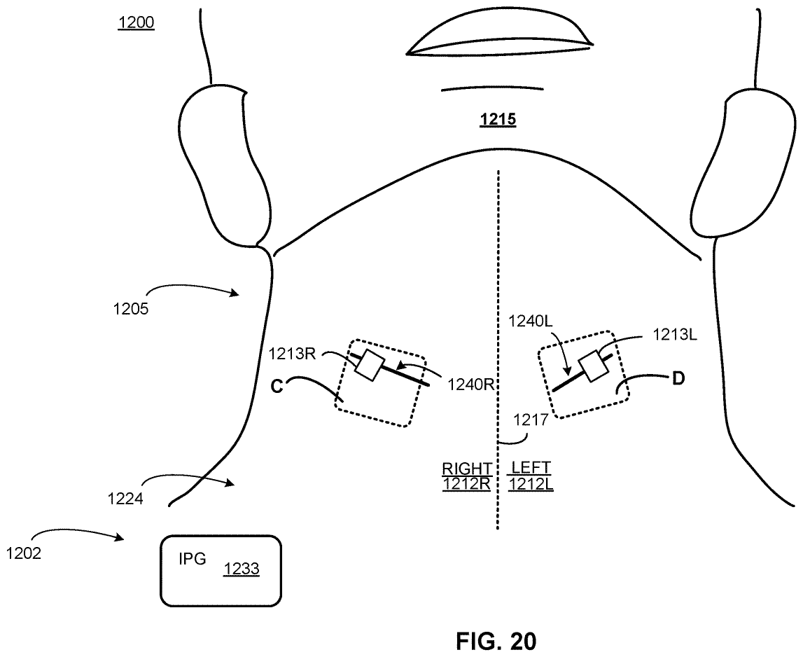

FIG. 20 is a diagram including a front view schematically representing deployment 1200 of an example IMD 1224 including at least one electrode arrangement deployed for sensing and/or stimulating an iSL nerve. The IMD 1224 may comprises an implementation of, and/or at least some of substantially the same features and attributes as, the example IMD 1222 in FIG. 18, except the electrode arrangements are deployed proximate to the iSL nerves 1240R, 1240L on the left and right sides 1212R, 1212L.

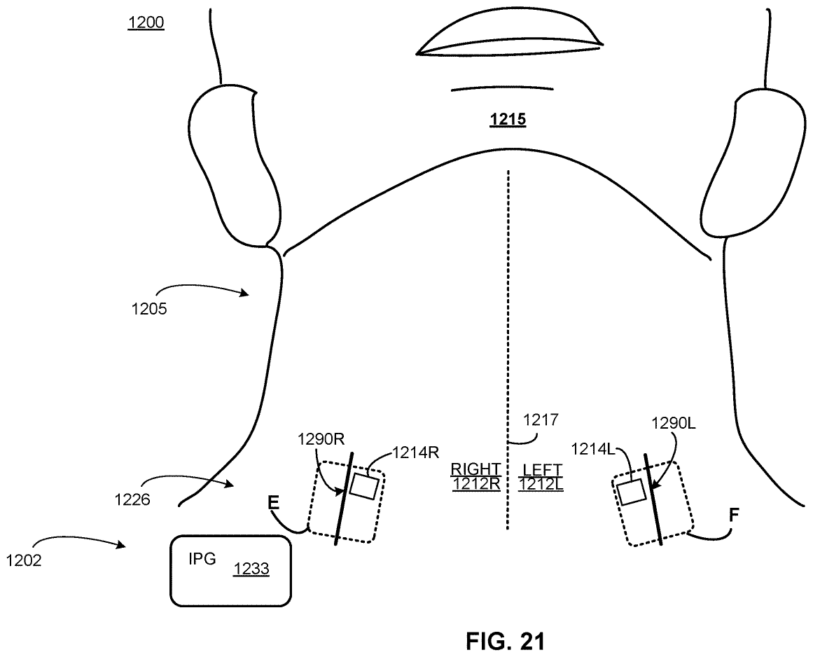

FIG. 21 is a diagram including a front view schematically representing deployment 1200 of an example IMD 1226 including at least one electrode arrangement deployed for sensing and/or stimulating an IHM-innervating nerve. The IMD 1226 may comprises an implementation of, and/or at least some of substantially the same features and attributes as, the example IMD 1222 in FIG. 18, except the electrode arrangements are deployed proximate to the IHM-innervating nerves 1290R, 1290L on the left and right sides 1212R, 1212L.

Each of FIGS. 18-21 illustrate example devices, e.g., IMDs, with stimulation electrode arrangements which are bilaterally disposed on both the right and left sides 1212R, 1212L of the head-and-neck region 1205 of the patient 1215. Examples are not so limited, and at least one of the electrode arrangements may be disposed one side and not the other (e.g., on the left side 1212L or on the right side 1212R) and/or may be disposed on both sides, but used to sense and/or stimulate on one side. In some examples, an electrode arrangement disposed on a first side proximate to a first target tissue (e.g., left side 1212L) may be used to sense respiratory information and an electrode arrangement disposed on the second side proximate to the first target tissue (e.g., right side 1212R) may be used to stimulate the first target tissue. In some examples, an electrode arrangement disposed on a first side proximate to a first target tissue (e.g., left side 1212L) may be used to sense respiratory information and an electrode arrangement disposed on the second side proximate to a second target tissue (e.g., right side 1212R) may be used to stimulate the second target tissue, which is different from the first target tissue.

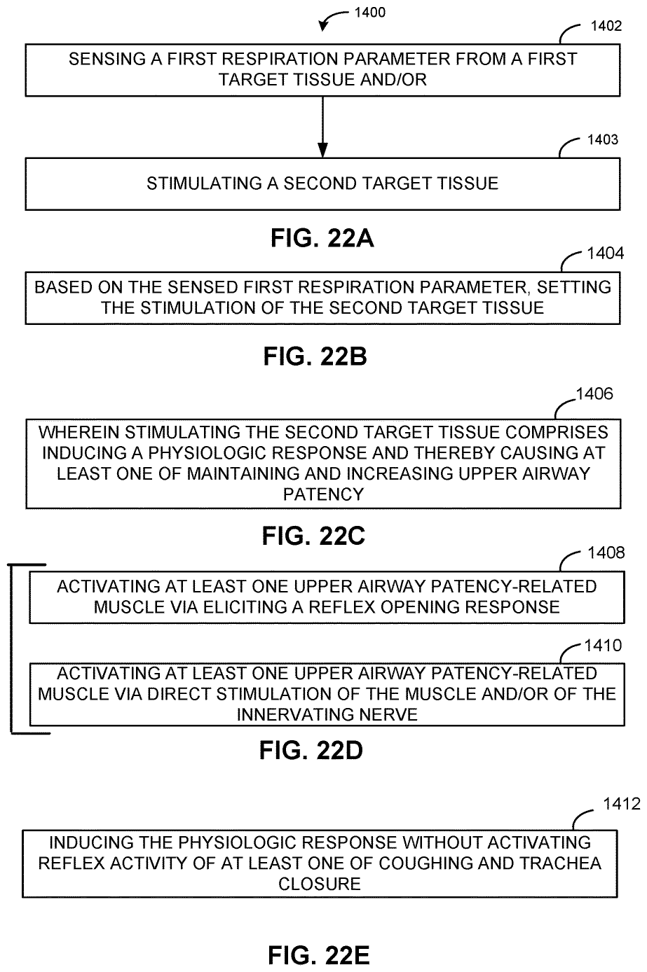

FIGS. 22A-22E are flow diagrams illustrating example methods for sensing and/or applying stimulation. The methods illustrated by FIGS. 22A-22E may be implemented by any device, engine, and/or control portion of FIGS. 1A-2B, 6-10C, and 19-21 and/or be used to implement the timing diagrams and/or methods of any of FIGS. 3A-3C, 5A-5C, 17A-17B, and/or sense and/or stimulate any target tissue illustrated by FIGS. 11-16.

In some examples, as shown at 1402 of FIG. 22A, a method 1400 may comprise sensing a first respiration parameter from a first target tissue, and/or, at 1403, stimulating a second target tissue. In some examples, as shown at 1404 of FIG. 22B, the method 1400 may further comprise setting the stimulation of the second target tissue based on the sensed first respiration parameter. As previously described, setting the stimulation may be used to control the timing of stimulation, the amplitude of the stimulation, and/or selection of the second target tissue, among other settings, and which may be applied in real time or at other times.

In some examples, as shown at 1406 of FIG. 22C, simulating the second target tissue comprises inducing a physiologic response and thereby causing maintaining and/or increasing upper airway patency. For example, as shown at 1408 and 1410 of FIG. 22D, the physiologic response may comprise activating at least one upper airway patency-related muscle via eliciting a reflex opening response (e.g., elicited via CNS). In some examples, the physiologic response may comprise activating an upper airway patency-related muscle via stimulation of the efferent nerve fibers of the target nerve and/or stimulating the muscle directly. In some examples, at least some upper airway patency-related muscles (innervated by upper airway patency-related motor nerves) include a genioglossus muscle, an IHM, and/or other muscles. As shown at 1412 of FIG. 22E, the method 1400 may comprise inducing the physiologic response without activating reflex activity of coughing and/or trachea closure.

Example methods may include and/or be directed to any of the variations as described herein, and are not limited to that illustrated by FIGS. 22A-22E.

FIGS. 23A-23D are diagrams including front and side views schematically representing patient anatomy and example methods relating to collapse patterns associated with upper airway patency. More specifically, FIGS. 23A-23D are a series of diagrams schematically representing at least some different upper airway collapse patterns, including an anterior-posterior (AP) collapse pattern (FIG. 23A), a concentric collapse pattern (FIG. 23B), a lateral collapse pattern (FIG. 23C), and an anterior-posterior (AP)—lateral collapse pattern (FIG. 23D). In addition to observing such collapse patterns and/or other collapse patterns, at least some aspects of such collapse patterns may be measured, such as via impedance sensing using implanted electrodes (e.g., sensing elements and/or stimulation elements), using externally applied arrays of electrodes, etc. such as described and illustrated in association with at least FIGS. 23A-23D. By determining an upper airway collapse pattern, some example arrangements may determine whether to apply stimulation via a hypoglossal nerve, via an iSL nerve, via afferent branches of a glossopharyngeal nerve, via an IHM-innervating nerve (including which single or multiple portions thereof to stimulate), via other non-hypoglossal nerve related to upper airway patency (e.g., glossopharyngeal nerve), and/or combinations of these nerves including unilateral and bilateral options.

At least some more specific details regarding FIGS. 23A-23D are further described below in relation to at least FIGS. 23E-23I.

FIGS. 23E-23I are block diagrams schematically representing example devices and/or example methods relating to collapse patterns associated with upper airway patency.

FIG. 23F is a block diagram schematically representing an example sorting tool 1660 by which to sort and weigh a location, pattern, and degree of obstruction or patency. As shown in FIG. 23F, obstruction sorting tool 1660 includes functions for location detection 1662, pattern detection 1670, and degree detection 1680. In general terms, the location detection function 1662 operates to identify a site along the upper airway at which an obstruction occurs and which is believed to cause sleep disordered breathing. In one example, the location detection function 1662 includes a velum (soft palate) parameter 1664, an oropharynx-tongue base parameter 1666, and an epiglottis/larynx parameter 1668. Each respective parameter denotes an obstruction identified in the respective physiologic territories of the velum (soft palate), oropharnyx-tongue base, and epiglottis which are generally illustrated for an example patient in FIG. 23E. In one aspect, these distinct physiologic territories define an array of vertical strata within the upper airway. Moreover, each separate physiologic territory (e.g., vertical portion along the upper airway) exhibits a distinct characteristic behavior regarding obstructions and associated impact on breathing during sleep. Accordingly, each physiologic territory responds differently to implantable upper airway stimulation.

With this in mind, the velum (soft palate parameter 1664 denotes obstructions taking place in the level of the region of the velum (soft palate), as illustrated in association with FIG. 23F. FIG. 23E is a diagram including a side view schematically representing at least some anatomical features of the upper airway, as well as different sites or levels at which obstruction may occur. By determining a site or location of upper airway collapse, some example arrangements may determine whether to apply stimulation via a hypoglossal nerve, via an IHM-innervating nerve (including which portions thereof to stimulate), via a iSL nerve, via other non-hypoglossal nerve related to upper airway patency, and/or combinations of these nerves including unilateral and bilateral options, such as but not limited to the glossopharyngeal nerve.

As shown in FIG. 23E, a diagram 1540 provides a side sectional view (cross hatching omitted for illustrative clarity) of a head-and-neck region 1542 of a patient. In particular, an upper airway portion 1550 extends from the mouth region 1544 to a neck portion 1553. The upper airway portion 1550 includes a velum (soft palate) region 1560, an oropharynx region 1562, and an epiglottis region 1564. The velum (soft palate) region 1560 includes an area extending below sinus 1561, and including the soft palate 1560, approximately to the point at which tip 1548 of the soft palate 1546 meets a portion of tongue 1547 at the back of the mouth region 1544. The oropharynx region 1562 extends approximately from the tip of the soft palate 1546 (when in a closed position) along the base 1552 of the tongue 1547 until reaching approximately the tip region of the epiglottis 1554. The epiglottis-larynx region 1562 extends approximately from the tip of the epiglottis 1554 downwardly to a point above the esophagus 1557.

As will be understood from FIG. 23E, each of these respective regions 1560, 1562, 1564 within the upper airway correspond the respective velum parameter 1664, oropharynx parameter 1666, and epiglottis parameter 1668, respectively of FIG. 23F.

With further reference to FIG. 23F, in general terms the pattern detection function 1670 enables detecting and determining a particular pattern of an obstruction of the upper airway. In one example, the pattern detection function 1670 includes an antero-posterior parameter 1672, a lateral parameter 1674, a concentric parameter 1676, and composite parameter 1678.

The antero-posterior parameter 1672 of pattern detection function 1670 (FIG. 23F) denotes a collapse of the upper airway that occurs in the antero-posterior orientation, as further illustrated in the diagram 1510 of FIG. 23A. In FIG. 23A, arrows 1511 and 1512 indicate one example direction in which the tissue of the upper airway collapses, resulting in the narrowed air passage 1514. FIG. 23A is also illustrative of a collapse of the upper airway in the soft palate region 1560, whether or not the collapse occurs in an antero-posterior orientation. For example, in some instances, the velum (soft palate) region 1560 exhibits a concentric (e.g., circular) pattern of collapse, as shown in diagram 1520 of FIG. 23B.

The concentric parameter 1676 of pattern detection function 1670 (FIG. 23F) denotes a collapse of the upper airway that occurs in a concentric orientation, as further illustrated in the diagram 1520 of FIG. 23B. In FIG. 23B, arrows 1522 indicate the direction in which the tissue of the upper airway collapses, resulting in the narrowed air passage 1524.

The lateral parameter 1674 of pattern detection function 1670 (FIG. 23F) denotes a collapse of the upper airway that occurs in a lateral orientation, as further illustrated in the diagram 1530 of FIG. 23C. In FIG. 23C, arrows 1532 and 1533 indicate the direction in which the tissue of the upper airway collapses, resulting in the narrowed air passage 1535.

The composite parameter 1678 of pattern detection function 1670 (FIG. 23F) denotes a collapse of the upper airway portion that occurs via a combination of the other mechanisms (lateral, concentric, antero-posterior) or that is otherwise ill-defined from a geometric viewpoint but that results in a functional obstruction of the upper airway portion.

With further reference to obstruction sorting tool 1660 of FIG. 23F, in general terms the degree detection function or module 1680 indicates a relative degree of collapse or obstruction of the upper airway portion. In some examples, the degree detection function 1680 includes a none parameter 1682 a partial collapse parameter 1684, and a complete collapse parameter 1685. In some examples, the none parameter 1682 may correspond to a collapse of 25 percent or less, while the partial collapse parameter 1684 may correspond to a collapse of between about 25 to 75%, and the complete collapse parameter 1685 may correspond to a collapse of greater than 75 percent. In some examples, the at least one respiration parameter sensed from the first target tissue may include respiratory obstruction information, such as neural activity which is indicative of a relative degree of collapse or obstruction of the upper airway.

It will be understood that various patterns of collapse occur at different levels of the upper airway portion and that the level of the upper airway in which a particular pattern of collapse appears can vary from patient-to-patient.

In some examples, obstruction sorting tool 1660 comprises a weighting function 1686 and score function 1687. In general terms, the weighting function 1686 assigns a weight to each of the location, pattern, and/or degree parameters (FIG. 23F) as one or more those respective parameters can contribute more heavily to the patient exhibiting sleep disordered breathing or to being more responsive to implantable upper airway stimulation. More particularly, each respective parameter (e.g., antero-posterior 1672, lateral 1674, concentric 1676, composite 1678) of each respective detection modules (e.g., pattern detection function 1670) is assigned a weight corresponding to whether or not the patient is eligible for receiving implantable upper airway stimulation. Accordingly, the presence of or lack of a particular pattern of obstruction (or location or degree) will be become part of an overall score (according to score parameter 1687) for an obstruction vector indicative how likely the patient will respond to therapy via an implantable upper airway stimulation system.

FIG. 23G is diagram (e.g., chart) 1690 schematically representing an index or scoring tool to sort and weigh a location, pattern, and degree of obstruction or patency for a particular patient. Chart 1690 combines information regarding location (1662 in FIG. 23F), pattern (1670 in FIG. 23F), and degree (1680 in FIG. 23F) into a single informational grid or tool by which the obstruction is documented for a particular patient and by which appropriate stimulation settings may be determined and applied according to the various examples of the present disclosure, such as but not limited to those in association with at least FIGS. 1-22E, etc.

FIGS. 23H-23I are diagrams 1660A, 1690A like the diagrams 1660, 1690 of FIGS. 23F-23G, respectively, except with FIGS. 23H-23I further addressing an anterior-posterior (AP) lateral collapse pattern, which is depicted in diagram 1536 of FIG. 23D, provided as a parameter 1675 of a pattern detection function 1670 of FIG. 23H, and incorporated into the index of FIG. 23I.

As shown in FIG. 23D, this pattern comprises a combination of the anterior-posterior pattern (FIG. 23A) and the lateral pattern (FIG. 23C) with arrows 1537A, 1537B, 1537C indicating example directions in which the tissue of the upper airway collapses, resulting in the narrowed air passage 1538. The narrowed air passage 1538 may comprise a triangular shape in some examples. In some examples, the AP-lateral collapse pattern at a velum/soft palate (1560 in FIGS. 23E, 1664 in FIGS. 23H-23) may respond better (e.g., increase patency) to stimulation of an infrahyoid-based patency tissue than a concentric collapse pattern having a similar severity/completeness as the AP-lateral collapse pattern at the soft palate.

Accordingly, in some examples, the information sensed and collected via at least FIGS. 23F-23I may be used to determine whether to apply stimulation via a hypoglossal nerve, via a iSL nerve, via an IHM-innervating nerve (including which single portion or multiple portions thereof to stimulate), via other non-hypoglossal nerves related to upper airway patency, and/or combinations of these nerves including unilateral and bilateral options.

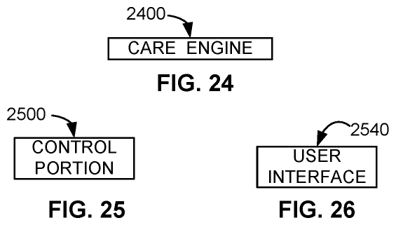

FIG. 24 schematically represents an example care engine 2400 by which at least some of substantially the same features and attributes of the examples of FIGS. 11-23I may be implemented in association with control portion 2500 (FIG. 25). In some examples, care engine 2400 may comprise at least some of substantially the same features and/or attributes as care engine 800 of FIG. 9.

FIG. 25 schematically represents an example control portion 2500 by which at least some of substantially the same features and attributes of the examples of FIGS. 11-23I may be implemented in association with control portion care engine 2400 (FIG. 24). In some examples, care engine 2400 may comprise at least some of substantially the same features and/or attributes as care engine 800 of FIG. 9.

FIG. 26 schematically represents an example user interface 2540 by which at least some of substantially the same features and attributes of the examples of FIGS. 11-23I may be implemented in association with control portion 2500 (FIG. 25) and/or care engine 2400 (FIG. 24). In some examples, user interface 2540 may comprise at least some of substantially the same features and/or attributes as user interface 940 of FIG. 10C.

Although specific examples have been illustrated and described herein, a variety of alternate and/or equivalent implementations may be substituted for the specific examples shown and described without departing from the scope of the present disclosure. This application is intended to cover any adaptations or variations of the specific examples discussed herein.
Example A1. A method comprising sensing a first respiration parameter from a first target tissue and/or stimulating a second target tissue.
Example A2. The method of example A1, wherein the first respiration parameter comprises respiratory phase information and/or respiratory obstruction information.
Example A3. The method of example A2, wherein the respiratory phase information comprises inspiratory phase.
Example A4. The method of example A1, comprising each of sensing the first respiration parameter from the first target tissue and stimulating the second target tissue.
Example A5. The method of example A4, wherein sensing of the first respiration parameter is timed independent of the stimulating the second target tissue.
Example A6. The method of example A1, wherein the first target tissue comprises a first portion of a first respiratory-related tissue and the second target tissue comprises a second portion of the first respiratory-related tissue.
Example A7. The method of example A6, wherein the first respiratory-related tissue comprises an upper airway patency-related motor nerve.
Example A8A. The method of example A7, wherein the nerve is selected from the group consisting of: a hypoglossal nerve; an infrahyoid-muscle (IHM)-innervating nerve; and a combination thereof.
Example A8B. The method of example A6, wherein the first respiratory-related tissue comprises an upper airway reflex-related sensory nerve selected from the group consisting of: an internal superior laryngeal nerve, an afferent branch of a glossopharyngeal nerve; and a combination thereof.
Example A8C. The method of example A6, wherein the respiratory-related tissue comprises a phrenic nerve and/or a diaphragm muscle.
Example A9. The method of example A6, wherein sensing the first respiration parameter from the first target tissue comprises bilaterally sensing the first respiration parameter from the first target tissue on a first lateral side and a second lateral side of a patient, and/or stimulating the second target tissue comprises bilaterally stimulating the second target tissue on the first lateral side and the second lateral side of the patient.
Example A10. The method of example A1, wherein the first target tissue comprises a first respiratory-related tissue and the second target comprises a second respiratory-related tissue different from the first tissue.
Example A11. The method of example A10, wherein the first respiratory-related tissue comprises a first upper airway patency-related motor nerve and the second respiratory-related tissue comprises a second upper airway patency-related motor nerve different from first upper airway patency-related motor nerve.
Example A12A. The method of example A11, wherein the first nerve and the second nerve comprises nerves selected from the group consisting of: a hypoglossal nerve; an infrahyoid-muscle (IHM)-innervating nerve; and a combination thereof.
Example A12B. The method of example A10, wherein the first respiratory-related tissue and the second respiratory-related tissue comprise upper airway reflex-related sensory nerves selected from the group consisting of: an internal superior laryngeal nerve; afferent branch of a glossopharyngeal nerve; and a combination thereof.
Example A12C. The method of example A10, wherein the first respiratory-related tissue and/or the second respiratory-related tissue comprise a phrenic nerve.
Example A13. The method of example A10, wherein the first target tissue and second target tissue comprise at least two of the group consisting of: the hypoglossal nerve; the internal superior laryngeal nerve; the IHM-innervating nerve; afferent branch of a glossopharyngeal nerve; and the phrenic nerve.
Example A14. The method of example A10, wherein the first target tissue and the second target tissue are selected from the hypoglossal nerve and IHM-innervating nerve.
Example A15. The method of example A10, wherein the first target tissue and the second target tissue are selected from the hypoglossal nerve, the internal superior laryngeal nerve, and the IHM-innervating nerve.
Example A16. The method of example A10, wherein sensing the first respiration parameter from the first target tissue comprises bilaterally sensing the first respiration parameter from the first target tissue on a first lateral side and a second lateral side of a patient, and/or stimulating the second target tissue comprises bilaterally stimulating the second target tissue on the first lateral side and the second lateral side of the patient
Example A17. The method of example A10, wherein the first respiratory-related tissue comprises a first muscle and the second respiratory-related tissue comprises a first nerve.
Example A18. The method of example A10, wherein the first respiratory-related tissue comprises a first nerve and the second respiratory-related tissue comprises a second nerve.
Example A19. The method of example A10, wherein the first respiratory-related tissues comprises a first nerve and the second respiratory-related tissue comprises a first muscle and, optionally, a second nerve.
Example A20. The method of example A10, wherein the first respiratory-related tissue comprises a first muscle and the second respiratory-related tissue comprises a second muscle.
Example A21. The method of example A10, wherein the first respiratory-related tissue comprises a first upper airway patency-related motor nerve and the second respiratory-related tissue comprises a second upper airway patency-related motor nerve different from first upper airway patency-related motor nerve.
Example A22A. The method of example A21, wherein the first upper airway patency-related motor nerve and/or the second upper airway patency-related motor nerve comprise a nerve selected from the group consisting of: a hypoglossal nerve; an infrahyoid-muscle (IHM)-innervating nerve; and a combination thereof.
Example A22B. The method of example A10, wherein the first respiratory-related tissue comprises an upper airway reflex-related sensory nerve selected from the group consisting of: an internal superior laryngeal nerve, an afferent branch of a glossopharyngeal nerve; and a combination thereof.
Example A22C. The method of example A10, wherein the respiratory-related tissue comprises a phrenic nerve and/or a diaphragm muscle.
Example A23. The method of example A1, wherein the sensing of the first respiratory parameter is performed via: electromyography (EMG), and/or electroneurography (ENG).
Example A24. The method of example A1, wherein the sensing of the first respiratory parameter includes sensing biopotential from mixed tissue source.
Example A25. The method of example A10, wherein stimulating the second target tissue comprises treating sleep disordered breathing by promoting upper airway patency.
Example A26. The method of example A25, wherein the sleep disordered breathing comprises obstructive sleep apnea.
Example A27. The method of example A1, further comprising, based on the sensed first respiration parameter, setting the stimulation of the second target tissue.
Example A28. The method of example A27, wherein setting the stimulation comprises: setting timing of the stimulation according to the first respiration parameter; setting an amplitude of the stimulation according to the first respiration parameter; and/or selecting the second target tissue (from a set of targets) based on the first respiration parameter.
Example A29. The method of example A1, wherein the first respiration parameter comprises respiratory phase information including inspiration and/or expiration.
Example A30. The method of example A1, comprising sensing the first respiration parameter by sensing neural activity and, using the sensed neural activity, determining the first respiration parameter.
Example A31. The method of example A30, wherein the neural activity is associated with mechanoreceptors that are affected by respiration.
Example A32. The method of example A30, further comprising sensing a second respiration parameter using the sensed neural activity and/or additionally sensed neural activity, the second respiration parameter comprising respiratory obstruction information.
Example A33. The method of example A32, wherein the respiratory obstruction information is indicative of a degree of upper airway obstruction.
Example A34. The method of example A32, further comprising stimulating the second target tissue based on the first respiration parameter and the second respiration parameter by: setting a timing of the stimulation according to the first respiration parameter; and setting an amplitude of the stimulation according to the second respiration parameter.
Example A35. The method of example A1, wherein the first target tissue and/or the second target tissue comprise an internal superior laryngeal nerve.
Example A36. The method of example A35, wherein the first target tissue and the second target tissue comprise the internal superior laryngeal nerve.
Example A37. The method of example A35, wherein the first target tissue comprises the internal superior laryngeal nerve and the second target tissue comprises a different portion of the internal superior laryngeal nerve than the first target tissue.
Example A38. The method of example A35, wherein stimulating the second target tissue comprises selectively stimulating an afferent nerve fiber of the internal superior laryngeal nerve.
Example A39. The method of example A35, wherein sensing the first respiratory parameter from the internal superior laryngeal nerve comprises sensing neural activity of mechanoreceptors that are affected by respiration.
Example A40. The method of example A35, wherein stimulating the internal superior laryngeal nerve elicits a reflex opening of the upper airway.
Example A41A. The method of example A40, wherein the elicited reflex opening recruits a plurality of upper airway patency-related muscles for promoting upper airway patency
Example A41B. The method of example A35, further comprising stimulating the second target tissue based on the first respiration parameter by: setting a timing of the stimulation according to the first respiration parameter; setting an amplitude of the stimulation according to the first respiration parameter; and/or selecting the second target tissue (from a set of targets) based on the first respiration parameter.
Example A42. The method of example A1, wherein the first target tissue and/or the second target tissue comprises an infrahyoid-muscle (IHM)-innervating nerve and/or an IHM.
Example A43. The method of example A42, wherein the first target tissue and the second target tissue comprise different portions of the IHM-innervating nerve.
Example A44. The method of example A42, wherein the first target tissue comprises the IHM-innervating nerve and/the IHM, and the second target tissue comprises: the IHM-innervating nerve; the IHM; and/or a hypoglossal nerve (e.g., distal portion of the HGN).
Example A45. The method of example A42, wherein sensing the first respiratory parameter from the IHM-innervating nerve and/or the IHM comprises sensing neural activity (from the IHM-innervating nerve or IHM) that is phasic with respiration.
Example A46. The method of example A45, wherein the neural activity has an onset that precedes the onset of inspiration and remains through an inspiratory phase of a respiratory cycle.
Example A47. The method of example A46, wherein the neural activity increases in amplitude and/or duty cycle in response to an upper airway obstruction.
Example A48. The method of example A42, wherein the stimulating the second target tissue activates an upper airway patency-related muscle.
Example A49. The method of example A42, wherein stimulating the second target tissue comprising causing displacement of the thyroid cartilage inferiorly, and thereby causing stiffening of a pharyngeal wall of the patient which occurs remotely therefrom.
Example A50. The method of example A42, further comprising stimulating the second target tissue based on the first respiration parameter by: setting a timing of the stimulation according to the first respiration parameter; setting an amplitude of the stimulation according to the first respiration parameter; and/or selecting the second target tissue (from a set of targets) based on the first respiration parameter.
Example A51. The method of example A1, wherein the first target tissue and/or the second target tissue comprise a hypoglossal nerve and/or a genioglossus muscle.
Example A52. The method of example A51, wherein the first target tissue and the second target tissue comprise different portions of the hypoglossal nerve.
Example A53. The method of example A51, wherein sensing the first respiratory parameter from the hypoglossal nerve comprises sensing neural activity that is phasic with respiration
Example A54. The method of example A53, wherein the neural activity has an onset that precedes the onset of inspiration and remains through an inspiratory phase of a respiratory cycle.
Example A55. The method of example A51, wherein the neural activity increases in amplitude and/or duty cycle in response to an upper airway obstruction.
Example A56. The method of example A51, wherein the stimulating the second target tissue activates an upper airway patency-related muscle (e.g., genioglossus muscle).
Example A57. The method of example A51, wherein stimulating the second target tissue causes the tongue muscle to stiffen and to protrude by activating a genioglossus muscle, and thereby promoting upper airway patency (e.g., dilating the upper airway).
Example A58. The method of example A51, further comprising stimulating the second target tissue based on the first respiration parameter by: setting a timing of the stimulation according to the first respiration parameter; setting an amplitude of the stimulation according to the first respiration parameter; and/or selecting the second target tissue (from a set of targets) based on the first respiration parameter.
Example A59. The method of example A1, wherein stimulating the second target tissue comprises inducing a physiologic response and thereby causing maintaining and/or increasing upper airway patency.
Example A60. The method of example A59, wherein the physiologic response causes recruiting an upper airway patency-related muscle, and/or activating an upper airway patency-related muscle.
Example A61. The method of example A60, wherein the upper airway patency-related muscle includes at least one muscle selected from the group consisting of: a genioglossus muscle and an IHM.
Example A62. The method of example A60, further comprising inducing the physiologic response without activating reflex activity of coughing and/or trachea closure.
Example A63. The method of example A1, further comprising selecting the second target tissue from a set of target tissues based on the first respiratory parameter, wherein the first respiratory parameter includes respiratory obstruction information.
Example A64. The method of example A63, wherein the set of target tissues comprise a set of nerves and muscles innervated and/or elicited by the set of nerves.
Example A65. The method of example A64, wherein the set of nerves comprise: a hypoglossal nerve; an internal superior laryngeal nerve; an infrahyoid-muscle (IHM)-innervating nerve; a glossopharyngeal nerve; and a phrenic nerve.
Example B1. A device comprising a sensing and/or stimulation element to sense a first respiration parameter from a first target tissue, and/or stimulate a second target tissue.
Example B2A. The device of example B1, wherein the device comprises the sensing element and the stimulation element.
Example B2B. The device of example B1, wherein the sensing and/or stimulation element comprise an electrode arrangement including sensing and stimulations elements.
Example B3. The device of example B1, wherein the device further comprises: a sensing circuit to receive sensed physiologic information from the sensing and/or stimulation element, as sensed from the first target tissue; and/or a stimulation circuit to deliver a stimulation signal to the sensing and/or stimulation element for application to the second target tissue.
Example B4. The device of example B3, wherein the device comprises the sensing circuity and the stimulation circuit, and the sensing element forms part of a sensor.
Example B5. The device of example B3, wherein the device further comprises an event detector to detect the first respiration parameter from the sensed physiological information and, in response, to output a signal to the stimulation circuit to set stimulation of the second target tissue.
Example B6. The device of example B5, wherein the output signal sets the stimulation including: setting a timing of the stimulation according to the first respiration parameter; setting an amplitude of the stimulation according to the first respiration parameter; and/or selecting the second target tissue (from a set of targets) based on the first respiration parameter.
Example B7. The device of example B1, wherein the first respiration parameter comprises respiratory phase information and/or respiratory obstruction information, wherein the respiratory phase information optionally comprises inspiratory phase.
Example B8. The device of example B1, wherein sensing of the first respiration parameter is timed independent of the stimulating the second target tissue.
Example B9. The device of example B1, wherein the first target tissue comprises a first portion of a first respiratory-related tissue and the second target tissue comprises a second portion of the first respiratory-related tissue.
Example B10. The device of example B9, wherein the first-respiratory related tissue comprises an upper airway patency-related motor nerve.
Example B11A. The device of example B10, wherein the nerve is selected from the group consisting of: a hypoglossal nerve; an internal superior laryngeal nerve; and a combination thereof.
Example B11B. The device of example B9, wherein the first respiratory-related tissue comprises an upper airway reflex-related sensory nerve selected from the group consisting of: an internal superior laryngeal nerve, an afferent branch of a glossopharyngeal nerve; and a combination thereof.
Example B11C. The device of example B9, wherein the respiratory-related tissue comprises a phrenic nerve and/or a diaphragm muscle.
Example B12. The device of example B1, wherein sensing the first respiration parameter from the first target tissue comprises bilaterally sensing the first respiration parameter from the first target tissue on a first lateral side and a second lateral side of a patient, and/or stimulating the second target tissue comprises bilaterally stimulating the second target tissue on the first lateral side and the second lateral side of the patient.
Example B13. The device of example B1, wherein the first target tissue comprises a first respiratory-related tissue and the second target comprises a second respiratory-related tissue different from the first tissue.
Example B14. The device of example B13, wherein the first respiratory-related tissue comprises a first upper airway patency-related motor nerve and the second respiratory-related comprises a second upper airway patency-related nerve different from first upper airway patency-related motor nerve.
Example B15A. The device of example B14, wherein the first nerve and the second nerve comprises nerves selected from the group consisting of: a hypoglossal nerve; an internal superior laryngeal nerve; and a combination thereof.
Example B15B. The device of example B13, wherein the first respiratory-related tissue and the second respiratory-related tissue comprise upper airway reflex-related nerves selected from the group consisting of: an internal superior laryngeal nerve; afferent branch of a glossopharyngeal nerve; and a combination thereof.
Example B15C. The device of example B13, wherein the first respiratory-related tissue and/or the second respiratory-related tissue comprise a phrenic nerve.
Example B16. The device of example B13, wherein the first target tissue and second target tissue comprise at least two of the group consisting of: the hypoglossal nerve; the internal superior laryngeal nerve; the IHM-innervating nerve; an afferent branch of the glossopharyngeal nerve; and the phrenic nerve.
Example B17. The device of example B13, wherein the first target tissue and the second target tissue are selected from the hypoglossal nerve and IHM-innervating nerve.
Example B18. The device of example B13, wherein the first target tissue and the second target tissue are selected from the hypoglossal nerve, the internal superior laryngeal nerve, and the IHM-innervating nerve.
Example B19. The device of example B13, wherein the first respiratory-related tissue comprises a first muscle and the second respiratory-related tissue comprises a first nerve.
Example B20. The device of example B13, wherein the first respiratory-related tissue comprises a first nerve and the second respiratory-related tissue comprises a second nerve.
Example B22. The device of example B13, wherein the first respiratory-related tissue comprises a first nerve and the second respiratory-related tissue comprises a first muscle and, optionally, a second nerve.
Example B23. The device of example B13, wherein the first respiratory-related tissue comprises a first muscle and the second respiratory-related tissue comprises a second muscle.
Example B24. The device of example B13, wherein the first respiratory-related tissue comprises a first upper airway patency-related motor nerve and the second respiratory-related tissue comprises a second upper airway patency-related motor nerve different from first upper airway patency-related motor nerve.
Example B25A. The device of example B24, wherein the first upper airway patency-related motor nerve and/or the second upper airway patency-related motor nerve comprise a nerve selected from the group consisting of: a hypoglossal nerve; an infrahyoid-muscle (IHM)-innervating nerve; and a combination thereof.
Example B25B. The device of example B13, wherein the first respiratory-related tissue comprises an upper airway reflex-related sensory nerve selected from the group consisting of: an internal superior laryngeal nerve, an afferent branch of a glossopharyngeal nerve; and a combination thereof.
Example B25C. The device of example B12, wherein the respiratory-related tissue comprises a phrenic nerve and/or a diaphragm muscle.
Example B26. The device of example B1, wherein stimulating the second target tissue comprises treating sleep disordered breathing by promoting upper airway patency, wherein the sleep disordered breathing optionally comprises obstructive sleep apnea.
Example B27. The device of example B1, wherein the first respiration parameter comprises respiratory phase information including inspiration and/or expiration.
Example B28. The device of example B1, wherein the sensing and/or stimulation element is to sense the first respiration parameter by sensing neural activity and, using the sensed neural activity, determining the first respiration parameter.
Example B29. The device of example B28, wherein the neural activity is associated with mechanoreceptors that are affected by respiration.
Example B30. The device of example B29, wherein the sensing and/or stimulation element is to sense a second respiration parameter using the sensed neural activity and/or additionally sensed neural activity, the second respiration parameter comprising respiratory obstruction information.
Example B31A. The device of example B30, wherein the respiratory obstruction information is indicative of a degree of upper airway obstruction.
Example B31B. The device of example B31A, wherein the sensing and/or stimulation element is to stimulate the second target tissue based on the first respiration parameter and the second respiration parameter by: a timing of the stimulation set according to the first respiration parameter; and/or an amplitude of the stimulation set according to the second respiration parameter.
Example B32. The device of example B1, wherein the first target tissue and/or the second target tissue comprise an internal superior laryngeal nerve.
Example B33. The device of example B32, wherein the first target tissue and the second target tissue comprise the internal superior laryngeal nerve.
Example B34. The device of example B32, wherein the first target tissue comprises the internal superior laryngeal nerve and the second target tissue comprises a different portion of the internal superior laryngeal nerve than the first target tissue.
Example B35. The device of example B32, wherein the sensing and/or stimulation element is to stimulate the second target tissue comprises selectively stimulating an afferent nerve fiber of the internal superior laryngeal nerve.
Example B36. The device of example B32, wherein sensing the first respiratory parameter from the internal superior laryngeal nerve comprises sensing neural activity of mechanoreceptors that are affected by respiration.
Example B38. The device of example B32, wherein stimulating the internal superior laryngeal nerve elicits a reflex opening of the upper airway.
Example B39. The device of example B38, wherein the elicited reflex opening recruits a plurality of upper airway patency-related muscles for promoting upper airway patency.
Example B40. The device of example B32, wherein the sensing and/or stimulation element is to stimulate the second target tissue based on the first respiration parameter by: setting a timing of the stimulation according to the first respiration parameter; setting an amplitude of the stimulation according to the first respiration parameter; and/or selecting the second target tissue (from a set of targets) based on the first respiration parameter.
Example B41. The device of example B1, wherein the first target tissue and/or the second target tissue comprises an infrahyoid-muscle (IHM)-innervating nerve and/or an IHM.
Example B42. The device of example B41, wherein the first target tissue and the second target tissue comprise different portions of the IHM-innervating nerve.
Example B43. The device of example B41, wherein the first target tissue comprises the IHM-innervating nerve and/the IHM, and the second target tissue comprises: the IHM-innervating nerve; the IHM; and/or a hypoglossal nerve (e.g., distal portion of the HGN).
Example B44. The device of example B41, wherein the sensing and/or stimulation element is to sense the first respiratory parameter from the IHM-innervating nerve and/or the IHM by sensing neural activity (from the IHM-innervating nerve or IHM) that is phasic with respiration.
Example B45. The device of example B44, wherein the neural activity has an onset that precedes the onset of inspiration and remains through an inspiratory phase of a respiratory cycle.
Example B46. The device of example B45, wherein the neural activity increases in amplitude and/or duty cycle in response to an upper airway obstruction.
Example B47. The device of example B41, wherein the sensing and/or stimulation element is to stimulate the second target tissue to activate an upper airway patency-related muscle (e.g., IHM if stim AC loop or genioglossus muscle is stim HGN).
Example B48. The device of example B41, wherein the sensing and/or stimulation element is to stimulate the second target tissue, and thereby cause displacement of the thyroid cartilage inferiorly, and stiffening of a pharyngeal wall of the patient which occurs remotely therefrom.
Example B49. The device of example B41, wherein the sensing and/or stimulation element are to stimulate the second target tissue based on the first respiration parameter by: setting a timing of the stimulation according to the first respiration parameter; setting an amplitude of the stimulation according to the first respiration parameter; and/or selecting the second target tissue (from a set of targets) based on the first respiration parameter.
Example B50. The device of example B1, wherein the first target tissue and/or the second target tissue comprise a hypoglossal nerve and/or a genioglossus muscle.
Example B51. The device of example B50, wherein the first target tissue and the second target tissue comprise different portions of the hypoglossal nerve.
Example B52. The device of example B50, wherein the sensing and/or stimulation element are to sense the first respiratory parameter from the hypoglossal nerve by sensing neural activity that is phasic with respiration.
Example B53. The device of example B52, wherein the neural activity has an onset that precedes the onset of inspiration and remains through an inspiratory phase of a respiratory cycle.
Example B54. The device of example B50, wherein the neural activity increases in amplitude and/or duty cycle in response to an upper airway obstruction.
Example B55. The device of example B50, wherein the sensing and/or stimulation element is to stimulate the second target tissue to activate an upper airway patency-related muscle (e.g., genioglossus muscle).
Example B56. The device of example B50, wherein stimulating the second target tissue causes the tongue muscle to stiffen and to protrude by activating a genioglossus muscle, and thereby promoting upper airway patency (e.g., dilating the upper airway).
Example B57. The device of example B50, wherein the sensing and/or stimulation element is to stimulate the second target tissue based on the first respiration parameter by: setting a timing of the stimulation according to the first respiration parameter; setting an amplitude of the stimulation according to the first respiration parameter; and/or selecting the second target tissue (from a set of targets) based on the first respiration parameter.
Example B58. The device of example B1, wherein the sensing and/or stimulation element is to stimulate the second target tissue to induce a physiologic response and thereby causing maintaining and/or increasing upper airway patency.
Example B59. The device of example B58, wherein the physiologic response causes: recruiting an upper airway patency-related muscle; and/or activating an upper airway patency-related muscle.
Example B60. The device of example B58, wherein the upper airway patency-related muscle includes at least one muscle selected from the group consisting of: a genioglossus muscle (e.g., protrusion muscles) and an IHM.
Example B61. The device of example B58, wherein the stimulation induces the physiologic response without activating reflex activity of coughing and/or trachea closure.
Example B62. The device of example B1, further comprising circuitry to select the second target tissue from a set of target tissues based on the first respiratory parameter, wherein the first respiratory parameter includes respiratory obstruction information.
Example B63. The device of example B62, wherein the set of target tissues comprise a set of nerves and muscles innervated and/or elicited by the set of nerves.
Example B64. The device of example B63, wherein the set of nerves comprise: a hypoglossal nerve; an internal superior laryngeal nerve; an infrahyoid-muscle (IHM)-innervating nerve; an afferent branch of a glossopharyngeal nerve; and a phrenic nerve.

1. A device comprising:
a clock to generate a clock signal;
a sensing circuit to periodically sense a signal based on the clock signal; and
a stimulation circuit to output a stimulation pulse train relative to the periodic sensing of the signal based on the clock signal.

2. The device of claim 1, wherein the stimulation pulse train comprises a plurality of stimulation pulses, and
wherein an interval between the periodic sensing of the signal and a stimulation pulse of the stimulation pulse train is constant.

3. The device of claim 1, wherein the sensing circuit is to periodically sense the signal every first predetermined number of cycles of the clock signal.

4. The device of claim 1, wherein the stimulation pulse train comprises a plurality of stimulation pulses, and
wherein the stimulation circuit is to output each stimulation pulse of the plurality of stimulation pulses every second predetermined number of cycles of the clock signal.

5. A device comprising:
a clock to generate a clock signal;
a sensing circuit to sense a signal beginning every first predetermined number of cycles of the clock signal;
an event detector to generate a start signal in response to detecting an event; and
a stimulation circuit to output a stimulation pulse train, the stimulation pulse train comprising a plurality of stimulation pulses, each stimulation pulse beginning every second predetermined number of cycles of the clock signal,
wherein the stimulation circuit begins a first stimulation pulse of the stimulation pulse train a third predetermined number of cycles of the clock signal after the beginning of a previous sensing of the signal in response to the start signal.

6. The device of claim 5, wherein the first predetermined number equals the second predetermined number.

7. The device of claim 5, wherein the first predetermined number is an integer multiple or an integer divisor of the second predetermined number.

8. The device of claim 5, wherein the sensing circuit comprises a first counter to count cycles of the clock signal and begin to sense the signal and reset the first counter in response to the count of the first counter equaling the first predetermined number of cycles of the clock signal.

9. The device of claim 8, wherein the stimulation circuit comprises a second counter to count cycles of the clock signal and begin a stimulation pulse and reset the second counter in response to the count of the second counter equaling the second predetermined number of cycles of the clock signal.

10. The device of claim 9, wherein the count of the first counter is offset with respect to the count of the second counter by the third predetermined number of cycles.

11. The device of claim 5, wherein the sensing circuit continues to sense the signal between stimulation pulse trains.

12. An implantable medical device comprising:
a clock to generate a clock signal;
a sensing circuit to sense a physiologic signal of a patient beginning every first predetermined number of cycles of the clock signal;
an event detector to generate a start signal in response to detecting a physiologic event of the patient; and
a stimulation circuit to output a stimulation pulse train to a nerve of a patient, the stimulation pulse train comprising a plurality of stimulation pulses, each stimulation pulse beginning every second predetermined number of cycles of the clock signal,
wherein the stimulation circuit begins a first stimulation pulse of the stimulation pulse train a third predetermined number of cycles of the clock signal after the beginning of a previous sensing of the physiologic signal in response to the start signal.

13. The implantable medical device of claim 12, wherein the sensing circuit senses a physiologic signal of the patient beginning a fourth predetermined number of cycles after the beginning of a previous stimulation pulse of the stimulation pulse train.

14. The implantable medical device of claim 13, wherein the third predetermined number of cycles is less than the fourth predetermined number of cycles.

15. The implantable medical device of claim 12, wherein the physiologic event of the patient comprises inspiration or expiration.

16. The implantable medical device of claim 12, wherein the physiologic signal comprises a cardiac signal, a muscle signal, or a nerve signal.

17. The implantable medical device of claim 12, wherein each stimulation pulse of the stimulation pulse train comprises a cathodic portion and an anodic portion.

18. The implantable medical device of claim 12, wherein the nerve innervates the tongue and soft palate of the patient.

19. The implantable medical device of claim 12, wherein the implantable medical device is configured to treat sleep disordered breathing.

20-24. (canceled)

25. The device of claim 1, wherein the sensing circuit comprises a first counter to count cycles of the clock signal and begin to sense the signal and reset the first counter in response to the count of the first counter equaling a first predetermined number of cycles of the clock signal, and
wherein the stimulation circuit comprises a second counter to count cycles of the clock signal and begin a stimulation pulse and reset the second counter in response to the count of the second counter equaling a second predetermined number of cycles of the clock signal.
